(19) United States
(12) Patent Application Publication
(10) Pub. No.: US 2025/0346932 A1
(43) Pub. Date: Nov. 13, 2025

(54) IMMOBILIZED ENZYMES FOR THE BIOELECTRIC PRODUCTION OF FORMATE AND FORMIC ACID

(71) Applicant: Anodyne Chemistries, Inc., Burnaby (CA)

(72) Inventors: Emmanuelle DAVIES, Burnaby (CA); Robert GREENE, Burnaby (CA); Phillip DANBY, Burnaby (CA); Antony D. ST-JACQUES, Burnaby (CA)

(21) Appl. No.: 19/275,193

(22) Filed: Jul. 21, 2025

Related U.S. Application Data
(63) Continuation of application No. PCT/CA2025/050646, filed on May 1, 2025.
(60) Provisional application No. 63/641,038, filed on May 1, 2024.

(51) Int. Cl. C12P 7/62; C12N 9/02; C12N 11/14; C12P 7/42

(52) U.S. Cl. CPC C12P 7/62; C12N 9/0008; C12N 11/14; C12P 7/42; C12Y 102/01002

(57) ABSTRACT

The present invention relates to enzymatic reactor cells and related methods of use, e.g., to produce formic acid and/or formate by using an enzymatic reactor cell, wherein the enzymatic reactor cell includes a surface, a linker, and one or more enzymes.

### CROSS-REFERENCE TO RELATED APPLICATIONS

This application is a Continuation Application of International Application No. PCT/CA2025/050646, filed May 1, 2025, which claims the benefit of and priority to U.S. Provisional Application No. 63/641,038, filed May 1, 2024, which is incorporated herein by reference in its entirety.

### REFERENCE TO AN ELECTRONIC SEQUENCE LISTING

The contents of the sequence electronic listing (ANOD_002_01US_SeqList_ST26.xml; Size: 207,336 bytes; and Date of Creation: Jul. 18, 2025) are herein incorporated by reference in its entirety.

### BACKGROUND OF THE INVENTION

Enzymatic bioelectrocatalysis is increasing in popularity for the production of various commodities such as methanol, or other forms of energy. Oxidoreductase enzymes are key enzymes in microorganisms and have been utilized or mutated to catalyze crucial redox reactions. Cofactors and/or electrodes can also be utilized to enable electron transfer from the active site of the enzyme and the substrate. The overall reaction pathway to create a single commodity, however, can be complex, as it can require multiple enzymes, cofactors and substrates, adding to the complexity of enzymatic bioelectrocatalysis to create a final product or commodity. Use of multiple such oxidoreductase enzymes and cofactors or other enzymes has been used for example in bioreactors or fermenters with limited success, as it has been difficult to manipulate and control the intended reactions. The use of such enzymatic bioelectrocatalysis to provide efficient and intended production of commodities needs improvement. Such ideas include the use of a reactor cell or multiple reactor cells wherein an enzyme such as formate dehydrogenase is immobilized to a surface, and the reaction is supported by an electrode for controlled and efficient electron transfer in the reaction. Improvements in this field are still needed.

### SUMMARY OF THE INVENTION

The disclosure provides enzymatic reactor cells and related methods of use, e.g., to produce a compound or product, such as formate or formic acid, by using an enzymatic reactor cell. In a specific embodiment, the enzymatic reactor cell, includes a surface, a surface linker or electrode surface linker, and one or more enzymes, wherein the surface is directly linked to the surface or electrode surface linker and the surface linker is further directly linked to the enzyme. In a specific embodiment, the surface linker may be an electrode surface linker. In a specific embodiment, the enzymatic reactor cell has the following formula I:

surface—electrode surface linker—enzyme (Formula I).

In a specific embodiment, Formula I can specifically be as follows:

[surface—electrode surface linker—enzyme]ₙ₍₁₋₁₀₎ (Formula I);
wherein when n is 2-10, each of the 2-10 [surface—electrode surface linker—enzyme] comprises a different surface, electrode surface linker and/or enzyme from the other [surface—electrode surface linker—enzyme].

In a specific embodiment, Formula II can specifically be as follows:

[surface—surface linker—enzyme]ₙ₍₁₋₁₀₎ (Formula II);
wherein when n is 2-10, each of the 2-10 [surface—surface linker—enzyme] comprises a different surface, surface linker and/or enzyme from the other [surface—electrode surface linker—enzyme].

In another specific embodiment, n of Formula I or Formula II is 1, 2, 3, 4, 5, 6, 7, 8, 9, or 10. In another specific embodiment, n of Formula I or Formula II is 11, 12, 13, 14, 15, 16, 17, 18, 19, or 20.

In another embodiment, the surface is conductive, thereby allowing electron transfer between the enzyme and the surface or electrode surface. In another specific embodiment the electron transfer travels from the enzyme to the surface. In another specific embodiment the electron transfer travels from the surface to the enzyme. In a specific embodiment, the enzyme is an oxidoreductase enzyme.

In a specific embodiment, the electrode surface linker is conductive, thereby allowing electron transfer from the oxidoreductase enzyme and the electrode surface and comprises a surface binding moiety (SBM), and in a specific embodiment the electrode surface linker providing election transfer comprises a peptide, protein, a chemical polymer, or polynucleotide described herein.

In a specific embodiment, the oxidoreductase enzyme comprises formate dehydrogenase. Thus, in one embodiment, the enzymatic reactor cell produces formate or formic acid or a combination thereof. In a specific embodiment, the enzyme reactor cell comprises formate dehydrogenase. In another embodiment, the enzymatic reactor cell produces formate or formic acid. In embodiments, the formate ion can be produced in the presence of appropriate counter ions and isolated as the corresponding salt—e.g., sodium to make sodium formate, potassium to make potassium formate, etc. In another embodiment, the enzymatic reactor cells, fusion proteins, linkers, or methods may be from PCT application PCT/US2023/078253, filed Oct. 30, 2023, which is incorporated by reference in its entirety herein.

### BRIEF DESCRIPTION OF THE DRAWINGS

Further features and advantages of the present disclosure will become apparent from the following detailed description, taken in combination with the appended drawings, in which:

FIG. 1 illustrates by mass spectrometry the successful conjugation of graphite MBP (SEQ ID NO: 25) to a glutaraldehyde nanowire.

FIG. 2 illustrates current density of conjugated formate dehydrogenase absorbed to the glassy carbon disk surface in comparison to just enzyme or just the glassy carbon disk.

FIG. 3 shows the concentration of formate detected after 2 hour chronoamperometry reactions for enzyme-linker (n=3) and no enzyme (glassy carbon electrode alone) (n=3), and just enzyme with no linker (n=2).

FIG. 4 illustrates a reactor cell for producing formate from bicarbonate.

FIG. 5 illustrates a reactor cell for producing formate from CO₂.

FIG. 6 illustrates a reactor cell for producing formic acid from bicarbonate.

FIG. 7 illustrates a reactor cell for producing formic acid from CO₂.

FIG. 8 shows a bar chart of the average current density (over 2 hours of chronoamperometry) of conjugated formate dehydrogenase (enzyme-linker) immobilized on carbon black and its comparison to pure carbon black (carbon black blank).

FIG. 9 shows the concentration of formate detected after 60 minutes of chronoamperometry of enzyme-linker absorbed carbon black and carbon black blank.

FIG. 10 presents the faradaic efficiency of the immobilized mutated enzyme on graffoil. The results are compared to those obtained from control experiments using graffoil alone and the wildtype enzyme alone.

FIG. 11 shows the concentration of formate detected from the mutated-enzyme, enzyme only, and graffoil.

FIG. 12 provides a schematic depiction of the flow electrolyzer used in the study.

FIG. 13A and FIG. 13B provide voltage profiles for the enzyme-linker under constant-current electrolysis at 4 mA/cm². (FIG. 13A) Cell voltage profile. (FIG. 13B) Cathode potential profile. Note that a current ramp-up procedure was adopted before the current density reached 4 mA/cm². The electrolysis duration at 4 mA/cm² was 1 h.

FIG. 14 provides a graph of formate production for the enzyme-linker and the bare graphite felt after constant current electrolysis at 4 mA/cm². The electrolysis duration was 1 h. Error bar represents standard deviations from 3 individual measurements.

FIG. 15A and FIG. 15B provide schematic depictions of the flow electrolyzer with different cathode configurations. (FIG. 15A) Electrolyzer supplied with CO₂(g). (FIG. 15B) Electrolyzer supplied with CO₂(aq), in which the CO₂-saturated catholyte was fed into the catholyte chamber.

FIG. 16 shows a bar chart of formate production, after 1 hour of constant electrolysis, for bare carbon paper, enzyme only, and the enzyme-linker. Error bars represent standard deviations from individual measurements (n≥2).

FIG. 17 provides a graph of formate production over time using bare carbon paper, enzyme only, and enzyme-linker. Error bars represent standard deviations from individual measurements (n≥2).

FIG. 18 shows a bar chart of formate production, after 1 hour of constant electrolysis, n on bare carbon paper, enzyme only, and enzyme e-linker).

### DETAILED DESCRIPTION

The following terms are defined below.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors. As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “a linker sequence” is intended to mean a sequence that bridges the surface binding entity, e.g., inorganic surface entity, with the organic binding entity, such as an enzyme. As used herein, a linker sequence may comprise one or both of an active linker and/or a passive linker. Thus, a linker sequence may, for example, comprise the amino acid sequence of protein G from Streptococcus or streptavidin from Streptomyce, or may be a simple amino acid sequence or simply a single bond, such as a covalent bond. The linkage can also be a non-covalent bond to the enzyme and/or surface. Organic binding entities include both synthetic carbon-based compounds as well as biologically-derived molecules. The linker may include additional functional features, such as being a cofactor for an enzyme, including the enzyme directly linked to the linker.

The term “surface binding motif” or SBM is intended to mean a molecule with specific and selective affinity for an organic or inorganic substance, such as, e.g., gold, silica, silicon, silver, plastic, polystyrene, cellulose (e.g., nitrocellulose), PDMS, chlorine doped polypyrrole/polypropylene, zinc oxide, iron oxide, titanium oxide, graphite, carbon paper, carbon felt, SWCNT, carbon black, and graphene. An SBM may be a peptide or polypeptide.

The term “covalent fusion” is intended to mean the joining of two or more genes that encode separate peptides or proteins. The terms “polypeptide”, “protein” and “peptide” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” or “peptide” means one or more chains of amino acids, wherein each chain comprises amino acids covalently linked by peptide bonds, and wherein said polypeptide or protein or peptide can comprise a plurality of chains non-covalently and/or covalently linked together by peptide bonds, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. Thus, a “polypeptide” or a “protein” can comprise one (termed “a monomer”) or a plurality (termed “a multimer”) of amino acid chains.

The term “fusion protein” means a protein comprised of at least two different amino acid sequences and generated within an organism such as E. coli. An inorganic surface binding peptide expressed with an A or G protein or a linker is an example of a fusion protein.

As used herein, the “alignment” of two or more protein/amino acid sequences may be performed using the alignment program ClustalW2, available at www.ebi.ac.uk/Tools/msa/clustalw2/. The following default parameters may be used for Pairwise alignment: Protein Weight Matrix=Gonnet; Gap Open=10, Gap Extension=0.1. Any sequence alignment or determination of sequence identity of proteins or amino acid sequences is determined by the software or alignment program described herein.

The term “specifically binds” means that a molecule reacts or associates more frequently, more rapidly, with greater duration and/or with greater affinity with a particular target molecule, e.g., a pathogen or surface, than it does with alternative molecules, e.g., pathogens or other surfaces. It is also understood by reading this definition that, a molecule that specifically or preferentially binds to a first target may or may not specifically or preferentially bind to a second target. As such, “specific binding” does not necessarily require (although it can include) exclusive binding.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

In this disclosure, the word “comprising” is used in a non-limiting sense to mean that items following the word are included, but items not specifically mentioned are not excluded.

It will be understood that in embodiments which comprise or may comprise a specified feature or variable or parameter, alternative embodiments may consist, or consist essentially of such features, or variables or parameters. A reference to an element by the indefinite article “a” does not exclude the possibility that more than one of the elements is present, unless the context clearly requires that there be one and only one of the elements.

In this disclosure the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5, etc). In this disclosure the singular forms an “an”, and “the” include plural referents unless the content clearly dictates otherwise. Thus, for example, reference to a composition containing “a compound” includes a mixture of two or more compounds.

In this disclosure term “or” is generally employed in its sense including “and/or” unless the content clearly dictates otherwise.

### Embodiments of the Invention

Bioelectrocatalysis includes reactions that are catalyzed by biologically active materials in association with electrically conductive electrodes.

Enzymatic bioelectrocatalysis is a specific form of bioelectrocatalysis or electrocatalysis using an enzyme for catalyzing a certain reaction. In one general example of enzymatic bioelectrocatalysis, enzymes are associated with an electrode, including electrode linkers, in a manner that allows electron transfer between the electrode and the enzymes. Such electron transfer allows the continued function of each enzyme over many catalyzed reactions, including a series of reactions to obtain a final desired compound or product. The term “enzyme(s)” as used herein thus relates to a biologically based catalytic mechanism, and can comprise a protein that is both wild-type or mutated for any intended reaction by the user. Nonlimiting examples of other biologically based catalytic materials can include eukaryotic cells, prokaryotic cells, cellular organelles, nucleic acid enzymes (i.e. deoxyribozymes), and the like.

Oxidoreductase enzymes are specific biocatalytic proteins that can catalyse the coupled oxidation and reduction with a substrate and/or cofactor, thus, transferring an electron(s) with the involvement of an electrode linker and/or a cofactor of the enzyme. In a specific embodiment of the present invention, the oxidoreductase enzyme can be used in a single reaction or in a series of reactions with other enzymes or oxidoreductase enzymes wherein the oxidoreductase enzyme is directly linked to, for example, an electrode linker. In a specific embodiment, the enzymes or oxidoreductase enzyme is directly linked to an electrode linker in a device, biodevice or reactor cell that contains the reaction or series of reactions if more than one enzyme or oxidoreductase enzyme is used.

In a specific embodiment, the device, biodevice or reactor cell allows for the immobilization of an enzyme or oxidoreductase enzymes. In a specific embodiment, the device, biodevice or reactor cell comprises a surface that allows for immobilization of the enzyme or oxidoreductase enzyme. In another embodiment, the surface may be an electrode surface. This surface may include conductive or non-conductive material.

In another embodiment, the surface may have active groups or other means for attaching an enzyme or other means for linking the enzyme to the surface. In one specific embodiment, a linker is specifically used to link to the surface (such as covalently or non-covalently) and then link to the enzyme. In a specific embodiment, the linker is covalently linked to the surface and covalently linked to the enzyme. In another embodiment, the linker can be covalently linked to just the surface or the enzyme. In another embodiment, the linker may be linked to the surface or enzyme by non-covalent means.

In one embodiment, the enzymatic process can utilise more than one device, biodevice or reactor cell. In one embodiment, several enzymatic reactor cells are constructed in series or in parallel. In another embodiment, several enzymatic reactor cells are constructed in series or in parallel each allowing for a different catalytic reaction. In a specific embodiment, a series of reactor cells can be used to comprise more than one enzymatic reactions to carry out an enzymatic pathway, i.e., there is more than one enzyme used to achieve an end product or compound from a starting compound.

In one embodiment, the enzymatic bioelectrocatalysis is performed in an enzymatic reactor cell.

In a specific embodiment, the enzymatic reactor cell, comprises a surface, such as an electrode surface, or non-electrode surface; a linker or electrode surface linker; and one or more enzymes, wherein the electrode surface is linked to the electrode surface linker and the surface linker is further directly linked to the oxidoreductase enzyme. In a specific embodiment, the enzyme is a mutant or wildtype form of formate dehydrogenase. In a specific embodiment, the enzyme is a formate dehydrogenase, or a formate dehydrogenase in specific conditions to produce formate or formic acid.

Formate dehydrogenase is an enzyme that is traditionally known to catalyze the reversible oxidation of formate to CO₂ while reducing NAD⁺ to NADH. Formate dehydrogenase, however, also has the unique ability to for example reduce CO₂ while producing formate and/or formic acid. In a specific embodiment, formate dehydrogenase, by the introduction of specific conditions, co-factors or substrates, or specific forms of formate dehydrogenase, may also produce formic acid, formate, or salts thereof and/or a salt thereof such as sodium formate or potassium formate.

In another specific embodiment, the enzymatic reactor cell produces formic acid and/or formate and/or a salt thereof such as sodium formate or potassium formate.

In a specific embodiment, the enzymatic reactor cell has the following formula Ia:

surface—electrode surface linker—formate dehydrogenase (Formula Ia).

In a specific embodiment, Formula I can specifically be as follows:

[surface—electrode surface linker—formate dehydrogenase]ₙ₍₁₋₁₀₎ (Formula Ia);

wherein when n is 2-10, each of the 2-10 [surface—electrode surface linker—enzyme] comprises a different surface, electrode surface linker and/or enzyme from the other [surface—electrode surface linker—enzyme]. In a specific embodiment, at least one of n comprises an enzyme that is a mutant or wildtype form of formate dehydrogenase.

In a specific embodiment, Formula II can specifically be as follows:

[surface—surface linker—enzyme]ₙ₍₁₋₁₀₎ (Formula II);

wherein when n is 2-10, each of the 2-10 [surface—surface linker—enzyme] comprises a different surface, surface linker and/or enzyme from the other [surface—electrode surface linker—enzyme]. In a specific embodiment, at least one of n comprises an enzyme that is a mutant or wildtype form of formate dehydrogenase.

In another specific embodiment, n of Formula I or Formula II is 1, 2, 3, 4, 5, 6, 7, 8, 9, or 10. In another specific embodiment, n of Formula I or Formula II is 11, 12, 13, 14, 15, 16, 17, 18, 19, or 20.

In other words, the reactor cells or a single reactor cell, can include multiple types of surfaces or multiple types of linker or electrode surface linkers, and/or multiple enzymes. For example, a single reactor cell can include more than one enzyme from an enzymatic pathway, wherein the different enzymes in the pathway are attached to the surface of the reactor cell. In a specific embodiment, the different enzymes use different electrode surface linkers and/or different surfaces for a specific linkage for each enzyme, thereby partitioning or separating the different enzymes in the reactor cell as needed. In a specific embodiment, at least one of the reactions or reactor cells comprises a mutant or wildtype form of formate dehydrogenase and produces formic acid and/or formate and/or a salt thereof.

In a specific embodiment, the surface is attached or immobilized to the reactor cell. In another embodiment, the surface is free from and not attached to the reactor cell, such as being free flowing in a solution in the reactor cell. In embodiments, the surface or electrode surface is planar, non-planar, spherical, in the shape of nanoparticles, an aerogel, fibrous, or combinations thereof.

In a specific embodiment, the surface or electrode surface is planar, non-planar, spherical, in the shape of nanoparticles, an aerogel, fibrous, and/or incorporated in a polymer. In a specific embodiment, the surface is a planar surface.

In a specific embodiment, the reactor call may have at least one surface or electrode surface that is an organic or inorganic surface and can be selected based on various advantages, including conductivity, ease of use in a reactor cell, and ease of attachment of the enzyme with the use of a linker. In a specific embodiment, the reactor cell may include more than one type of surface such as a planar surface at one end of the reactor cell and the use of beads in a different portion of the reactor cell.

In another embodiment, the surface or electrode surface of the reactor cell comprises a metal, a polymer, such as polyethyleneimine, perfluorosulfonic acid polymer, or a biological surface. In a specific embodiment, the reactor surface can include at least one surface or electrode surface that comprises one or more selected from the group consisting of graphite, platinum, zinc oxide, cellulose, polystyrene, PDMS,/chlorine doped polypyrrole/polypropylene/etc., gold, gold alloy, gold palladium alloy, and metal oxides such as iron oxide, titanium oxide, manganese oxide, etc. In another specific embodiment, the reactor surface can include at least one surface or electrode surface that comprises one or more selected from the group consisting of various carbon surfaces such as graphite, graphene, carbon paper, or carbon felt, teflonated carbon paper, teflonated carbon felt, carbon based gas diffusion electrode containing a macroporous and/or microporous layer, SWCNT, MWCNT, carbon black, activated carbon, graphite foil, platinum, zinc oxide, cellulose, polystyrene, gold, gold alloy, gold palladium alloy, silver, copper, nickel, zinc, aluminium, stainless steel, alloys, and metal oxides such as iron oxide, titanium, titanium oxide, manganese oxide, silica, etc.

In some embodiments, the surface may include a carbon based surface. In a specific embodiment, the carbon based surface may be Glassy carbon, Carbon/graphite felt, Carbon nanofibers, Carbon nanotubes, Carbon black-coated graphite felt, Microporous carbon, Hierarchically porous carbon, Mesoporous carbon and/or Pyrene (e.g., pyrene loaded to carbon paper or felt). In another embodiment, the surface may be a carbon based electrode and additionally comprise bismuth and/or bismuth oxide. In a specific embodiment, the electrode may be or include carbon paper spray coated with bismuth oxide and carbon black, hydrophilic carbon paper spray coated with bismuth oxide and functionalized carbon nanotubes, and/or hydrophilic carbon paper electrodeposited with bismuth oxide. In another embodiment, the material in the surface may be a carbon based electrode that additionally comprises tin and/or tin oxide. In a specific embodiment, the electrode may include carbon paper spray coated with tin oxide and carbon black; hydrophilic carbon paper spray coated with tin oxide and functionalized carbon nanotubes; and/or hydrophilic carbon paper electrodeposited with tin oxide.

In another embodiment, the surface may be or include a carbon electrode with tin, tin oxide, bismuth, and/or bismuth oxide. In a specific embodiment, the electrode may include or be carbon paper spray coated with tin oxide, bismuth oxide, carbon black; hydrophilic carbon paper spray coated with tin oxide, bismuth oxide, functionalized carbon nanotubes; and/or hydrophilic carbon paper electrodeposited with tin oxide and bismuth oxide.

In another specific embodiment, the surface may be or include a titanium electrode with bismuth and/or bismuth oxide. In a specific embodiment, the electrode may be or include titanium mesh electrodeposited with bismuth oxide, and/or titanium fiber felt electro-deposited with bismuth oxide.

In another specific embodiment, the surface may be or include a titanium electrode with tin and/or tin oxide. In a specific embodiment, the electrode may be or include titanium mesh electrodeposited with tin oxide and/or titanium fiber felt electro-deposited with tin oxide.

In another embodiment, the surface may be or include a titanium electrode with tin, tin oxide, bismuth, and/or bismuth oxide. In a specific embodiment, the electrode may be or include titanium mesh electrodeposited with tin oxide and bismuth oxide and/or titanium fiber felt electro-deposited with tin oxide and bismuth oxide.

In another specific embodiment, the surface may include a polymer such as polyethyleneimine, polyethyleneimine on carbon paper or carbon felt, or other similar polymers known in the art.

In some embodiments, the electrode surface comprises a porous carbon material and/or a carbon nano-construct. In some embodiments, the electrode surface comprises a porous carbon material and/or a carbon nano-construct, and/or a non-biological catalyst. In some embodiments, the non-biological catalyst comprises bismuth, tin, bismuth oxide, or tin oxide. Examples of porous carbon materials include, but are not limited to, carbon paper and carbon felt. Non-limiting examples of carbon nanoparticles include carbon black, carbon nanofibers, and carbon nanotubes. In specific embodiments, the electrode surface comprises glassy carbon, carbon/graphite felt, carbon/graphite paper, carbon nanofibers, carbon nanotubes, carbon black-coated/graphite felt, microporous carbon, hierarchically porous carbon, mesoporous carbon, or pyrene (e.g., pyrene loaded to carbon paper or felt). Pyrene, as used herein, is a molecule capable of increasing surface area, similar to the role of nanoparticles. Depending on the need of use, such as multiple enzymatic steps and pathways, multiple surfaces can be used, for example to attach to linkers that are specific to that surface. Those specific linkers can then also be attached to a specific enzyme. The attachment of the linkers and thus the specific enzymes can then be partitioned based on the surface in the reactor cell.

In a specific embodiment of the reactor cell, the reactor cell may include at least one form of surface attached to an electrode surface linker that is conductive, thereby allowing electron transfer between the enzyme, such as formate dehydrogenase, and the surface or electrode surface. In another specific embodiment the electron transfer travels from formate dehydrogenase to the surface. In another specific embodiment the electron transfer travels from the surface to formate dehydrogenase.

In a specific embodiment, the enzymatic reactor cells can utilize any form of linker as described herein, including a linker that includes a surface binding moiety (SBM) or a material binding peptide, a peptide, protein, a chemical, or polynucleotide. In a specific embodiment, the reactor cells utilize linkers that include a peptide or protein or a protein or peptide nanowire base. In a specific embodiment, these proteins or peptides are conductive and allow for electron transfer between an enzyme and the surface or other intended target in the reactor cell. In a specific embodiment, the linker that allows for electron transfer from the enzyme (such as formate dehydrogenase) and the surface or electrode surface. In a specific embodiment the linkers are electrode surface linkers.

In one embodiment, the use of a peptide or protein in the linker thus allows fusion of the protein, i.e., the linker and the enzyme is a fusion protein, and can be expressed together, thereby allowing a single step of immobilizing the fusion protein to the intended surface.

In a specific embodiment, the reactor cells may comprise cells that allow for linkers to bind to the surface and/or the enzyme formate dehydrogenase by covalent binding, cross-linking and/or entrapment. In a specific embodiment, the linker attaches by click chemistry, dithiol bond formation, Michael addition, a metal-sulfur bond linkage, nucleophilic substitution or enzyme catalyzed conjugation to the surface and/or the enzyme formate dehydrogenase. In a specific embodiment, the linker is covalently or non-covalently attached to formate dehydrogenase. In a specific embodiment, the linker is covalently attached to formate dehydrogenase by continuous protein expression, click chemistry, dithiol bond formation, Michael addition, nucleophilic substitution, a metal-sulfur bond linkage, a metal-nitrogen bond linkage, a metal-oxygen bond linkage, EDC coupling, or enzyme catalyzed conjugation.

In one embodiment, the linker may comprise a chemical nanowire or DNA nanowire type base. In a specific embodiment, the chemical nanowire base can include polymer containing compounds (e.g., MAL-PEG-NHS), cyclic carbon containing compounds (e.g., Succinimidyl 4-(N-maleimidomethyl) cyclohexane-1-carboxylate), or dithiol containing compounds (e.g., 1,6-hexanedithiol). In another specific embodiment, the linker may use, be or include glutaraldehyde nanowires. In another specific embodiment, the linkers or nanowires may, use, be or include SMCC Succinimidyl 4-(N-maleimidomethyl) cyclohexane-1-carboxylate,bis-MAL-dPEG₃, TFP-dPEG₂Mal, MBS (m-maleimidobenzoyl-N-hydroxysuccinimide ester) or a maleimide. In embodiments, the chemical nanowires uses or includes maleimide, TFP-PEGx-Mal, glutaraldehyde, MBS (m-maleimidobenzoyl-N-hydroxysuccinimide ester), bis-MAL-PEGx, C-1,4-phenylene-bis-maleimide, 4,4′-Bis (maleoylamino) azobenzene, ethylene diamine, 6-aminocaproic acid, N,N-ethylene-bis (iodoacetamide), sulfosuccinimidyl (4-iodoacetyl) aminobenzoate, 2-(2-Pyridinyldithio) ethaneamine, dimethyl pimelimidate, 1,4-Butanediol diglycidyl ether, dithiobismaleimidoethane, or 2-iminothiolane.

In a specific embodiment, the nanowire is a PEG based nanowire. In another embodiment, nanowire uses PEG with dithiol such as 1,6-hexanedithiol.

In one embodiment, the linker links by reversible mobilization, wherein the reversible mobilization includes binding by physisorption, bioaffinity, a metal-sulfur bond linkage, and chelation/metal binding. In a specific embodiment, the bioaffinity reversible mobilization is by biotin/streptavidin, or a His-tag. In another embodiment, the linker comprises a binding tag, such as a His tag, a Nickel NTA based tag, or biotin tag or other tag that allows for binding to a specific target, such as the surface or the enzyme or other portion of the linker. In other words, the reactor cells can utilize protein/peptide technology to link to either the surface either covalently or non-covalently such as by high affinity binding moieties that are specific to that surface, or to the enzyme, either covalently (such as a fusion protein) or non-covalently such as by high affinity binding moieties that are specific to that enzyme, or a tag on that enzyme, such as a protein G/antibody or streptavidin/biotin system.

As described herein, the enzymatic reactor cells of the present invention can include one or more enzymes, including oxidoreductase enzymes. The reactor cells may be used for single reaction, using a single enzyme, for example, Example 1-16 described herein. The reactor cells can also be used for multiple reactions, using multiple enzymes in an enzymatic pathway. In a specific embodiment, the pathway can be performed in single reactor cell or more than one reactor cell may be used for the pathway, wherein different enzymes are used in different reactor cells.

Accordingly, one embodiment of the present invention is a system with more than one reactor cells, wherein each reactor cell comprises a different enzymatic reaction or the same enzymatic reaction. In a specific embodiment, the reactor cell comprises one oxidoreductase enzyme and performs a single enzymatic reaction in said enzymatic reactor cell.

In another embodiment, the enzymatic reactor cell comprises more than one oxidoreductase enzyme and performs more than one enzymatic reaction in said enzymatic reactor cell. In another embodiment, the more than one enzymatic reaction in said enzymatic reactor cell is a multi-step pathway. In another specific embodiment, the multi-step pathway is a biological pathway that includes formate dehydrogenase.

In another embodiment, the reactor cells comprise enzymes that are not attached to any surface. In a specific embodiment, the reactor cells may have one or more enzymes that are attached to the surface and another set of enzymes (such as different enzymes in that pathway) that are not attached to the surface. In a specific embodiment, the oxidoreductase enzyme is linked to the electrode surface by the electrode surface linker, and one or more enzymes that are not linked to the surface.

In embodiment of the reactor cells of the present invention, the electrode surface is the cathode, the anode or both. In another specific embodiment, the reactor cells can utilize different electrodes known in the art. For example, the electrode can be made from a carbon-based material such as, for example, CNTs, carbon infiltrated CNTs (CI-CNTs), SWCNTs, MWCNTs, graphene, carbon black, carbon felt, carbon powder, carbon fiber, carbon paper (Toray, ELAT, etc.), pyrolytic carbon, carbon cloth, screen printed carbon, doped diamond, doped diamond-like carbon (DLC), doped polycrystalline diamond (PCD), graphene-coated diamond, DLC, or PCD, and the like.

Electrodes can additionally be made from semiconductive materials, and any semiconductor material capable of electron transport/tunneling to and from the bioelectric material is considered to be within the present scope. Nonlimiting examples of semiconductor materials can include silicon, germanium, which can be doped with elements such as antimony, arsenic, boron, indium, gallium, phosphorus, or combinations thereof.

Electrodes made from conductive metals can also be used in the embodiments of the present disclosure. Any metal or metal alloy material that is electrically conductive, that can support a bioelectric material as described herein, and is capable of enzymes immobilized with a linked or adjacent redox linker or polymer.

Additionally, the present electrodes can be made from metal oxides, metal sulfides, and the like. Any oxide material that is electrically conductive, that can support a bioelectric material as described herein, and is capable of enzymes immobilized in with a linked or adjacent redox linker or polymer is considered to be within the present scope. Nonlimiting examples of oxides can include boron nitride materials, cerium oxide materials, indium-tin oxide (ITO), molybdenum sulfide, titanium oxides, including nanoporous titanium oxide, tin oxides, including tin oxide coated glass, and the like, including combinations thereof.

In another embodiment, the reactor cells can use an electrode such as a 3-electrode cell, flow cell, flow cell with gas diffusion electrode, flow stack cell, flow cell with gas diffusion electrode stack, a rotating disk electrode, a rotating ring disk electrode, a through plane conductivity test set up, or other standard electrochemistry set up. In embodiments, the reactor uses a 3-electrode cell, flow cell, flow cell with gas diffusion electrode, flow stack cell, or a flow cell with gas diffusion electrode stack. In embodiments, the reactor can be used in isolation (e.g., to make formate or formic acid from CO₂ or bicarbonate) or as a part of a series of reactors (e.g., to make other carbon containing chemicals from CO₂).

In another specific embodiment, the enzyme and/or oxidoreductase enzyme are an enzyme and/or oxidoreductase enzymes from a pathway that produces formate, and/or formic acid.

In a specific embodiment, the enzyme reactor cell comprises formate dehydrogenase. In another embodiment, the enzymatic reactor cell produces formate and/or formic acid. In embodiments, the formate ion can be produced in the presence of appropriate counter ions and isolated as the corresponding salt—e.g., sodium to make sodium formate, potassium to make potassium formate, etc.

In another embodiment, the reactor cell may be as depicted in Example 1. In another embodiment the reactor cell(s) may be as depicted in Examples 2-16. In another specific embodiment, the reactor cell or reactor cells may be primed or introduced with a starting material, substrate or any cofactors needed for the enzymatic reaction or pathway to proceed. In a specific embodiment, the reactor cell or cells may initially comprise gaseous carbon dioxide. In another specific embodiment, the reactor cell or cells may initially comprise aqueous carbon dioxide. In another embodiment, the reactor cell or cells may initially comprise bicarbonate. In another embodiment, the reactor cell or cells may initially comprise carbonate. In a specific embodiment, the reactor cell or cells may initially comprise carbonic acid. In a specific embodiment, one reactor cell comprises formate dehydrogenase and performs a single enzymatic reaction in said enzymatic reactor cell.

In one embodiment of the present invention is a system with more than one reactor cell, wherein each reactor cell comprises a different enzymatic reaction or the same enzymatic reaction. In a specific embodiment, the plurality of the enzymatic reactor cells are in a series or are in parallel and at least one enzymatic reactor cell comprises formate dehydrogenase. Accordingly, the present invention may include a plurality of enzymatic reactor cells described herein.

The present invention also includes methods of using the reactor cells and enzymatic pathways described herein.

### Surfaces of the Present Invention

Surfaces of the present invention may include an electrode or non-electrode surface depending on the needs to the enzymatic reaction or enzymatic pathway. In a specific embodiment, the surface is an electrode surface, and can function as either the anode, the cathode, or both in the enzymatic reactor cell. In a specific embodiment, the surface may include graphite, platinum, zinc oxide, cellulose (such as nitrocellulose), polystyrene, PDMS, chlorine doped polypyrrole, polypropylene, polyethyleneimine, perfluorosulfonic acid polymer, gold, gold alloy, gold palladium alloy, and metal oxides such as iron oxide, titanium oxide, manganese oxide, etc., or any combination thereof. In another specific embodiment, the surface may include graphite, platinum, zinc oxide, cellulose (such as nitrocellulose, microcrystalline cellulose, or paper), polystyrene, gold, gold alloy, gold palladium alloy, and metal oxides such as iron oxide, titanium, titanium oxide, manganese oxide, silica, silica coated zinc oxide, etc., or any combination thereof. In another specific embodiment, the reactor surface can include at least one surface or electrode surface that comprises one or more selected from the group consisting of various carbon surfaces such as graphite, graphene, carbon paper, or carbon felt, teflonated carbon paper, teflonated carbon felt, carbon based gas diffusion electrode containing a macroporous and/or microporous layer, SWCNT, MWCNT, carbon black, activated carbon, graphite foil, platinum, zinc oxide, cellulose, polystyrene, gold, gold alloy, gold palladium alloy, titanium, silver, copper, palladium, nickel, zinc, aluminium, stainless steel, and metal oxides such as iron oxide, titanium oxide, manganese oxide, silica, silicon etc. In another specific embodiment, the surface may be titanium, cellulose, polystyrene, carbon surfaces, and/or silica. In a specific embodiment, the surface may allow for a specific binding peptide to bind, wherein the binding peptide is the linker or a portion of the linker. In a specific embodiment, the surface may be for example gold, cellulose, or polystyrene, thereby allowing for affinity binding to a specific binding motif, or a covalent bond to the surface.

In some embodiments, the surface may include a carbon based surface. In a specific embodiment, the carbon based surface may be Glassy carbon, Carbon/graphite felt, Carbon nanofibers, Carbon nanotubes, Carbon black-coated graphite felt, Microporous carbon, Hierarchically porous carbon, Mesoporous carbon and/or Pyrene (e.g., pyrene loaded to carbon paper or felt). In another embodiment, the surface may be a carbon based electrode and additionally comprise bismuth and/or bismuth oxide. In another embodiment, the material in the surface may be a carbon based electrode that additionally comprises tin, bismuth, bismuth oxide and/or tin oxide.

In another specific embodiment, the surface may include a titanium electrode and additionally comprise tin, bismuth, tin oxide, and/or bismuth oxide.

In some embodiments, the electrode surface comprises a porous carbon material and/or a carbon nano-construct. In some embodiments, the electrode surface comprises a porous carbon material and/or a carbon nano-construct, and/or a non-biological catalyst. In some embodiments, the non-biological catalyst comprises bismuth, tin, bismuth oxide, or tin oxide. Examples of porous carbon materials include, but are not limited to, carbon paper and carbon felt. Non-limiting examples of carbon nanoparticles include carbon black, carbon nanofibers, and carbon nanotubes. In specific embodiments, the electrode surface comprises glassy carbon, carbon/graphite felt, carbon/graphite paper, carbon nanofibers, carbon nanotubes, carbon black-coated/graphite felt, microporous carbon, hierarchically porous carbon, mesoporous carbon, or pyrene (e.g., pyrene loaded to carbon paper or felt). Pyrene, as used herein, is a molecule capable of increasing surface area, similar to the role of nanoparticles.

In another embodiment, the surfaces may be in various shapes or forms to facilitate immobilization of the enzyme and/or the reaction as desired. For example, the surface may be planar, non-planar, spherical, in the shape of nanoparticles, in the shape of beads, an aerogel, fibrous, or be or include a polymer. In a specific embodiment, the surface may be planar allowing for one or more types of enzymes to be immobilized to the surface. In a specific embodiment, the surface may be in the form of nanoparticles. In another embodiment, the nanoparticles may be silica coated zinc oxide nanoparticles.

### Linkers of the Present Invention

Linkers may be included in the present invention. In one or more embodiment, the linkers of the present invention may have one or more of the following features: 1) a means for linking to the surface; 2) a means for linking to the enzyme, thereby providing a link between the surface and the enzyme; and 3) the linker is, or comprises a portion of the linker that allows for electron transfer between the enzyme (such as an oxidoreductase enzyme) and the surface or electrode surface. In a specific embodiment, the linker allows for electron transfer from the enzyme to the surface or from the surface to the enzyme.

In a specific embodiment the linkers are electrode surface linkers and are conductive. In a specific embodiment, the electrode surface linkers allow for electron transfer from the oxidoreductase enzyme and the surface or electrode surface. In one embodiment, the linker binds on one end to the surface, and the other end to the enzyme, thereby immobilizing the enzyme to the surface. In a specific embodiment, the linker is an electrode surface linker, thereby allowing electron transfer from the enzyme to the surface, or vice-versa. In a specific embodiment, the electrode surface linker is conductive, thereby allowing electron transfer from the oxidoreductase enzyme and the electrode surface.

In one embodiment, the linker or electrode surface linker binds to the surface.

In one embodiment, the linker or electrode surface linker comprises a surface binding moiety (SBM). In a specific embodiment, the surface binding moiety provide a means for binding to the surface, which may include various methods, including irreversible and reversible immobilization of the linker and enzyme to the surface. Irreversible immobilization includes covalent binding, cross-linking and entrapment, while reversible methods include physisorption, bioaffinity (biotin/streptavidin and protein A/G), chelation/metal binding, a metal-sulfur bond linkage, and disulfide bonds (LIÉBANA; DRAGO, 2016).

In a specific embodiment, the linker is an electrode surface linker linked to the surface or electrode surface by irreversible or reversible immobilization. In another embodiment, the surface binding moiety (SBM) is immobilized to the surface or electrode surface by irreversible mobilization, wherein the irreversible mobilization is by covalent binding, cross-linking and/or entrapment. In a specific embodiment the surface binding moiety (SBM) is covalently bonded to the electrode surface. In another embodiment, the surface binding moiety (SBM) attaches by click chemistry, dithiol bond formation, Michael addition (for example, the use of an acrylamide linkage), a metal-sulfur bond linkage, nucleophilic substitution or enzyme catalyzed conjugation. In another embodiment, the immobilization or binding may be by physiosorption.

In another embodiment, the surface binding moiety (SBM) may be or include a material binding protein or peptide. In a specific embodiment, the linker or electrode surface linker comprises a peptide for binding to the surface.

In another embodiment, the linker or electrode surface linker comprises a gold binding motif or binding peptide, a graphite/graphene binding motif or binding peptide, a PDMS binding motif or peptide, a silica binding motif or binding peptide, a cellulose binding motif or peptide, a polystyrene binding motif or peptide, a zinc oxide binding motif or binding peptide, a platinum binding motif or binding peptide, or a AuPd binding motif or binding binding peptide. In another embodiment, the linker or electrode surface linker comprises a copper binding motif or binding peptide, a palladium binding motif or binding peptide, a nickel binding motif or binding peptide, a zinc binding motif or binding peptide, an aluminium binding motif or binding peptide, a stainless steel binding motif or binding peptide, an iron oxide binding motif or binding peptide, a titanium oxide binding motif or binding peptide, a polypyrrole or a chlorine doped polypyrrole binding motif or binding peptide, or a polypropylene binding motif or binding peptide,. In another embodiment, the linker or electrode surface linker comprises a titanium binding motif or binding peptide. In a specific embodiment, the surface binding moiety of the linkers may comprise one or more of the material binding peptide (MBP) amino acid sequences from Table 1.

TABLE 1
MBP Sequences

| | | | | Molecular | | |
|---|---|---|---|---|---|---|
| | | | Length | weight | | SEQ ID |
| ID | Target | Peptide Sequence | (aa) | (Dalton) | Reference | NO: |
| EMT014 | Gold | MHGKTQATSGTIQSMHG | 42 | 4303.752 | (KULP; | SEQ ID |
| | | KTQATSGTIQSMHGKTQ | | | SARIKAYA; | NO: 1 |
| | | ATSGTIQS | | | EVANS, | |
| | | | | | 2004) | |
| EMT015 | Gold | MHGKTQATSGTIQSMHG | 98 | 10018.0684 | (BROWN, | SEQ ID |
| | | KTQATSGTIQSMHGKTQ | | | 1997) | NO: 2 |
| | | ATSGTIQSMHGKTQATS | | | | |
| | | GTIQSMHGKTQATSGTIQ | | | | |
| | | SMHGKTQATSGTIQSMH | | | | |
| | | GKTQATSGTIQS | | | | |
| EMT016 | Gold | WAGAKRLVLRRE | 12 | 1454.7371 | (HNILOVA; | SEQ ID |
| | | | | | OREN; | NO: 3 |
| | | | | | SEKER; | |
| | | | | | WILSON | |
| | | | | | et al., | |
| | | | | | 2008) | |
| EMT017 | Gold | HFSSWETQQG | 10 | 1206.2336 | (TANAKA; | SEQ ID |
| | | | | | HIKIBA; | NO: 4 |
| EMT018 | Gold | WYEKWQKANW | 10 | 1438.6042 | YAMAS | SEQ ID |
| | | | | | HITA; | NO: 5 |
| | | | | | MUTO | |
| | | | | | et al., | |
| | | | | | 2017) | |
| EMT019 | Gold | VSGSSPDS | 8 | 734.716 | (HUANG; | SEQ ID |
| | | | | | CHIANG; | NO: 6 |
| | | | | | LEE; | |
| | | | | | GAO et | |
| | | | | | al., | |
| | | | | | 2005) | |
| EMT020 | Silicon | SSKKSGSYSGSKGSRRIL | 36 | 3541.8909 | (KRÖGER; | SEQ ID |
| | | GGGGMHGKTQATSGTIQ | | | DEUTZMANN; | NO: 7 |
| | | S | | | SUMPER, | |
| | | | | | 1999) | |
| EMT021 | Silicon | MSPHPHPRHHHTGGGGM | 30 | 3127.4165 | (NAIK; | SEQ ID |
| | | HGKTQATSGTIQS | | | BROTT; | NO: 8 |
| EMT022 | Silicon | RGRRRRLSCRLLGGGGM | 30 | 3198.6687 | CARSON; | SEQ ID |
| | | HGKTQATSGTIQS | | | AL., | NO: 9 |
| | | | | | 2012) | |
| EMT023 | Silicon | DSARGFKKPGKRGGGG | 30 | 3003.3374 | (COYLE; | SEQ ID |
| | | | | | BANEYX, | NO: 10 |
| | | MHGKTQATSGTIQS | | | 2016) | |
| EMT024 | Silicon | HPPMNASHPHMHGGGG | 30 | 3049.3582 | (ETESHOLA; | SEQ ID |
| | | MHGKTQATSGTIQS | | | BRILLSON; | NO: 11 |
| | | | | | LEE, | |
| | | | | | 2005) | |
| EMT025 | Silicon | HKDHHANQHVHMGGGG | 30 | 3147.4045 | (OKAMOTO; | SEQ ID |
| | | MHGKTQATSGTIQS | | | IWAHORI; | NO: 12 |
| | | | | | YAMAS | |
| | | | | | HITA, | |
| | | | | | 2019) | |
| EMT026 | Silicon | HPPMNASHPHMHGGGG | 16 | | | SEQ ID |
| | | | | | | NO: 13 |
| Cellulose | Cellulose | PTTGSCAVTYTANGWSG | 108 | | | SEQ ID |
| binding | | GFTAAVTLTNTGTTALSG | | | | NO: 14 |
| motif 1 | | WTLGFAFPSGQTLTQGW | | | | |
| | | SARWAQSGSSVTATNEA | | | | |
| | | WNAVLAPGASVEIGFSG | | | | |
| | | THTGTNTAPATFTVGGA | | | | |
| | | TCTTR | | | | |
| Cellulose | Cellulose | SGPAGCQVLWGVNQWN | 108 | | | SEQ ID |
| binding | | TGFTANVTVKNTSSAPV | | | | NO: 15 |
| motif 2 | | DGWTLTFSFPSGQQVTQ | | | | |
| | | AWSSTVTQSGSAVTVRN | | | | |
| | | APWNGSIPAGGTAQFGF | | | | |
| | | NGSHTGTNAAPTAFSLN | | | | |
| | | GTPCTVG | | | | |
| Polystyrene | Polystyrene | RAFIASRRIRRP | 12 | | | SEQ ID |
| binding | | | | | | NO: 16 |
| motif 1 | | | | | | |
| Polystyrene | Polystyrene | RIIIRRIR | 9 | | | SEQ ID |
| binding | | | | | | NO: 17 |
| motif 2 | | | | | | |
| Silica | Silica | RGRRRRLSCRLL | 12 | | | SEQ ID |
| Binding | | | | | | NO: 18 |
| Motif 1 | | | | | | |
| Silica | Silica | SSKKSGSYSGSKGSRRIL | 18 | | | SEQ ID |
| Binding | | | | | | NO: 19 |
| Motif 2 | | | | | | |
| Silica | Silica | MSPHPHPRHHHT | 12 | | | SEQ ID |
| Binding | | | | | | NO: 20 |
| Motif 3 | | | | | | |
| Silica | Silica | DSARGFKKPGKR | 12 | | | SEQ ID |
| Binding | | | | | | NO: 21 |
| Motif 4 | | | | | | |
| Silica | Silica | HPPMNASHPHMH | 12 | | | SEQ ID |
| Binding | | | | | | NO: 22 |
| Motif 5 | | | | | | |
| Silica | Silica | HKDHHANQHVHM | 12 | | | SEQ ID |
| Binding | | | | | | NO: 23 |
| Motif 6 | | | | | | |
| Silver | Silver | NPSSLFRYLPSD | 12 | | | SEQ ID |
| Binding | | | | | | NO: 24 |
| Motif 1 | | | | | | |
| Graphene | Graphene | HSSYWYAFNNKT | 12 | | | SEQ ID |
| Binding | | | | | | NO: 25 |
| Motif | | | | | | |
| Zinc | Zinc Oxide | EAHVMHKVAPRP | 12 | | | SEQ ID |
| Oxide | | | | | | NO: 26 |
| Binding | | | | | | |
| Motif 1 | | | | | | |
| Zinc | Zinc Oxide | HHGHSPTSPQVR | 12 | | | SEQ ID |
| Oxide | | | | | | NO: 27 |
| Binding | | | | | | |
| Motif 2 | | | | | | |
| PDMS | PDMS | MVMPGDNIKMVVTLIHPI | 30 | | | SEQ ID |
| Binding | (Polydimet | AMDDGLRFAIRE | | | | NO: 28 |
| Motif 1 | hylsiloxane) | | | | | |
| PDMS | PDMS | VGPNNVPYIVATITSNSA | 45 | | | SEQ ID |
| Binding | (Polydimet | GGQPVSLANLKAMYSIA | | | | NO: 29 |
| Motif 2 | hylsiloxane) | KKYDIPVVMD | | | | |
| PDMS | PDMS | NNSWTRVAFAGLKFQDV | 31 | | | SEQ ID |
| Binding | (Polydimet | GSFDYGRNYGVVYD | | | | NO: 30 |
| Motif 3 | hylsiloxane) | | | | | |
| Gold | Gold 1 | QVQLVESGAEVKKPGES | 120 | | | SEQ ID |
| binding | | LKISCKGSGYSFPSYWIN | | | | NO: 31 |
| motif 1 | | WVRQMPGKGLEWMGMI | | | | |
| | | YPADSDTRYSPSFQGHVT | | | | |
| | | ISADKSINTAYLQWAGLK | | | | |
| | | ASDTAIYYCARLGIGGRY | | | | |
| | | MSRWGQGTLVTVSSA | | | | |
| Platinum | Platinum | LEYKRGYKPR | 10 | | | SEQ ID |
| binding | | | | | | NO: 32 |
| motif 1 | | | | | | |
| AuPd | AuPd | DYKDDDDKAYSS | 20 | | | SEQ ID |
| binding | | GAPPMPPF | | | | NO: 33 |
| motif 1 | | | | | | |
| Ti binding | Titanium | RKLPDA | 6 | | | SEQ ID |
| motif 1 | | | | | | NO: 50 |
| Graphite/ | Graphite | EPLQLKM | 7 | | | SEQ ID |
| graphene | | | | | | NO: 54 |
| binding | | | | | | |
| peptide 1 | | | | | | |
| Graphite/ | Graphite | GAMHLPWHMGTL | 12 | | | SEQ ID |
| graphene | | | | | | NO: 55 |
| binding | | | | | | |
| peptide 2 | | | | | | |
| Graphite/ | Graphite | IMVTASSAYRRY | 12 | | | SEQ ID |
| graphene | | | | | | NO: 56 |
| binding | | | | | | |
| peptide 3 | | | | | | |
| Graphite/ | Graphite | VIAGASLWWSEKLVIA | 16 | | | SEQ ID |
| graphene | | | | | | NO: 57 |
| binding | | | | | | |
| peptide 4 | | | | | | |
| Graphite/ | Graphite | CALNNDEVDKFAM | 13 | | | SEQ ID |
| graphene | | | | | | NO: 58 |
| binding | | | | | | |
| peptide 5 | | | | | | |
| Graphite/ | Graphite | KKNYSSSISSIHC | 13 | | | SEQ ID |
| graphene | | | | | | NO: 59 |
| binding | | | | | | |
| peptide 6 | | | | | | |
| Graphite/ | Graphite | CGGHSSKLQFWYFWY | 15 | | | SEQ ID |
| graphene | | | | | | NO: 60 |
| binding | | | | | | |
| peptide 7 | | | | | | |
| Graphite/ | Graphite | HWSAWWIRSNQS | 12 | | | SEQ ID |
| graphene | | | | | | NO: 61 |
| binding | | | | | | |
| peptide 8 | | | | | | |
| Graphite/ | Graphite | IMVTESSDYSSY | 12 | | | SEQ ID |
| graphene | | | | | | NO: 62 |
| binding | | | | | | |
| peptide 9 | | | | | | |
| Graphite/ | Graphite | IMVTASSAYDDY | 12 | | | SEQ ID |
| graphene | | | | | | NO: 63 |
| binding | | | | | | |
| peptide 10 | | | | | | |
| Graphite/ | Graphite | IMVTQSSNYSSY | 12 | | | SEQ ID |
| graphene | | | | | | NO: 64 |
| binding | | | | | | |
| peptide 11 | | | | | | |
| Graphite/ | Graphite | IMVTKSSDYSSY | 12 | | | SEQ ID |
| graphene | | | | | | NO: 65 |
| binding | | | | | | |
| peptide 12 | | | | | | |
| Graphite/ | Graphite | HSSAAAAFNNKT | 12 | | | SEQ ID |
| graphene | | | | | | NO: 66 |
| binding | | | | | | |
| peptide 13 | | | | | | |
| Graphite/ | Graphite | HTSYWYAFNTKT | 12 | | | SEQ ID |
| graphene | | | | | | NO: 67 |
| binding | | | | | | |
| peptide 14 | | | | | | |
| Graphite/ | Graphite | YTTHVLPFAPSS | 12 | | | SEQ ID |
| graphene | | | | | | NO: 68 |
| binding | | | | | | |
| peptide 15 | | | | | | |
| Graphite/ | Graphite | HAWVDWIRPIH | 11 | | | SEQ ID |
| graphene | | | | | | NO: 69 |
| binding | | | | | | |
| peptide 16 | | | | | | |
| Graphite/ | Graphite | HWKHPWGAWDTL | 12 | | | SEQ ID |
| graphene | | | | | | NO: 70 |
| binding | | | | | | |
| peptide 17 | | | | | | |
| Graphite/ | Graphite | QQQLSTH | 7 | | | SEQ ID |
| graphene | | | | | | NO: 71 |
| binding | | | | | | |
| peptide 18 | | | | | | |
| Graphite/ | Graphite | GGPDSARGFKKPGKR | 18 | | | SEQ ID |
| graphene | | GPC | | | | NO: 72 |
| binding | | | | | | |
| peptide 19 | | | | | | |
| Cellulose | cellulose | GGGMHPNAGHGSLMR | 15 | | | SEQ ID |
| binding | | | | | | NO: 73 |
| peptide 1 | | | | | | |
| CBD_{CBH1} | cellulose | TPQSHYGQCGGGYSGPT | 36 | | | SEQ ID |
| | | VCASGTTCQVLNPYYSQ | | | | NO: 74 |
| | | CL | | | | |
| CBD_{ClpC} | | GVVSVQFNNGSSPASSNS | 157 | | | SEQ ID |
| | | IYARFKVTNTSGSPINLA | | | | NO: 75 |
| | | DLKLRYYYTQDADKPLT | | | | |
| | | FWCDHAGYMSGSNYID | | | | |
| | | ATSKVTGSFKAVSPAVT | | | | |
| | | NADHYLEVALNSDAGSL | | | | |
| | | PAGGSIEIQTRFARNDWS | | | | |
| | | NFDQSNDWSYTAAGSY | | | | |
| | | MDWQKISAFVGGTLAYG | | | | |
| | | STP | | | | |
| Ti binding | Titanium | QPYLFATDSLIK | 12 | | | SEQ ID |
| motif 2 | | | | | | NO: 76 |
| Ti binding | Titanium | GHTHYHAVRTQT | 12 | | | SEQ ID |
| motif 3 | | | | | | NO: 77 |
| Polystyrene | Polystyrene | RLLLRRLRR | 9 | | | SEQ ID |
| binding | | | | | | NO: 78 |
| motif 3 | | | | | | |
| Polystyrene | Polystyrene | KRAFIASRRIRRP | 13 | | | SEQ ID |
| binding | | | | | | NO: 79 |
| motif 4 | | | | | | |
| GSH | Copper | ECG | 3 | | (Slocik; | SEQ ID |
| | | | | | Wright, | NO: 80 |
| | | | | | 2003) | |
| HG12 | Copper | HGGGHGHGGGHG | 12 | | (Banerjee; | SEQ ID |
| | | | | | Yu; | NO: 81 |
| | | | | | Matsui, | |
| | | | | | 2003) | |
| Pd4 | Palladium | TSNAVHPTLRHL | 12 | | (Heinz; | SEQ ID |
| | | | | | Farmer; | NO: 82 |
| | | | | | Pandey; | |
| | | | | | Slocik et | |
| | | | | | al., | |
| | | | | | 2005) | |
| Pd2 | Palladium | NFMSLPRLGHMH | 12 | | (Heinz; | SEQ ID |
| | | | | | Farmer; | NO: 83 |
| | | | | | Pandey; | |
| | | | | | Slocik et | |
| | | | | | al., | |
| | | | | | 2005) | |
| HRE | Nickel | AHHAHHAAD | 9 | | (Slocik; | SEQ ID |
| | | | | | Wright, | NO: 84 |
| | | | | | 2003) | |
| SMN01 | Nickel | SGTGASY | 7 | | (Braun; | SEQ ID |
| | | | | | Bachmann; | NO: 85 |
| | | | | | Schonberger; | |
| | | | | | Matys et | |
| | | | | | al., | |
| | | | | | 2018) | |
| | Nickel | DAHKSEVA | 8 | | (Bar-Or; | SEQ ID |
| | | | | | Curtis; | NO: 86 |
| | | | | | Rao; | |
| | | | | | Bampos | |
| | | | | | et al., | |
| | | | | | 2001) | |
| | Zinc | SYHHHH | 6 | | (Mejare; | SEQ ID |
| | | | | | Bulow, | NO: 87 |
| | | | | | 2001) | |
| | Zinc | ERSWTLDSALSM | 12 | | (Roth- | SEQ ID |
| | | | | | enstein; | NO: 88 |
| | | | | | Claasen; | |
| | | | | | Omiecienski; | |
| | | | | | Lammel | |
| | | | | | et al., | |
| | | | | | 2012) | |
| A1-S1 | Aluminium | VPSSGPQDTRTT | 12 | | (Zuo; | SEQ ID |
| | | | | | Orken; | NO: 89 |
| | | | | | Wood, | |
| | | | | | 2005) | |
| A1-S2 | Aluminium | YSPDPRPWSSRY | 12 | | (Zuo; | SEQ ID |
| | | | | | Orken; | NO: 90 |
| | | | | | Wood, | |
| | | | | | 2005) | |
| MS-S1 | Stainless | NLNPNTASAMHV | 12 | | (Cao; | SEQ ID |
| | steel | | | | Yuan; | NO: 91 |
| | | | | | Ma; | |
| | | | | | Yang et | |
| | | | | | al., 2015) | |
| | Stainless | VQHNTKYSVVIR | 12 | | (Zuo; | SEQ ID |
| | steel | | | | Orken; | NO: 92 |
| | | | | | Wood, | |
| | | | | | 2005) | |
| SBP-A | Stainless | VQHNTKYSVVIR | 12 | | (Mikami; | SEQ ID |
| | steel | | | | Fujimoto; | NO: 93 |
| | | | | | Taguchi; | |
| | | | | | Isao et | |
| | | | | | al., | |
| | | | | | 2020) | |
| | Stainless | MTWDPSLASPRS | 12 | | (Vreuls; | SEQ ID |
| | steel | | | | Zocchi; | NO: 94 |
| | | | | | Genin; | |
| | | | | | Archambeau | |
| | | | | | et al., | |
| | | | | | 2010) | |
| K122-4 | Stainless | ACTSNADNKYLPKTCQT | 17 | | (Muruve; | SEQ ID |
| | steel | | | | Cheng; | NO: 95 |
| | | | | | Feng; | |
| | | | | | Liu et | |
| | | | | | al., | |
| | | | | | 2016) | |
| 1 | Iron Oxide | RRTVKHHVN | 9 | | (Sarikaya; | SEQ ID |
| | | | | | Tamerler; | NO: 96 |
| | | | | | Jen; | |
| | | | | | Schulten | |
| | | | | | et al., | |
| | | | | | 2003) | |
| | Iron Oxide | QMDTSTSLAPSR | 12 | | (Lower; | SEQ ID |
| | | | | | Lins; | NO: 97 |
| | | | | | Oestreicher; | |
| | | | | | Straatsma, | |
| | | | | | 2008) | |
| HRE | Titanium | AHHAHHAAD | 9 | | (Slocik; | SEQ ID |
| | Oxide | | | | Wright, | NO: 98 |
| | | | | | 2003) | |
| TiC1 | Titanium | CHKKPSKSC | 9 | | (Chen; | SEQ ID |
| | Oxide | | | | Su; | NO: 99 |
| | | | | | Neoh; | |
| | | | | | Choe, | |
| | | | | | 2006) | |
| φT59 | Chlorine | THRTSTLDYFVI | 12 | | (Sanghvi; | SEQ ID |
| | Doped | | | | Miller; | NO: 100 |
| | Polypyrrole | | | | Belcher; | |
| | | | | | Schmidt, | |
| | | | | | 2005) | |
| | Poly- | TSDIKSRSPHHR | 12 | | PATENT | SEQ ID |
| | propylene | | | | (Cunningham; | NO: 101 |
| | | | | | Lowe; | |
| | | | | | O'brien; | |
| | | | | | Wang et | |
| | | | | | al., | |
| | | | | | 2011) | |
| | Poly- | HTQNMRMYEPWF | 12 | | PATENT | SEQ ID |
| | propylene | | | | (Cunningham; | NO: 102 |
| | | | | | Lowe; | |
| | | | | | O'brien; | |
| | | | | | Wang et | |
| | | | | | al., | |
| | | | | | 2011) | |
| LCI | Poly- | AIKLVQSPNGNFAASFVL | 47 | | (Rubsam; | SEQ ID |
| | propylene | DGTKWIFKSKYYDSSKG | | | Weber; | NO: 103 |
| | | YWVGIYEVWDRK | | | Jakob; | |
| | | | | | Schwaneberg, | |
| | | | | | 2017) | |
| LCI-M1- | Poly- | AIKLVQSPNGNFAASFVL | 47 | | (Rubsam; | SEQ ID |
| PP | propylene | DGTKWTFKSKHYDSSKG | | | Weber; | NO: 104 |
| | | YWVGIYKVWDRK | | | Jakob; | |
| | | | | | Schwaneberg, | |
| | | | | | 2017) | |
| KR-2 | Poly- | AIKLVQSPNGNFAASFVL | 47 | | (Rubsam; | SEQ ID |
| | propylene | DGTKWIFKSKRYDSSKG | | | Davari; | NO: 105 |
| | | YWVGIYEVWRRK | | | Jakob; | |
| | | | | | Schwaneberg, | |
| | | | | | 2018) | |
| | SnO | LPPWKLK | | | | SEQ ID |
| | | | | | | NO. 124 |
| | SnO | WSLSELH | | | | SEQ ID |
| | | | | | | NO. 125 |
| | SnO | IGASVHR | | | | SEQ ID |
| | | | | | | NO. 126 |
| | SnO | AHHLKVS | | | | SEQ ID |
| | | | | | | NO. 127 |
| | SnO | NHPLYNR | | | | SEQ ID |
| | | | | | | NO. 128 |
| | SnO | ALEHTSR | | | | SEQ ID |
| | | | | | | NO. 129 |
| | SnO | HPAIRPP | | | | SEQ ID |
| | | | | | | NO. 130 |
| | SnO | LHRHANL | | | | SEQ ID |
| | | | | | | NO. 131 |
| | SnO | SSNQFHQ | | | | SEQ ID |
| | | | | | | NO. 132 |
| | SnO | KVPGHQQ | | | | SEQ ID |
| | | | | | | NO. 133 |
| | SnO | TLAPRTA | | | | SEQ ID |
| | | | | | | NO. 134 |
| | SnO | VGKTHAD | | | | SEQ ID |
| | | | | | | NO. 135 |
| | SnO | FPLHELR | | | | SEQ ID |
| | | | | | | NO. 136 |
| | SnO | LPPW | | | | SEQ ID |
| | | | | | | NO. 137 |
| | SnO | WKLK | | | | SEQ ID |
| | | | | | | NO. 138 |
| | Tin oxide | KNAGQYPPSALM | | | | SEQ ID |
| | | | | | | NO. 139 |
| | Tin oxide | SPSHSADHTPPT | | | | SEQ ID |
| | | | | | | NO. 140 |
| | Tin oxide | TPTLRSMSSLLF | | | | SEQ ID |
| | | | | | | NO. 141 |
| | Tin oxide | STLTQSTSSLVA | | | | SEQ ID |
| | | | | | | NO. 142 |
| | SnO₂ | INTKNYALRTLH | | | | SEQ ID |
| | | | | | | NO. 143 |
| | SnO₂ | KLHISKDHIYPT | | | | SEQ ID |
| | | | | | | NO. 144 |
| | SnO₂ | SWMPHPRRSPGH | | | | SEQ ID |
| | | | | | | NO. 145 |
| | SnO₂ | SSYYPGLTAHRF | | | | SEQ ID |
| | | | | | | NO. 146 |
| | SnO₂ | GAMHLPWHMGTL | | | | SEQ ID |
| | | | | | | NO. 147 |
| | SnO₂ | VRCIIRGIVGTH | | | | SEQ ID |
| | | | | | | NO. 148 |
| | SnO₂ | LGCLIRVGVLQH | | | | SEQ ID |
| | | | | | | NO. 149 |
| | SnO₂ | YTKPTENIHQRA | | | | SEQ ID |
| | | | | | | NO. 150 |
| | SnO₂ | WSRIIRQASYTQ | | | | SEQ ID |
| | | | | | | NO. 151 |
| | SnO₂ | TYAKGHHSWPPP | | | | SEQ ID |
| | | | | | | NO. 152 |
| | SnO₂ | SGMPHPRRSPQH | | | | SEQ ID |
| | | | | | | NO. 153 |
| | SnO₂ | TIMGETFAMPAS | | | | SEQ ID |
| | | | | | | NO. 154 |
| | SnO₂ | ETLHISTHPNET | | | | SEQ ID |
| | | | | | | NO. 155 |
| | SnO₂ | GIGLIRGRSQEL | | | | SEQ ID |
| | | | | | | NO. 156 |
| | SnO₂ | LLWVRLLLCSAS | | | | SEQ ID |
| | | | | | | NO. 157 |
| | SnO₂ | GGMHIPWHMGPL | | | | SEQ ID |
| | | | | | | NO. 158 |
| | SnO₂ | GGKHNPRHTDPR | | | | SEQ ID |
| | | | | | | NO. 159 |
| | SnO₂ | KHMHNLTPQWTK | | | | SEQ ID |
| | | | | | | NO. 160 |
| | SnO₂ | SGHHNLHKTEHR | | | | SEQ ID |
| | | | | | | NO. 161 |
| | SnO₂ | GHHHSKTFETQT | | | | SEQ ID |
| | | | | | | NO. 162 |
| | SnO₂ | GHSMHRPLKLFH | | | | SEQ ID |
| | | | | | | NO. 163 |
| | SnO₂ | GLSMHRPLKLFH | | | | SEQ ID |
| | | | | | | NO. 164 |
| | SnO₂ | KPHHNWESAYSK | | | | SEQ ID |
| | | | | | | NO. 165 |
| | SnO₂ | VMNHWQEDLMFGY | | | | SEQ ID |
| | | | | | | NO. 166 |
| | SnO₂ | PENLLYYHPGTD | | | | SEQ ID |
| | | | | | | NO. 167 |
| | SnO₂ | TGTPAMVDANNG | | | | SEQ ID |
| | | | | | | NO. 168 |
| | SnO₂ | YSENEEIVGLAAK | | | | SEQ ID |
| | | | | | | NO. 169 |
| | SnO₂ | TCFFRDHSYQEE | | | | SEQ ID |
| | | | | | | NO. 170 |
| | SnO₂ | IDEITDINNT | | | | SEQ ID |
| | | | | | | NO. 171 |

In another embodiment, the present invention may comprise an amino acid sequence as in Table 1. In another embodiment, the present invention includes nucleotide sequence of about 75% to about 99.9% identical to one or more of SEQ ID NOs. 1-33, 50 or 54-105. In another embodiment, the present invention includes a nucleotide sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID NOs. 1-33, 50 or 54-105.

In another embodiment, the present invention includes nucleotide sequence of about 75% to about 99.9% identical to one or more of SEQ ID NOs. 124-171. In another embodiment, the present invention includes a nucleotide sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID NOs. 124-171. In another embodiment, electrode surface linker comprises a protein wherein the peptide or protein is a protein or peptide nanowire base.

In a specific embodiment, the MBP is for binding to SnO, Tin oxide, and/or SnO₂.

The linkers may be conductive and may allow for electron transfer. In a specific embodiment, the linker that allows for electron transfer between the enzyme (such as an oxidoreductase enzyme) and the surface or electrode surface. In a specific embodiment the linkers are electrode surface linkers. In another specific embodiment, linker allows for electron transfer from the enzyme (such as an oxidoreductase enzyme) to the surface or electrode surface and/or for electron transfer from the surface to the enzyme.

In one embodiment, the linker may comprise a chemical nanowire, a means for chemical linkage, or a DNA nanowire type base. In a specific embodiment, the chemical nanowire base can include polymer containing compounds (e.g., MAL-PEG-NHS), cyclic carbon containing compounds (e.g., Succinimidyl 4-(N-maleimidomethyl) cyclohexane-1-carboxylate), or dithiol containing compounds (e.g., 1,6-hexanedithiol), or a means for a a metal-sulfur bond linkage. In embodiments, the chemical nanowires uses or includes maleimide, TFP-PEGx-Mal, glutaraldehyde, MBS (m-maleimidobenzoyl-N-hydroxysuccinimide ester), bis-MAL-PEGx, C-1,4-phenylene-bis-maleimide, 4,4′-Bis (maleoylamino) azobenzene, ethylene diamine, 6-aminocaproic acid, N,N-ethylene-bis (iodoacetamide), sulfosuccinimidyl (4-iodoacetyl) aminobenzoate, 2-(2-Pyridinyldithio) ethaneamine, dimethyl pimelimidate, 1,4-Butanediol diglycidyl ether, dithiobismaleimidoethane, or 2-iminothiolane.

In embodiments, the linker may comprise a chemical based nanowire which comprises a maleimide functional group, a tetrafluorophenyl functional group, an aldehyde functional group, an amine functional group, a N-hydroxysuccinimide functional group, a thiol functional group, a haloacetyl functional group, a pyridyl disulfide functional group, an imidoester functional group, an epoxide functional group, a hydrocarbon chain, one of more polyethylene glycol units, or one or more aromatic rings or a derivative thereof. For example, the chemical based nanowire may comprise maleimide, TFP-PEGx-Mal, glutaraldehyde, MBS (m-maleimidobenzoyl-N-hydroxysuccinimide ester), bis-MAL-PEGx/maleimide, C-1,4-phenylene-bis-maleimide, 4,4′-Bis (maleoylamino) azobenzene/ethylene diamine, 6-aminocaproic aci, N,N-ethylene-bis (iodoacetamide), sulfosuccinimidyl (4-iodoacetyl) aminobenzoate, 2-(2-Pyridinyldithio) ethaneamine, dimethyl pimelimidate, 1,4-Butanediol diglycidyl ether, dithiobismaleimidoethane, or 2-iminothiolane.

In another specific embodiment, the linker may use, be, or include glutaraldehyde nanowires. In another specific embodiment, the nanowires may, use, be, or include SMCC Succinimidyl 4-(N-maleimidomethyl) cyclohexane-1-carboxylate, MBS (m-maleimidobenzoyl-N-hydroxysuccinimide ester) or a maleimide. In a specific embodiment, chemical modification may be used to link two portions of the linker or a portion of the linker to the surface, or a portion of the linker to the enzyme.

In another embodiment, the linker may comprise a DNA nanowire base, such as double stranded DNA, such as DNA composed of G and C bases ranging in length from about 4.08 nm to about 16 μm. In another embodiment, the linker may comprise a DNA nanowire base, such as double stranded DNA, such as DNA composed of G and C bases ranging in length from about 0.1 nm to about 10 μm or any length therebetween. In another embodiment, the length is from about 1.00 nm to about 1.0 μm. In another embodiment, the length is from about 1.00 nm to about 10 nm. In another embodiment, the length is from about 1.5 nm to about 5 nm. In another embodiment, the length is from about 1.00 nm to about 3 nm. In another embodiment, the length is about 1.00 nm, 2 nm, 3 nm, 4 nm, 5 nm, 6 nm, 7 nm, 8 nm, 9 nm, or 10 nm. In another embodiment, the DNA is single stranded DNA, such as single stranded DNA composed of G and C bases ranging in length from about 4.08 nm to about 16 μm. In another embodiment, the DNA is single stranded DNA, such as single stranded DNA composed of G and C bases ranging in length from about 0.1 nm to about 10 μm or any length therebetween. In another embodiment, the length is from about 1.00 nm to about 1.0 μm. In another embodiment, the length is from about 1.00 nm to about 10 nm. In another embodiment, the length is from about 1.5 nm to about 5 nm. In another embodiment, the length is from about 1.00 nm to about 3 nm. In another embodiment, the length is about 1.00 nm, 2 nm, 3 nm, 4 nm, 5 nm, 6 nm, 7 nm, 8 nm, 9 nm, or 10 nm.

In another embodiment, the DNA of the nanowire base is a polynucleotide of at least 2, 3, 4, 5, 6, 7, 8, 9, 10, 11, 12, 13, 14, or 15 nucleotides in length. In another embodiment, the polynucleotide sequences are single stranded or double stranded. In another embodiment, the nanowires comprise a nucleotide sequence of Table 3. In a specific embodiment, the nucleotide sequence may be any of the single stranded sequences of Table 3 or together as a double stranded sequence such as, for example, a double stranded sequence comprising SEQ ID Nos: 52 and 53.

TABLE 3
Nanowire Polynucleotide Sequences

| | Nucleotide Sequence | |
|---|---|---|
| ID | 5′ to 3′ | SEQ ID NO: |
| SSDNA1 | GGATGC | SEQ ID NO: 52 |
| SSDNA2 | GCATCC | SEQ ID NO: 53 |
| SSDNA3 | GCAACTAGGCTCG | SEQ ID NO: 106 |
| SSDNA4 | CGAGCCTAGTTGC | SEQ ID NO: 107 |
| SSDNA5 | GCACCTGAACCGCATGGACTCG | SEQ ID NO: 108 |
| SSDNA6 | CGAGTCCATGCGGTTCAGGTGC | SEQ ID NO: 109 |

In another embodiment, the present invention may comprise a nucleotide as in Table 3. In another embodiment, the present invention includes nucleotide sequence of about 75% to about 99.9% identical to one or more of SEQ ID Nos 52-53 or SEQ ID Nos 106-109. In another embodiment, the present invention includes a nucleotide sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID Nos 52-53 or SEQ ID Nos 106-109.

In another embodiment, the linker includes a protein or peptide nanowire base that includes a material binding peptide, a short conductive peptide, a metal-sulfur bond linkage, and/or one or more ferredoxins. In embodiments, the protein or peptide nanowire base comprises an iron-sulfur cluster containing conductive protein/peptide e.g., ferredoxin like structures/cytochrome like structures etc. In another embodiment, the one or more ferredoxins comprises a chain of ferredoxins. In another specific embodiment, the protein nanowire comprises ferredoxins, with 1 FeS cluster, more than 1 FeS cluster, a chain of ferrodoxins with 1 FeS clusters, a chain of ferrodoxins with more than 1 FeS clusters, or a chain of ferredoxins that includes a combination of ferredoxins with 1 FeS cluster and ferredoxins with >1 FeS cluster.

In another embodiment, the ferredoxin is from Chlamydomonas reinhardtii and/or Clostridium pasteurianum, in one or more clusters or chains as described above. In another embodiment, the linker or electrode surface linker may comprise one or more conductive proteins from E. coli. In a specific embodiment, the E. coli is E. coli C321, and the linker is the fimA protein. In another embodiment, the fimA protein is wildtype or a mutant. In a specific embodiment, the fimA is a mutant and point mutations at the following residues: A80, A109, and/or H82. In a specific embodiment, the mutations may be as follows: E. coli C321 fimA protein with A80F point mutation; E. coli C321 fimA protein with A109F point mutation; E. coli C321 fimA protein with A80F and A109F point mutation; E. coli C321 fimA protein with A80F, H82F and A109F point mutation; E. coli C321 fimA protein with A80F, H82F and A109Y point mutation; E. coli C321 fimA protein with A80Y point mutation; E. coli C321 fimA protein with A109Y point mutation; E. Coli C321 fimA protein with A80Y and A109Y point mutations; E. coli C321 fimA protein with A80W point mutation; E. coli C321 fimA protein with A109W point mutation; E. coli C321 fimA protein with A80W and A109W point mutations; E. coli C321 fimA protein with A80Y, H82F and A80Y point mutations; E. coli C321 fimA protein with A80Y, H82Y and A109Y point mutations; or E. coli C321 fimA protein with a A109 2NaA point mutation.

In another embodiment, the linker or electrode surface linker may comprise one or more conductive proteins from pili/archaelum (e.g., from Syntrophus aciditrophicus, Methanospirillum hungatei, Geobacter metallireducens) or other conductive extracellular proteins (e.g., OmcS cytochrome, OmcZ cytochrome).

In embodiments, the linker comprises one or more short conductive peptides known in the art, including one or more conductive peptides with the amino acid sequence as in Table 2.

In another embodiment, the linkers comprise one or more short conductive peptides known in the art, including one or more conductive peptides with the amino acid sequence as in Table 2.

TABLE 2
Conductive Peptide Sequences

| ID | Peptide Sequence | SEQ ID NO: |
|---|---|---|
| Conductive | FTLIELLIVVAIIGILAAIAIPQFSAYRVKAYN | SEQ ID NO: 34 |
| Peptide 1 | SAASSDLRNLKTALESAFADDQTYPPES | |
| Conductive | FTLIELMIVVAIIGILAAIAIPQYQNYVARSEG | SEQ ID NO: 35 |
| Peptide 2 | ASALASVNPLKTTVEEALSRGWSVKSGTGTEDA | |
| | TKKEVPLGVAADANKLGTIALKPDPADGTADIT | |
| | LTFTMGGAGPKNKGKIITLTRTAADGLWKCTSD | |
| | QDEQFIPKGCSR | |
| Conductive | MASNFKFKLLSQLKKRAEGGFTLIELLVVVIII | SEQ ID NO: 36 |
| Peptide 3 | GVLAAIALPNLLGQVGKARESEAKSTIGALNRA | |
| | QQGYFTEKGTFATDTETLEVPAPDGNFFSFAVN | |
| | TADNTEAIQDATALNWEADGTRSMSGGTFYDSG | |
| | TRAFSTVVCRAEAGSEDTPPTPGGANDCGGAEV | |
| | IK | |
| Conductive | ELKAIAQEFKAIAKEFAIAFEFKAIAQK | SEQ ID NO: 37 |
| Peptide 4 | | |
| Conductive | YYACAYY | SEQ ID NO: 38 |
| Peptide 5 | | |
| Conductive | GNNQQNY | SEQ ID NO: 39 |
| Peptide 6 | | |
| Conductive | KVQIINKKL | SEQ ID NO: 40 |
| Peptide 7 | | |
| Conductive | VGGLG | SEQ ID NO: 41 |
| Peptide 8 | | |
| Conductive | VGGLGHHH | SEQ ID NO: 42 |
| Peptide 9 | | |
| Conductive | VGGLGWWW | SEQ ID NO: 43 |
| Peptide 10 | | |
| Conductive | VGGLGYYY | SEQ ID NO: 44 |
| Peptide 11 | | |
| Conductive | VGGLFFF | SEQ ID NO: 45 |
| Peptide 12 | | |
| Conductive | SVNVTQVGFP | SEQ ID NO: 46 |
| Peptide 13 | | |
| Conductive | SVNVTQVGFPHHH | SEQ ID NO: 47 |
| Peptide 14 | | |
| Conductive | SVNVTQVGFPWWW | SEQ ID NO: 48 |
| Peptide 15 | | |
| Conductive | SVNVTQVGFPYYY | SEQ ID NO: 49 |
| Peptide 16 | | |
| Conductive | PPPYPPPWC | SEQ ID NO: 51 |
| peptide 17 | | |
| Nanowire 1 | (GS)ₙ (n = 1-8) | SEQ ID NO: 172 |
| Nanowire 2 | VDGGGGGS | SEQ ID NO: 173 |
| Nanowire 3 | GTGGGGVDGGGGGS | SEQ ID NO: 174 |
| Nanowire 4 | GGGG | SEQ ID NO: 175 |
| Nanowire 5 | GGGGG | SEQ ID NO: 176 |
| Nanowire 6 | GGGGGG | SEQ ID NO: 177 |
| Nanowire 7 | GGGGGGGG | SEQ ID NO: 178 |
| Nanowire 8 | (GGS)ₙ (n = 1-5) | SEQ ID NO: 179 |
| Nanowire 9 | (GGGS)ₙ (n = 1-4) | SEQ ID NO: 180 |
| Nanowire 10 | PAPAP | SEQ ID NO: 181 |
| Nanowire 11 | (EAAAK)ₙ (n = 1-4) | SEQ ID NO: 182 |
| Nanowire 12 | GGYGG | SEQ ID NO: 183 |
| Nanowire 13 | GGFGG | SEQ ID NO: 184 |
| Nanowire 14 | GGFFGG | SEQ ID NO: 185 |
| Nanowire 15 | GGYYGG | SEQ ID NO: 186 |
| Nanowire 16 | GGSYWGGS | SEQ ID NO: 187 |
| Nanowire 17 | GGGGS(EAAAK)ₙGGGS (n = 1, 2) | SEQ ID NO: 188 |
| Nanowire 18 | (GGGGS)ₙ (n = 1-4) | SEQ ID NO: 206 |

In another embodiment, the present invention may comprise a peptide or an amino acid sequence as in Table 2. In another specific embodiment, the peptide consists of the amino acid sequences as in Table 2. In another embodiment, the present invention includes a peptide or an amino acid sequence of about 75% to about 99.9% identical to one or more of SEQ ID Nos 34-51. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID Nos 34-51.

In another embodiment, the present invention includes a peptide or an amino acid sequence of about 75% to about 99.9% identical to one or more of SEQ ID Nos 172-188 and 206. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID Nos 172-188 and 206. In a specific embodiment, the peptide or amino acid sequence is a nanowire, which can be fused to an enzyme and/or other portions of the linker. In another embodiment, the nanowire may be GS.

In another embodiment, the present invention includes a peptide or an amino acid sequence of about 75% to about 99.9% identical to SEQ ID No. 49. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 49.

In another embodiment, the present invention includes a peptide or an amino acid sequence of about 75% to about 99.9% identical to SEQ ID No. 48. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 48.

In another embodiment, the present invention includes a peptide or an amino acid sequence of about 75% to about 99.9% identical to SEQ ID No. 47. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 47.

In another embodiment, the present invention includes a peptide or an amino acid sequence of about 75% to about 99.9% identical to SEQ ID No. 51. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 51.

The linkers and electrode surface linkers of the present invention may also include any appropriate amino acid sequence required to control steric hindrance and/or chemical interactions (organic or inorganic materials, peptides and proteins, cross-linking reagents, etc.).

The linker sequences of the present invention may include one or more passive linkers and/or active linkers. In certain embodiments, a passive linker is fused to an active linker, e.g., to link the SBM to the active portion of the linker. As used herein, a passive linker does not specifically bind to the surface or enzyme and is typically present between two polypeptide sequences to control steric hindrance, e.g., to retain activity of the two linked polypeptides. In particular embodiments, a passive linker may be a single bond or an amino acid sequence that links the SBM to another portion of the linker or directly to the enzyme. A passive linker may also be present between the enzyme and a member of a binding pair to which it is fused. The linker may be a covalent bond, an ionic bond, a non-covalent bond such as with the use of high-affinity molecules.

As used herein, an active linker may be fused to the SBM and/or enzyme and may be present to functionally link the SBM to the enzyme. In particular embodiments, an active linker binds to the enzyme. In certain embodiments, an active linker is a member of a binding pair, such as streptavidin/biotin. The link may be a covalent bond, an ionic bond, a non-covalent bond such as with the use of high-affinity molecules.

In another embodiment, the linker sequence may include other amino acid sequences, such as passive linkers, a linear tandem repeat polypeptides, a linear non-repeating polypeptides or linkers that allow for additional flexibility or rigidity to the overall linker system.

In one embodiment, the linker or electrode surface linker binds to the enzyme or an oxidoreductase enzyme.

As described above the linker or electrode surface linker may comprise a surface binding moiety (SBM) that binds to the surface. The SBM may then also bind to a separate portion of the linker, e.g., a passive portion or active portion of the linker, or to the enzyme directly.

The linker or electrode surface linker thus provides a means for binding to the enzyme such as formate dehydrogenase, which may include various methods, including irreversible and reversible immobilization of the linker to the enzyme. Irreversible immobilization includes covalent binding, cross-linking and entrapment, while reversible methods include random physisorption, bioaffinity (biotin/streptavidin and protein A/G), a metal-sulfur bond linkage, chelation/metal binding and disulfide bonds (LIÉBANA; DRAGO, 2016).

In a specific embodiment, the linker is an electrode surface linker linked to the enzyme such as formate dehydrogenase by irreversible or reversible immobilization. In another embodiment, the linker, including potentially the SBM of the linker, is immobilized to the enzyme by irreversible mobilization, wherein the irreversible mobilization is by covalent binding, cross-linking and/or entrapment. In a specific embodiment the surface binding moiety (SBM) or another portion of the linker is covalently bonded to the enzyme. In another embodiment, the surface binding moiety (SBM) or another portion of the linker is covalently attached by click chemistry, dithiol bond formation, Michael addition (for example, the use of an acrylamide linkage), a metal-sulfur bond linkage, nucleophilic substitution, a metal-nitrogen bond linkage, a metal-oxygen bond linkage, enzyme catalyzed conjugation, or EDC coupling.

The link to the enzyme may be a covalent bond, an ionic bond, a non-covalent bond such as with the use of high-affinity molecules. In embodiments, the linker is covalently or non-covalently attached to the enzyme. For example, in embodiments, the linker is covalently attached to the enzyme by continuous protein expression, click chemistry, dithiol bond formation, Michael addition, nucleophilic substitution, a metal-sulfur bond linkage, a metal-nitrogen bond linkage, a metal-oxygen bond linkage, EDC coupling, or enzyme catalyzed conjugation. In embodiments, the linker is non-covalently attached to the enzyme by high affinity binding.

In a specific embodiment, the high affinity molecule in the linker may be an amino acid sequence comprising protein G from Streptococcus, or an amino acid sequence comprising streptavidin from Streptomyces for binding to the enzyme. In another embodiment, the linker comprises a binding tag, such as a His tag, a Nickel NTA based tag, or biotin tag or other tag that allows for binding to the enzyme. In another embodiment, the tags may be on the enzyme, and therein the linker comprises the portion for binding to the tag. In other words, the linker may comprise streptavidin or biotin or the enzyme may comprise biotin or streptavidin respectively.

### Enzymes and Enzymatic Pathways of the Present Invention

Enzymatic bioelectrocatalysis is a specific form of electrocatalysis using an enzyme for catalyzing a certain reaction. In one general example of enzymatic bioelectrocatalysis, enzymes are associated with an electrode, including electrode linkers, in a manner that allows electron transfer between the electrode and the enzymes. Such electron transfer allows the continued function of each enzyme over many catalyzed reactions, including a series of reaction to obtain a final desired compound or product. The term “enzyme(s)” as used herein thus relates to a biologically based catalytic mechanism, and can comprise a protein that is both wild-type or mutated for any intended reaction by the user. Nonlimiting examples of other biologically based catalytic materials can include eukaryotic cells, prokaryotic cells, cellular organelles, nucleic acid enzymes (i.e. deoxyribozymes), and the like.

Oxidoreductase enzymes are biocatalytic proteins that can catalyse the coupled oxidation and reduction with a substrate, thus, transferring an electron(s) with the involvement of an electrode linker and/or a cofactor of the enzyme. In a specific embodiment of the present invention, the oxidoreductase enzyme can be used in a single reaction or in a series of reactions with other enzymes or oxidoreductase enzymes wherein the oxidoreductase enzyme is directly linked to an electrode linker. In a specific embodiment, the enzyme or oxidoreductase enzyme is directly linked to an electrode linker in a device, biodevice or reactor cell that contains the reaction or series of reactions if more than one enzyme or oxidoreductase enzyme is used.

In a specific embodiment, the enzyme can be an oxidoreductase enzyme. In another embodiment, the enzyme and/or oxidoreductase enzyme is one of a set or multiple enzymes and/or oxidoreductase enzymes. In another embodiment, the set of or multiple enzymes and/or oxidoreductase enzymes or apart of an enzymatic pathway.

Formate dehydrogenase is an enzyme that is traditionally known to catalyze the reversible oxidation of formate to CO₂ while reducing NAD⁺ to NADH. Formate dehydrogenase, however, also has the unique ability to, for example, reduce CO₂ while producing formic acid. In a specific embodiment, formate dehydrogenase, by the introduction of specific conditions, co-factors or substrates, or specific forms of formate dehydrogenase, may also produce formic acid, formate, or salts thereof, such as sodium formate or potassium formate.

In a specific embodiment, the oxidoreductase enzyme comprises formate dehydrogenase. In one embodiment, the enzymes, whether are formate dehydrogenase or not, are provided to produce formate and/or formic acid. This can be achieved by the addition of specific mutants to the enzyme and/or by the introduction of specific conditions, co-factors or substrates to achieve such production of formate and/or formic acid. In a specific embodiment, the enzyme such as formate dehydrogenase as mentioned above may be wild-type. In another specific embodiment, the enzyme, such as formate dehydrogenase may be a mutant. In another embodiment, the enzymes may have one or more amino acid residues mutated to a cysteine residue. In another specific embodiment, the mutation to a cysteine residue is on the outer surface of the folded enzyme, thereby allowing easy access to binding or reactive groups for linking to the thiol of the cysteine residue.

In embodiments, the enzyme is formate dehydrogenase (formate: NAD+ oxidoreductase (EC 1.17.1.9) or formate: ferricytochrome-b1 oxidoreductase (EC 1.2.2.1)).

In embodiments, the enzyme is formate dehydrogenase with mutation(s) to introduce new functional groups near the surface of the enzyme (eg., cysteine, lysine, etc.).

In embodiments, the enzyme is formate dehydrogenase with mutations to enhance the size, activity, or stability of the enzyme.

In embodiments, the enzyme is wildtype or mutant Formate dehydrogenase without metal centers: e.g., from Myceliophthora thermophila/Candida boidinii/Thiobacillus sp./etc.

In embodiments, the enzyme is wildtype or mutant formate dehydrogenase with metal centers: e.g., from Rhodobacter capsulatus/Desulfovibrio vulgaris/Cupriavidus necator/Clostridium ljundahlii/Paraclostridium bifermentansetc.

In a specific embodiment, the enzyme may be that of any sequence in Table 4.

TABLE 4
Enzyme Sequences

| ID | AA Sequence | SEQ ID NO: |
|---|---|---|
| Formate | MHHHHHHENLYFQMKIVLVLYDAGKHAADEEK | SEQ ID NO: 110 |
| dehydrogenase | LYGCTENKLGIANWLKDQGHELITTSDKEGGN | |
| With N | SVLDQHIPDADIIITTPFHPAYITKERIDKAK | |
| terminal His | KLKLVVVAGVGSDHIDLDYINQTGKKISVLEV | |
| Tag | TGSNVVSVAEHVLMTMLVLVRNFVPAHEQIIN | |
| Candida | HDWEVAAIAKDAYDIEGKTIATIGAGRIGYRV | |
| boidinii | LERLVPFNPKELLYYDYQALPKDAEEKVGARR | |
| | VENIEELVAQADIVTINAPLHAGTKGLINKEL | |
| | LSKFKKGAWLVNTARGAICVAEDVAAALESGQ | |
| | LRGYGGDVWFPQPAPKDHPWRDMRNKYGAGNA | |
| | MTPHYSGTTLDAQTRYAEGTKNILESFFTGKF | |
| | DYRPQDIILLNGEYITKAYGKHDKK | |
| Formate | MKIVLVLYDAGKHAADEEKLYGCTENKLGIAN | SEQ ID NO: 111 |
| dehydrogenase | WLKDQGHELITTSDKEGGNSVLDQHIPDADII | |
| Candida | ITTPFHPAYITKERIDKAKKLKLVVVAGVGSD | |
| boidinii | HIDLDYINQTGKKISVLEVTGSNVVSVAEHVL | |
| | MTMLVLVRNFVPAHEQIINHDWEVAAIAKDAY | |
| | DIEGKTIATIGAGRIGYRVLERLVPFNPKELL | |
| | YYDYQALPKDAEEKVGARRVENIEELVAQADI | |
| | VTINAPLHAGTKGLINKELLSKFKKGAWLVNT | |
| | ARGAICVAEDVAAALESGQLRGYGGDVWFPQP | |
| | APKDHPWRDMRNKYGAGNAMTPHYSGTTLDAQ | |
| | TRYAEGTKNILESFFTGKFDYRPQDIILLNGE | |
| | YITKAYGKHDKK | |
| Formate | MKIVLVLYDAGKHAADEEKLYGCTENKLGIAN | SEQ ID NO: 112 |
| dehydrogenase | WLKDQGHELITTSDKEGGNSVLDQHIPDADII | |
| with His Tag on | ITTPFHPAYITKERIDKAKKLKLVVVAGVGSD | |
| C terminal end | HIDLDYINQTGKKISVLEVTGSNVVSVAEHVL | |
| Candida | MTMLVLVRNFVPAHEQIINHDWEVAAIAKDAY | |
| boidinii | DIEGKTIATIGAGRIGYRVLERLVPFNPKELL | |
| | YYDYQALPKDAEEKVGARRVENIEELVAQADI | |
| | VTINAPLHAGTKGLINKELLSKFKKGAWLVNT | |
| | ARGAICVAEDVAAALESGQLRGYGGDVWSPQP | |
| | APKDHPWRDMRNKYGAGNAMTPHYSGTTLDAQ | |
| | TRYAEGTKNILESFFTGKFDYRPQDIILLNGE | |
| | YITKAYGKHDKKHHHHHH | |
| Formate | MAHHHHHHVGMVKVLAVLYDGGKHAQQVPGLL | SEQ ID NO: 113 |
| dehydrogenase | GTTENELGLRKWLEDQGHTLVTTSDKDGENST | |
| from | FDRELVDAEIIITTPFHPGYLTAERLAKAKKL | |
| Myceliophthora | KLAVTAGIGSDHVDLDAANKINGGITVAEVTG | |
| thermophila | SNVVSVAEHVVMTILVLVRNFVPAHEQIEAGR | |
| with His-tag | WDVAEAAKNEFDLEGKVVGTVAVGRIGERVLR | |
| | RLKAFDCKELLYYDYQPLSPEKEKEIGCRRVL | |
| | DLEEMLGQCDVVTINCPLHEKTRGLFNKDLIA | |
| | KMKPGSWLVNTARGAIVVKEDVAEALRTGHLR | |
| | GYGGDVWFPQPAPADHPLRTAKNPFGGGNAMV | |
| | PHMSGTSLDAQKRYADGVKRILESYLSGRFDY | |
| | RPEDLIVHNGQYATRSYGQREVSTS | |
| Formate | MAVGMVKVLAVLYDGGKHAQQVPGLLGTTENE | SEQ ID NO: 114 |
| dehydrogenase | LGLRKWLEDQGHTLVTTSDKDGENSTFDRELV | |
| from | DAEIIITTPFHPGYLTAERLAKAKKLKLAVTA | |
| Myceliophthora | GIGSDHVDLDAANKTNGGITVAEVTGSNVVSV | |
| thermophila | AEHVVMTILVLVRNFVPAHEQIEAGRWDVAEA | |
| without His-tag | AKNEFDLEGKVVGTVAVGRIGERVLRRLKAFD | |
| | CKELLYYDYQPLSPEKEKEIGCRRVLDLEEML | |
| | GQCDVVTINCPLHEKTRGLFNKDLIAKMKPGS | |
| | WLVNTARGAIVVKEDVAEALRTGHLRGYGGDV | |
| | WFPQPAPADHPLRTAKNPFGGGNAMVPHMSGT | |
| | SLDAQKRYADGVKRILESYLSGRFDYRPEDLI | |
| | VHNGQYATRSYGQREVSTS | |
| Formate | MAKILCVLYDDPVDGYPKTYARDDLPKIDHYP | SEQ ID NO: 115 |
| dehydrogenase | GGQTLPTPKAIDFTPGQLLGSVSGELGLRKYL | |
| from | EANGHTFVVTSDKDGPDSVFEKELVDADVVIS | |
| Thiobacillus sp. | QPFWPAYLTPERIAKAKNLKLALTAGIGSDHV | |
| with His-tag | DLQSAIDRGITVAEVTYCNSISVAEHVVMMIL | |
| | GLVRNYIPSHDWARKGGWNIADCVEHSYDLEG | |
| | MTVGSVAAGRIGLAVLRRLAPFDVKLHYTDRH | |
| | RLPEAVEKELGLVWHDTREDMYPHCDVVTLNV | |
| | PLHPETEHMINDETLKLFKRGAYIVNTARGKL | |
| | ADRDAIVRAIESGQLAGYAGDVWFPQPAPKDH | |
| | PWRTMKWEGMTPHISGTSLSAQARYAAGTREI | |
| | LECFFEGRPIRDEYLIVQGGALAGTGAHSYSK | |
| | GNATGGSEEAAKFKKAGHHHHHH | |
| Formate | MAKILCVLYDDPVDGYPKTYARDDLPKIDHYP | SEQ ID NO: 116 |
| dehydrogenase | GGQTLPTPKAIDFTPGQLLGSVSGELGLRKYL | |
| from | EANGHTFVVTSDKDGPDSVFEKELVDADVVIS | |
| Thiobacillus sp. | QPFWPAYLTPERIAKAKNLKLALTAGIGSDHV | |
| without His-tag | DLQSAIDRGITVAEVTYCNSISVAEHVVMMIL | |
| | GLVRNYIPSHDWARKGGWNIADCVEHSYDLEG | |
| | MTVGSVAAGRIGLAVLRRLAPFDVKLHYTDRH | |
| | RLPEAVEKELGLVWHDTREDMYPHCDVVTLNV | |
| | PLHPETEHMINDETLKLFKRGAYIVNTARGKL | |
| | ADRDAIVRAIESGQLAGYAGDVWFPQPAPKDH | |
| | PWRTMKWEGMTPHISGTSLSAQARYAAGTREI | |
| | LECFFEGRPIRDEYLIVQGGALAGTGAHSYSK | |
| | GNATGGSEEAAKFKKAG | |
| Formate | MHHHHHHENLYFQGSMTDTARLRAILAAHRGR | SEQ ID NO: 117 |
| dehydrogenase | EGALLPILHDVQAAFGFIPEDAYAPIAADLGL | |
| from | TRAEVAGVVGFYHDFRKAPAGRHVIKLCRAEA | |
| Rhodobacter | CQAMGMDAVQARLESALGLRLGDSSEAVTLEA | |
| capsulatus | VYCLGLCACAPAAMVDDRLVGRLDAAAVAGIV | |
| With His-tag | AELGA | |
| fdsG with His- | | |
| tag | | |
| Formate | MKIWLPCDAAAKACGAEAVLAALRLEAEKRGG | SEQ ID NO: 118 |
| dehydrogenase | ALDIARNGSRGMIWLEPLLEVETPAGRIGFGP | |
| from | MTPADVPALFDALESHPKALGLVEEIPFFKRQ | |
| Rhodobacter | TRLTFARCGRIEPLSLAQFAAAEGWAGLRKAL | |
| capsulatus | KMTPAEVVEEVLASGLRGRGGAGFPTGIKWRT | |
| fdsB | VAAAQADQKYIVCNVDEGDSGSFADRMLIEGD | |
| | PFCLVEGMAIAGHAVGATRGYVYIRSEYPDAI | |
| | AVMRAAIAMAKPFLAEAGFEMEVRVGAGAYVC | |
| | GEETSLLNSLEGKRGTVRAKPPLPALKGLFGK | |
| | PTVVNNLLSLAAVPWIIAHGAKAYESFGMDRS | |
| | RGTIPLQIGGNVKRGGLFETGFGITLGELVED | |
| | ICGGTASGRPVKAVQVGGPLGAYHPVSDYHLP | |
| | FCYEQFAGQGGLVGHAGLVVHDDTADMLKLAR | |
| | FAMEFCAIESCGTCTPCRIGAVRGVEVIDRIA | |
| | AGDASAMPLLDDLCQTMKLGSLCALGGFTPYP | |
| | VQSAIRHFPADFPCAREAAE | |
| Formate | MKDLIIPPLDWTQDMGTPKREGAPVHLTIDGV | SEQ ID NO: 119 |
| dehydrogenase | EVTVPAGTSVLRAAAEAGISIPKLCATDNVEP | |
| from | VGSCRLCMVEIEGMRGTPTSCTTPVAPGMRVH | |
| Rhodobacter | TQTPQLQKLRRGVMELYISDHPLDCLTCAANG | |
| capsulatus | DCELQDMAGAVGLREVRYQAKDTHFARRDATG | |
| fdsA | PNPRYIPKDNSNPYFSYDPAKCIVCMRCVRAC | |
| | EEVQGTFALTVMGRGFDARISPAAPDFLSSDC | |
| | VSCGACVQACPTATLVEKSVERIGTPERKVVT | |
| | TCAYCGVGCSFEAHMLGDQLVRMVPWKGGAAN | |
| | RGHSCVKGRFAYGYATHQDRILKPMIRDKITD | |
| | PWREVNWTEALDFTATRLRALRDSHGADALGV | |
| | ITSSRCTNEETYLVQKLARAVFGTNNTDTCAR | |
| | VCHSPTGYGLKQTFGTSAGTQDFDSVEETDLA | |
| | LVIGANPTDGHPVFASRLRKRLRAGAKLIVVD | |
| | PRRIDLLNTPHRGEAWHLQLKPGTNVAVMTAM | |
| | AHVIVTEQIFDKRFIGDRCDWDEWADYAEFVA | |
| | NPEYAPEAVESLTGVPAGLLRQAARAYAAAPN | |
| | AAIYYGLGVTEHSQGSTTVIAIANLAMMTGNI | |
| | GRPGVGVNPLRGQNNVQGSCDMGSFPHEFPGY | |
| | RHVSDDATRGLFERTWGVTLSSEPGLRIPNML | |
| | DAAVEGRFKALYVQGEDILQSDPDTRHVSAGL | |
| | AAMDLVIVHDLFLNETANYAHVFLPGSTFLEK | |
| | DGTFTNAERRINRVRRVMAPKAGFADWEVTQM | |
| | LANALGAGWHYTHPSEIMAEIAATTPGFAAVT | |
| | YEMLDARGSVQWPCNEKAPEGSPIMHVEGFVR | |
| | GKGRFIRTAYLPTDEKTGPRFPLLLTTGRILS | |
| | QYNVGAQTRRTENTVWHGEDRLEIHPTDAETR | |
| | GIRDGDWVRLASRAGETTLRATVTDRVSPGVV | |
| | YTTFHHPDTQANVVTTDTSDWATNCPEYKVTA | |
| | VQVAASNGPSDWQQDYAAQAAAARRIEAAE | |
| Formate | MSLPAGAVTVPLPGGARAVLAEEVPVALVFDG | SEQ ID NO: 120 |
| dehydrogenase | VTQAVMMASPVDLEDFLLGFALTEGMIADRAE | |
| from | LLRHEVVRQPQGIELRGWLAAPAGQRFAARRR | |
| Rhodobacter | AMAGPVGCGLCGLDSLAAVLRPLPRAPRGGAP | |
| capsulatus | PPLADGALAALRAGQSLQDAVRSVHAAGFWDG | |
| fdsC | AQMRALREDVGRHNALDKLAGALAGQGIDAAA | |
| | GALVLTSRLSVDLVQKAAMIGARVLIAPSAPT | |
| | ALAVAEAQAAGLALIARGPDGPTLYTETEAE | |
| Formate | MSDDKIIRMANQIAAFFAVQPGDRAGPVAAHI | SEQ ID NO: 121 |
| dehydrogenase | SENWSAPMRAALLAHVAAQSPGLDPLVIAAAP | |
| from | QIRPVPALEHHHHHH | |
| Rhodobacter | | |
| capsulatus | | |
| fdsD with His- | | |
| tag | | |
| Formate | MGSMTDTARLRAILAAHRGREGALLPILHDVQ | SEQ ID NO: 122 |
| dehydrogenase | AAFGFIPEDAYAPIAADLGLTRAEVAGVVGFY | |
| from | HDFRKAPAGRHVIKLCRAEACQAMGMDAVQAR | |
| Rhodobacter | LESALGLRLGDSSEAVTLEAVYCLGLCACAPA | |
| capsulatus | AMVDDRLVGRLDAAAVAGIVAELGA | |
| Without His-tag | | |
| fdsG | | |
| Formate | MSDDKIIRMANQIAAFFAVQPGDRAGPVAAHI | SEQ ID NO: 123 |
| dehydrogenase | SENWSAPMRAALLAHVAAQSPGLDPLVIAAAP | |
| from | QIRPVPALE | |
| Rhodobacter | | |
| capsulatus | | |
| fds-D without | | |
| HisTag | | |
| Candida | MKIVLVLYDCGKHAADEEKLYGCTENKLGIAN | SEQ ID NO: 189 |
| boidinii formate | WLKDQGHELITTSDKEGGNSVLDQHIPDADII | |
| dehydrogenase | ITTPFHPAYITKERIDKAKKLKLVVVAGVGSD | |
| with a C | HIDLDYINQTGKKISVLEVTGSNVVSVAEHVL | |
| terminal His- | MTMLVLVRNFVPAHEQIINHDWEVAAIAKDAY | |
| tag. | DIEGKTIATIGAGRIGYRVLERLVPFNPKELL | |
| | YYDYQALPKDAEEKVGARRVENIEELVAQADI | |
| | VTINAPLHAGTKGLINKELLSKFKKGAWLVNT | |
| | ARGAICVAEDVAAALESGQLRGYGGDVWSPQP | |
| | APKDHPWRDMRNKYGAGNAMTPHYSGTTLDAQ | |
| | TRYAEGTKNILESFFTGKFDYRPQDIILLNGE | |
| | YITKAYGKHDKKHHHHHH | |
| Candida | MAHHHHHHKIVLVLYDCGKHAADEEKLYGCTE | SEQ ID NO: 190 |
| boidinii formate | NKLGIANWLKDQGHELITTSDKEGGNSVLDQH | |
| dehydrogenase | IPDADIIITTPFHPAYITKERIDKAKKLKLVV | |
| with a C | VAGVGSDHIDLDYINQTGKKISVLEVTGSNVV | |
| terminal | SVAEHVLMTMLVLVRNFVPAHEQIINHDWEVA | |
| nanowire (GS), | AIAKDAYDIEGKTIATIGAGRIGYRVLERLVP | |
| C terminal | FNPKELLYYDYQALPKDAEEKVGARRVENIEE | |
| MBP (SEQ ID | LVAQADIVTINAPLHAGTKGLINKELLSKFKK | |
| No. 25), and N | GAWLVNTARGAICVAEDVAAALESGQLRGYGG | |
| terminal His- | DVWSPQPAPKDHPWRDMRNKYGAGNAMTPHYS | |
| tag. | GTTLDAQTRYAEGTKNILESFFTGKFDYRPQD | |
| | IILLNGEYITKAYGKHDKKGSHSSYWYAFNNK | |
| | T | |
| Candida | MKIVLVLYD(C/A)GKHAADEEKLYG(C/S)T | SEQ ID NO: 191 |
| boidinii formate | ENKLGIANWLKDQGHELITTSDKEGGNSVLDQ | |
| dehydrogenase | HIPDADIIITTPFHPAYITKERIDKAKKLKLV | |
| with a C | VVAGVGSDHIDLDYINQTGKKISVLEVTGSNV | |
| terminal His- | (V/C)SVAEHVLMTMLVLVRNFVPAHEQIINH | |
| tag and one or | DW(E/C)VAAIAKDAYDIEGKTIATIGAGRIG | |
| more of the | YRVLERLVPFNPKELLYYDYQALP(K/C)DAE | |
| following | EKVGARRVENIEELVAQADIVTINAPLHAGTK | |
| mutations: | GLIN(K/C)ELLSKFKKGAWLVNTARGAICVA | |
| a: V121C | EDVAAAL(E/C)(S/C)GQLRGYGGDVWSPQP | |
| b: E151C | APKDHPWRDMRNKYGAGNAMTPHYSGTTL | |
| c: K201C | (D/C)AQ(T/C)RYAEGTKNILESFFTGKFDY | |
| d: K241C | RPQDIILLNGEYITKAYGKHDKKHHHHHH | |
| e: E272C | | |
| f: S273C | | |
| g: D318C | | |
| h: T321C | | |
| k: V121C/C10A/ | | |
| C23S | | |
| L: E151C/C10A/ | | |
| C23S | | |
| m: K201C/C10A/ | | |
| C23S | | |
| n: K241C/C10A/ | | |
| C23S | | |
| o: E272C/C10A/ | | |
| C23S | | |
| p: S273C/C10A/ | | |
| C23S | | |
| r: T321C/C10A/ | | |
| C23S | | |
| Candida | MKIVLVLYDCGKHAADEEKLYGCTENKLGIAN | SEQ ID NO: 192 |
| boidinii formate | WLKDQGHELITTSDKEGGNSVLDQHIPDADII | |
| dehydrogenase | ITTPFHPAYITKERIDKAKKLKLVVVAGVGSD | |
| | HIDLDYINQTGKKISVLEVTGSNVVSVAEHVL | |
| | MTMLVLVRNFVPAHEQIINHDWEVAAIAKDAY | |
| | DIEGKTIATIGAGRIGYRVLERLVPFNPKELL | |
| | YYDYQALPKDAEEKVGARRVENIEELVAQADI | |
| | VTINAPLHAGTKGLINKELLSKFKKGAWLVNT | |
| | ARGAICVAEDVAAALESGQLRGYGGDVWSPQP | |
| | APKDHPWRDMRNKYGAGNAMTPHYSGTTLDAQ | |
| | TRYAEGTKNILESFFTGKFDYRPQDIILLNGE | |
| | YITKAYGKHDKK | |
| Candida | MAHHHHHHKIVLVLYDCGKHAADEEKLYGCTE | SEQ ID NO: 193 |
| boidinii formate | NKLGIANWLKDQGHELITTSDKEGGNSVLDQH | |
| dehydrogenase | IPDADIIITTPFHPAYITKERIDKAKKLKLVV | |
| with N terminal | VAGVGSDHIDLDYINQTGKKISVLEVTGSNVV | |
| His-tag | SVAEHVLMTMLVLVRNFVPAHEQIINHDWEVA | |
| | AIAKDAYDIEGKTIATIGAGRIGYRVLERLVP | |
| | FNPKELLYYDYQALPKDAEEKVGARRVENIEE | |
| | LVAQADIVTINAPLHAGTKGLINKELLSKFKK | |
| | GAWLVNTARGAICVAEDVAAALESGQLRGYGG | |
| | DVWSPQPAPKDHPWRDMRNKYGAGNAMTPHYS | |
| | GTTLDAQTRYAEGTKNILESFFTGKFDYRPQD | |
| | IILLNGEYITKAYGKHDKK | |
| Candida | MKIVLVLYDCGKHAADEEKLYGCTENKLGIAN | SEQ ID NO: 194 |
| boidinii formate | WLKDQGHELITTSDKEGGNSVLDQHIPDADII | |
| dehydrogenase | ITTPFHPAYITKERIDKAKKLKLVVVAGVGSD | |
| with a C | HIDLDYINQTGKKISVLEVTGSNVVSVAEHVL | |
| terminal | MTMLVLVRNFVPAHEQIINHDWEVAAIAKDAY | |
| nanowire (GS) | DIEGKTIATIGAGRIGYRVLERLVPFNPKELL | |
| and C terminal | YYDYQALPKDAEEKVGARRVENIEELVAQADI | |
| MBP (SEQ ID | VTINAPLHAGTKGLINKELLSKFKKGAWLVNT | |
| No. 25). | ARGAICVAEDVAAALESGQLRGYGGDVWSPQP | |
| | APKDHPWRDMRNKYGAGNAMTPHYSGTTLDAQ | |
| | TRYAEGTKNILESFFTGKFDYRPQDIILLNGE | |
| | YITKAYGKHDKKGSHSSYWYAFNNKT | |
| Candida | MHSSYWYAFNNKTGSKIVLVLYDCGKHAADEE | SEQ ID NO: 195 |
| boidinii formate | KLYGCTENKLGIANWLKDQGHELITTSDKEGG | |
| dehydrogenase | NSVLDQHIPDADIIITTPFHPAYITKERIDKA | |
| with a N | KKLKLVVVAGVGSDHIDLDYINQTGKKISVLE | |
| terminal MBP | VTGSNVVSVAEHVLMTMLVLVRNFVPAHEQII | |
| (SEQ ID No. | NHDWEVAAIAKDAYDIEGKTIATIGAGRIGYR | |
| 25), N terminal | VLERLVPFNPKELLYYDYQALPKDAEEKVGAR | |
| nanowire (GS), | RVENIEELVAQADIVTINAPLHAGTKGLINKE | |
| and C terminal | LLSKFKKGAWLVNTARGAICVAEDVAAALESG | |
| His-tag. | QLRGYGGDVWSPQPAPKDHPWRDMRNKYGAGN | |
| | AMTPHYSGTTLDAQTRYAEGTKNILESFFTGK | |
| | FDYRPQDIILLNGEYITKAYGKHDKKLEHHHH | |
| | HH | |
| Candida | MHSSYWYAFNNKTGSKIVLVLYDCGKHAADEE | SEQ ID NO: 196 |
| boidinii formate | KLYGCTENKLGIANWLKDQGHELITTSDKEGG | |
| dehydrogenase | NSVLDQHIPDADIIITTPFHPAYITKERIDKA | |
| with a N | KKLKLVVVAGVGSDHIDLDYINQTGKKISVLE | |
| terminal MBP | VTGSNVVSVAEHVLMTMLVLVRNFVPAHEQII | |
| (SEQ ID No. | NHDWEVAAIAKDAYDIEGKTIATIGAGRIGYR | |
| 25) and N | VLERLVPFNPKELLYYDYQALPKDAEEKVGAR | |
| terminal | RVENIEELVAQADIVTINAPLHAGTKGLINKE | |
| nanowire (GS). | LLSKFKKGAWLVNTARGAICVAEDVAAALESG | |
| | QLRGYGGDVWSPQPAPKDHPWRDMRNKYGAGN | |
| | AMTPHYSGTTLDAQTRYAEGTKNILESFFTGK | |
| | FDYRPQDIILLNGEYITKAYGKHDKKLE | |
| Candida | MAHHHHHHKIVLVLYD(C/A)GKHAADEEKLY | SEQ ID NO: 197 |
| boidinii formate | G(C/S)TENKLGIANWLKDQGHELITTSDKEG | |
| dehydrogenase | GNSVLDQHIPDADIIITTPFHPAYITKERIDK | |
| with a N | AKKLKLVVVAGVGSDHIDLDYINQTGKKISVL | |
| terminal His- | EVTGSNV(V/C)SVAEHVLMTMLVLVRNFVPA | |
| tag and one or | HEQIINHDW(E/C)VAAIAKDAYDIEGKTIAT | |
| more of the | IGAGRIGYRVLERLVPFNPKELLYYDYQALP | |
| following | (K/C)DAEEKVGARRVENIEELVAQADIVTIN | |
| mutations: | APLHAGTKGLIN(K/C)ELLSKFKKGAWLVNT | |
| a: V121C | ARGAICVAEDVAAAL(E/C)(S/C)GQLRGYG | |
| b: E151C | GDVWSPQPAPKDHPWRDMRNKYGAGNAMTPHY | |
| c: K201C | SGTTL(D/C)AQ(T/C)RYAEGTKNILESFFT | |
| d: K241C | GKFDYRPQDIILLNGEYITKAYGKHDKK | |
| e: E272C | | |
| f: S273C | | |
| g: D318C | | |
| h: T321C | | |
| k: V121C/C10A/ | | |
| C23S | | |
| L: E151C/C10A/ | | |
| C23S | | |
| m: K201C/C10A/ | | |
| C23S | | |
| n: K241C/C10A/ | | |
| C23S | | |
| o: E272C/C10A/ | | |
| C23S | | |
| p: S273C/C10A/ | | |
| C23S | | |
| r: T321C/C10A/ | | |
| C23S | | |
| Candida | MKIVLVLYD(C/A)GKHAADEEKLYG(C/S)T | SEQ ID NO: 198 |
| boidinii formate | ENKLGIANWLKDQGHELITTSDKEGGNSVLDQ | |
| dehydrogenase | HIPDADIIITTPFHPAYITKERIDKAKKLKLV | |
| with one or | VVAGVGSDHIDLDYINQTGKKISVLEVTGSNV | |
| more of the | (V/C)SVAEHVLMTMLVLVRNFVPAHEQIINH | |
| following | DW(E/C)VAAIAKDAYDIEGKTIATIGAGRIG | |
| mutations: | YRVLERLVPFNPKELLYYDYQALP(K/C)DAE | |
| a: V121C | EKVGARRVENIEELVAQADIVTINAPLHAGTK | |
| b: E151C | GLIN(K/C)ELLSKFKKGAWLVNTARGAICVA | |
| c: K201C | EDVAAAL(E/C)(S/C)GQLRGYGGDVWSPQP | |
| d: K241C | APKDHPWRDMRNKYGAGNAMTPHYSGTTL | |
| e: E272C | (D/C)AQ(T/C)RYAEGTKNILESFFTGKFDY | |
| f: S273C | RPQDIILLNGEYITKAYGKHDKK | |
| g: D318C | | |
| h: T321C | | |
| k: V121C/C10A/ | | |
| C23S | | |
| L: E151C/C10A/ | | |
| C23S | | |
| m: K201C/C10A/ | | |
| C23S | | |
| n: K241C/C10A/ | | |
| C23S | | |
| o: E272C/C10A/ | | |
| C23S | | |
| p: S273C/C10A/ | | |
| C23S | | |
| r: T321C/C10A/ | | |
| C23S | | |
| Clostridium | MDKKVLTVCPYCGAGCNLYLHVKNGKIIKAEP | SEQ ID NO: 199 |
| ljundahlii, | ANGRTNEGSLCLKGHFGWDFLNDPKILTSRIK | |
| FDH_A | HPMIRKNGELEEVSWDEAISFTASRLSQIKEK | |
| | YGPDSIMGTGCARGSGNEANYIMQKFMRAVIG | |
| | INNVDHCARVCHAPSVAGLAYVLGNGAMSNGI | |
| | HEIDDTDLLLIFGYNGAASHPIVAKRIVRAKQ | |
| | KGAKVIVVDPRITESGRIADLWLPIKNGTNMV | |
| | LVNTFANILINKQFYNKQYVEDHTVGFEEYRS | |
| | IVENYTPEYAEKVTGIPSEDIVEAMKMYSGAK | |
| | NAMILYGMGVCQFAQAVDVVKGLASIALLTGN | |
| | FGRPNVGIGPVRGQNNVQGACDMGALPNVYPG | |
| | YQSVTDDAIREKFEKAWGVKLPNKVGYHLTQV | |
| | PELTLKEDKIKAYYIMGEDPVQSDPDSNEMRE | |
| | TLDKMELVIVQDIFMNKTALHADVILPSTSWG | |
| | EHEGVFSSADRGFQRFRKAVEPKGDVKPDWEI | |
| | ISKIACAMGYNMHYNNTEEIWNELINLCPNFK | |
| | GATYKRLEELGGIQWPCPSENHPGTSYLYKGN | |
| | KFNTPTGKANLFAAEWRPPVEQTDKDYPLVLS | |
| | TVREVGHYSVRTMTGNCRALQQLADEPGYVQV | |
| | NPMDAKAKGIIDGELMRISSRRGSVVARALIT | |
| | ERANKGAVYMTYQWWVGACNELTSNNLDPVSK | |
| | TPELKYCAVKIEAIKDQKEAEKFIKDQYDLLK | |
| | KKMNV | |
| Clostridium | MDKKVLTVCPYCGAGCNLYLHVKNGKIIKAEP | SEQ ID NO: 200 |
| autoethanogenum, | ANGRTNEGSLCLKGHFGWDFLNDPKILTSRIK | |
| FDH_A | HPMIRKNGELEEVSWDEAISFTASRLSQIKEK | |
| | YGPDSIMGTGCARGSGNEANYIMQKFMRAVIG | |
| | TNNVDHCARVCHAPSVAGLAYVLGNGAMSNGI | |
| | HEIDDTDLLLIFGYNGAASHPIVAKRIVRAKQ | |
| | KGAKVIVVDPRITESGRIADLWLPIKNGTNMV | |
| | LVNTFANILINKQFYNKQYVEDHTVGFEEYRS | |
| | IVENYTPEYAEKVTGIPSEDIVEAMKMYSGAK | |
| | NAMILYGMGVCQFAQAVDVVKGLASIALLTGN | |
| | FGRPNVGIGPVRGQNNVQGACDMGALPNVYPG | |
| | YQSVTDDAIRQKFEKAWGVKLPNKVGYHLTQV | |
| | PELTLKEDKIKAYYIMGEDPVQSDPDSNEMRE | |
| | TLDKMELVIVQDIFMNKTALHADVILPSTSWG | |
| | EHEGVFSSADRGFQRFRKAVEPKGDVKPDWEI | |
| | ISKIACAMGYNMHYNNTEEIWNELINLCPNFK | |
| | GATYKRLEELGGIQWPCPSENHPGTSYLYKGN | |
| | KFNTPTGKANLFAAEWRPPVEQTDKDYPLVLS | |
| | TVREVGHYSVRTMTGNCRALQQLADEPGYVQV | |
| | NPMDAKAKGIIDGELMRISSRRGSVVARALIT | |
| | ERANKGAVYMTYQWWVGACNELTSNNLDPVSK | |
| | TPELKYCAVKIEAIKDQKEAEKFIKDQYDLLK | |
| | KKMNV | |
| Clostridium | MDKKVLTVCPYCGAGCNLYLHVKNGKIIKAEP | SEQ ID NO: 201 |
| coskatii, | ANGRTNEGSLCLKGHFGWDFLNDPKILTSRIK | |
| FDH_A | HPMIRKNGELEEVSWDEAISFTASRLSQIKEK | |
| | YGPDSIMGTGCARGSGNEANYIMQKFMRAVIG | |
| | TNNVDHCARVUHAPSVAGLAYVLGNGAMSNGI | |
| | HEIDDTDLLLIFGYNGAASHPIVAKRIVRAKQ | |
| | KGAKVIVVDPRITESGRIADLWLPIKNGTNMV | |
| | LVNTFANILINKQFYNKQYVEDHTVGFEEYRS | |
| | IVENYTPEYAEKVTGIPSEDIVEAMKMYSGAK | |
| | NAMILYGMGVCQFAQAVDVVKGLASIALLTGN | |
| | FGRPNVGIGPVRGQNNVQGACDMGALPNVYPG | |
| | YQSVTDDAIREKFEKAWGVKLPNKIGYHLTQV | |
| | PELTLKEDKIKAYYIMGEDPVQSDPDSNEMRE | |
| | TLDKMELVIVQDIFMNKTALHADVILPSTSWG | |
| | EHEGVFSSADRGFQRFRKAVEPKGDVKPDWEI | |
| | ISEIACAMGYNMHYNNTEEIWNELINLCPNFK | |
| | GATYKRLEELGGIQWPCPSENHPGTSYLYKGN | |
| | KFNTPTGKANLFAAEWRPPVEQTDKDYPLVLS | |
| | TVREVGHYSVRTMTGNCRALQQLADEPGYVQI | |
| | NPMDAKAEGIIDGELMRISSRRGSVVARALVT | |
| | ERANKGAVYMTYQWWVGACNELTSNNLDPVSK | |
| | TPELKYCAVKIEAIKDQKEAEKFIKDQYDLLK | |
| | KKMNV | |
| Clostridium | MDKKVLTVCPYCGAGCKLYLHVKDGKIIKAEP | SEQ ID NO: 202 |
| ragsdalei, | ANGRTNEGSLCLKGRFGWDFLNDPKILTSRIK | |
| FDH_A | HPMIRKNGELEEVSWDEAISFTASKLSQIKEK | |
| | YGPDSIMGTGCARGSGNEANYVMQKFMRAVIG | |
| | TNNVDHCARVUHAPSVAGLAYVLGNGAMSNGI | |
| | HEIDDTDLLLIFGYNGAASHPIVAKRIVRAKQ | |
| | KGAKVIVVDPRITESGRIADLWLPIKNGTNMV | |
| | LVNTFANILINKQFYDKQYVEDHTVGFEEYKS | |
| | IVEDYTPEYAEKVTGIPAEDIVEAMKMYSSAK | |
| | NAMILYGMGVCQFAQAVDVVKGLASIALLTGN | |
| | FGRPNVGIGPVRGQNNVQGACDMGALPNVYPG | |
| | YQSVTDDAIREKFEKAWGVKLSNKVGYHLTRV | |
| | PELTLKEDKIKAYYIMGEDPAQSDPDSNEMRE | |
| | TLDKMELVIVQDIFMNKTALHADVILPSTSWG | |
| | EHEGVFSSADRGFQRFRKAVEPKGDVKPDWEI | |
| | ISEIACAMGYDMHYNNTEEIWDELINLCPNFK | |
| | GATYKRLDELGGIQWPCPSEDHPGTSYLYKGN | |
| | KFNTPTGKANLFAAEWRPPIEKTDEEYPLVLS | |
| | TVREVGHYSVRTMTGNCRALQQLADEPGYVQI | |
| | NPVDAKAKKIIDGELMRVSSRRGSVVARALVT | |
| | ERANKGAVYMTYQWWVGACNELTANNLDPVSK | |
| | TPELKYCAVKVEAIEDQKEAEKFIKDQYASIK | |
| | KKMNV | |
| Paraclostridium | MEKKVLTVCPYCGAGCQLYLVVKDNEIVRAEP | SEQ ID NO: 203 |
| bifermentans, | ANGRTNEGNLCLKGYYGWDFLNDPKILTSRLK | |
| FDH_A | KPMIRKNGKLEEVEWKEAIDYTASRLNEIREK | |
| | YGPDAIMGTGSARGPGNEANYVMQKFMRAAIG | |
| | TNNIDHCARVCHAPSVAGLAYSLGNGAMSNSI | |
| | PEIENSDLLFIFGYNGADSHPIVARRIIRAKE | |
| | KGAKLIVTDPRVTESVRISDMWLPIKGGTNMI | |
| | LVNAFANVLIEENLYNKEYVEKYTEGFEEYKE | |
| | MVKKYTPEYAEKMTNVPAEDIRKAMREYANAK | |
| | NATILYGMGVCQFGQAVDVVKGLASLALLTGN | |
| | FGRESVGIGPVRGQNNVQGTCDMGTLPNLFPG | |
| | YQKVTDDKVRKKFEKAWGVKLSSKPGITLTEV | |
| | PHLTLKDNKVKAYYIFGEDPVQSDPHASEVRE | |
| | TLDAMEFVVVQDIFMNKTALHADVVLPATSWG | |
| | EHDGVYSSADRGFQRMRKAVEPKGDVKPDWQI | |
| | MCEISTAMGYPMSYNNTKEIWDEMISLSPLFA | |
| | GASYEKIEKQGSVLWPCTDESHKGTPYLYEGN | |
| | KFETESGKGKLFACEWRPPKEIPDEEYPLVLC | |
| | TVREVGHYSVRTMTGNCRALRALEDEPGYIQL | |
| | SIEDASKLGIKDKELVRVSSRRGSILTRALVT | |
| | DRVIKGATYMTYSWWIGSCNELTIDNLDPISK | |
| | TPEYKYCAIKVISIKDQDSAEQYIKDEYEKIR | |
| | KQMLIEK | |
| Candida | MKIVLVLYDCGKHAADEEKLYGCTENKLGIAN | SEQ ID NO. 204 |
| boidinii formate | WLKDQGHELITTSDKEGGNSVLDQHIPDADII | |
| dehydrogenase | ITTPFHPAYITKERIDKAKKLKLVVVAGVGSD | |
| with a E272C | HIDLDYINQTGKKISVLEVTGSNVVSVAEHVL | |
| mutation | MTMLVLVRNFVPAHEQIINHDWEVAAIAKDAY | |
| | DIEGKTIATIGAGRIGYRVLERLVPFNPKELL | |
| | YYDYQALPKDAEEKVGARRVENIEELVAQADI | |
| | VTINAPLHAGTKGLINKCLLSKFKKGAWLVNT | |
| | ARGAICVAEDVAAALCSGQLRGYGGDVWSPQP | |
| | APKDHPWRDMRNKYGAGNAMTPHYSGTTLDAQ | |
| | TRYAEGTKNILESFFTGKFDYRPQDIILLNGE | |
| | YITKAYGKHDKK | |
| Candida | MKIVLVLYDCGKHAADEEKLYGCTENKLGIAN | SEQ ID NO: 205 |
| boidinii formate | WLKDQGHELITTSDKEGGNSVLDQHIPDADII | |
| dehydrogenase | ITTPFHPAYITKERIDKAKKLKLVVVAGVGSD | |
| with a C | HIDLDYINQTGKKISVLEVTGSNVVSVAEHVL | |
| terminal His- | MTMLVLVRNFVPAHEQIINHDWEVAAIAKDAY | |
| tag and a | DIEGKTIATIGAGRIGYRVLERLVPFNPKELL | |
| E272C | YYDYQALPKDAEEKVGARRVENIEELVAQADI | |
| mutation | VTINAPLHAGTKGLINKCLLSKFKKGAWLVNT | |
| (enzyme e) | ARGAICVAEDVAAALCSGQLRGYGGDVWSPQP | |
| | APKDHPWRDMRNKYGAGNAMTPHYSGTTLDAQ | |
| | TRYAEGTKNILESFFTGKFDYRPQDIILLNGE | |
| | YITKAYGKHDKKHHHHHH | |

In embodiments, the enzyme is:
(i) wild type formate dehydrogenase from Candida boidinii
(ii) formate dehydrogenase from Candida boidinii comprising one or more surface cysteine mutations, e.g.: D9C, S45C, V121C, E151C, R174C, K201C, K241C, K249C, A269C, E272C, S273C, L317C, D318C, T321C, and/or K328C; and optionally wherein the enzyme has a His Tag. In a specific embodiment the mutation is E272C.

In another embodiment, the formate hydrogenase may include a his-tag or other nanowires or MBPs. In one Example, the nanowire may include the amino acid residues GS. In another embodiment, the MBP may be SEQ ID NO. 25. In another embodiment, the formate dehydrogenase from Candida boidinii has an amino acid sequence from SEQ ID NO. 190.

In another embodiment, the formate dehydrogenase from Candida boidinii can include a surface cysteine and/or stabilizing cysteine amino acid mutation, e.g., A10C and/or one or more of D9C, S45C, V121C, E151C, R174C, K201C, K241C, K249C, A269C, E272C, S273C, L317C, D318C, T321C, or K328C; and optionally wherein the enzyme has a His Tag. In another embodiment, the formate dehydrogenase from Candida boidinii has an amino acid sequence from SEQ ID NO. 110, SEQ ID NO. 111, SEQ ID NO. 112, or SEQ ID NO. 191.

embodiments, the enzyme is formate dehydrogenase from Candida boidinii i comprising one or more surface cysteine mutations, e.g.: D9C, S45C, V121C, E151C, R174C, K201C, K241C, K249C, A269C, E272C, S273C, L317C, D318C, T321C, or K328C wherein the enzyme optionally comprises a His Tag. In embodiments, the enzyme comprises a D9C mutation. In embodiments, the enzyme comprises a S45C mutation. In embodiments, the enzyme comprises a V121C mutation. In embodiments, the enzyme comprises a E151C mutation. In embodiments, the enzyme comprises a R174C mutation. In embodiments, the enzyme comprises a K201C mutation. In embodiments, the enzyme comprises a K241C mutation. In embodiments, the enzyme comprises a K249C mutation. In embodiments, the enzyme comprises a A269C mutation. In embodiments, the enzyme comprises a E272C mutation. In embodiments, the enzyme comprises a S273C mutation. In embodiments, the enzyme comprises a L317C mutation. In embodiments, the enzyme comprises a D318C mutation. In embodiments, the enzyme comprises a T321C mutation. In embodiments, the enzyme comprises a K328C mutation. In embodiments, the enzyme comprises a His-tag. In embodiments the enzyme does not comprise a His-tag.

In embodiments, the enzyme is formate dehydrogenase from Candida boidiniicomprising a surface cysteine and stabilizing A10C mutation, e.g., A10C and one or more of D9C, S45C, V121C, E151C, R174C, K201C, K241C, K249C, A269C, E272C, S273C, L317C, D318C, T321C, or K328C; and optionally wherein the enzyme comprises a His Tag. In embodiments, the enzyme comprises a D9C mutation and a A10C mutation. In embodiments, the enzyme comprises a S45C mutation and a A10C mutation. In embodiments, the enzyme comprises a V121C mutation and a A10C mutation. In embodiments, the enzyme comprises a E151C mutation and a A10C mutation. In embodiments, the enzyme comprises a R174C mutation and a A10C mutation. In embodiments, the enzyme comprises a K201C mutation and a A10C mutation. In embodiments, the enzyme comprises a K241C mutation and a A10C mutation. In embodiments, the enzyme comprises a K249C mutation and a A10C mutation. In embodiments, the enzyme comprises a A269C mutation and a A10C mutation. In embodiments, the enzyme comprises a E272C mutation and a A10C mutation. In embodiments, the enzyme comprises a S273C mutation and a A10C mutation. In embodiments, the enzyme comprises a L317C mutation and a A10C mutation. In embodiments, the enzyme comprises a D318C mutation and a A10C mutation. In embodiments, the enzyme comprises a T321C mutation and a A10C mutation. In embodiments, the enzyme comprises a K328C mutation and a A10C mutation. In embodiments, the enzyme comprises a His-tag. In embodiments the enzyme does not comprise a His-tag.

In another embodiment, the Candida boidinii formate dehydrogenase has a single point mutation at residue 151. In another specific embodiment, the mutation is E151C.

In another specific embodiment, the Candida boidinii formate dehydrogenase has a single point mutation at one or more of the following residues: 121, 151, 201, 241, 272, 273, 318, 321, 10, and/or 23. In a specific embodiment, the one or more of the following residues are mutated as follows:
a: V121C
b: E151C
c: K201C
d: K241C
e: E272C
f: S273C
g: D318C
h: T321C
k: V121C/C10A/C23S
L: E151C/C10A/C23S
m: K201C/C10A/C23S
n: K241C/C10A/C23S
o: E272C/C10A/C23S
p: S273C/C10A/C23S
r: T321C/C10A/C23S

In embodiments, the enzyme is wild type formate dehydrogenase from Myceliophthora thermophila optionally comprising a His-Tag. In embodiments, the wild type Formate dehydrogenase is from Myceliophthora thermophila comprising a His-tag. In embodiments the enzyme does not comprise a His-tag. In a specific embodiment, the Formate dehydrogenase enzyme comprises the amino acid sequence from SEQ ID NO: 115 or SEQ ID NO: 116.

In embodiments, the enzyme is wild type Formate dehydrogenase from Rhodobacter capsulatus optionally comprising a His-Tag. In embodiments, the enzyme comprises a His-tag. In embodiments the enzyme does not comprise a His-tag.

In embodiments, the enzyme is wild type Formate dehydrogenase from Rhodobacter capsulatus wherein the enzyme is characterized by the removal of one or more cysteines: fdsA-C121G, fdsA-C124G; and optionally wherein the enzyme comprises a His-Tag. In embodiments, the enzyme comprises a His-tag. In embodiments the enzyme does not comprise a His-tag.

In embodiments, the enzyme is wild type or mutant Formate dehydrogenase from Rhodobacter capsulatus. In a specific embodiment, the wild type or mutant Formate dehydrogenase may comprise 1, 2, 3 or 4 of sequences from fdsGBAD, i.e., one of the domains from the heterotetramer of Formate dehydrogenase from Rhodobacter capsulatus, In another embodiment, the Formate dehydrogenase from Rhodobacter capsulatus is a tetramer that dimerizes to yield a dimer of heterotetramers, i.e., 2×fdsGBAD 1, 2, 3 or 4 of sequences from fdsGBAD wherein 1, 2, 3, or 4 sequences from each tetramer, may be different than their corresponding sequence in the dimer. In another embodiment, the Formate dehydrogenase from Rhodobacter may comprise the addition of one or more cysteines: fdsG-A89C, fdsA-D120C, fdsA-E143C in one or two of the tetramers from the dimer; and optionally wherein the enzyme comprises a His-Tag. In embodiments, the enzyme comprises a His-tag. In embodiments the enzyme does not comprise a His-tag.

In embodiments, the enzyme is formate dehydrogenase from Rhodobacter capsulatus with a His-tag (e.g, as disclosed herein) with one or more cysteines added e.g., fdsG-A89C, fdsA-D120C, and/or fdsA-E143C. In embodiments the formate dehydrogenase from Rhodobacter capsulatus with a His-tag comprises a fdsG-A89C mutation. In embodiments the formate dehydrogenase from Rhodobacter capsulatus with a His-tag comprises a fdsA-D120C mutation. In embodiments the formate dehydrogenase from Rhodobacter capsulatus with a His-tag comprises a fdsA-E143C mutation. In another embodiment, the mutations may be in one or two of the tetramers from the dimer.

In embodiments, the enzyme is formate dehydrogenase from Rhodobacter capsulatus with a His-tag (e.g, as disclosed herein) with one or more cysteines removed e.g., fdsA-C121G and/or fdsA-C124G. In embodiments the formate dehydrogenase from Rhodobacter capsulatus with a His-tag comprises a fdsA-C121G mutation. In embodiments the formate dehydrogenase from Rhodobacter capsulatus with a His-tag comprises a fdsA-C124G mutation. In another embodiment, the mutations may be in one or two of the tetramers from the dimer.

In embodiments, the enzyme is formate dehydrogenase from Rhodobacter capsulatus without a His-tag (e.g, as disclosed herein) with one or more cysteines added e.g., fdsG-A89C, fdsA-D120C, and/or fdsA-E143C. In embodiments the formate dehydrogenase from Rhodobacter capsulatus without a His-tag comprises a fdsG-A89C mutation. In embodiments the formate dehydrogenase from Rhodobacter capsulatus without a His-tag comprises a fdsA-D120C mutation. In embodiments the formate dehydrogenase from Rhodobacter capsulatus without a His-tag comprises a fdsA-E143C mutation. In another embodiment, the mutations may be in one or two of the tetramers from the dimer.

In embodiments, the enzyme is formate dehydrogenase from Rhodobacter capsulatus without a His-tag (e.g, as disclosed herein) with one or more cysteines removed e.g., fdsA-C121G and/or fdsA-C124G. In embodiments the formate dehydrogenase from Rhodobacter capsulatus without a His-tag comprises a fdsA-C121G mutation. In embodiments the formate dehydrogenase from Rhodobacter capsulatus without a His-tag comprises a fdsA-C124G mutation. In another embodiment, the mutations may be in one or two of the tetramers from the dimer. In another embodiment, the formate dehydrogenase from Rhodobacter capsulatus has an amino acid sequence or may comprise one or more sequences from SEQ ID NO. 117-SEQ ID NO. 119 or SEQ ID NO. 121-SEQ ID NO. 123. In another embodiment, the formate dehydrogenase from Rhodobacter capsulatus may utilize or comprise a chaperone sequence, such as from SEQ ID NO. 120.

In another embodiment, the formate producing enzyme may be from Clostridium ljundahlii, FDH_A, Clostridium autoethanogenum, FDH_A, Clostridium coskatii, FDH_A, Clostridium ragsdalei, FDH_A, Paraclostridium bifermentans, FDH_A, Desulfovibrio vulgaris Hildenborough FDH-AB, Escherichia coli FDH-H, Desulfovibrio desulfuricans FDH_ABC, Cupriavidus necator FDH-DABG, Rhodobacter aestuarii FDH-ABG, Candida methylica formate dehydrogenase, Chaetomium thermophulum formate dehydrogenase, Kwoniella shandongensis formate dehydrogenase, Fonsecaea multimorphosa CBS 102226 formate dehydrogenase, Sclerotinia sclerotiorum 1980 UF-70 formate dehydrogenase, and/or Syntrophobacter fumaroxidans FDH1.

In a specific embodiment the Enzyme includes an amino acid sequence from SEQ ID NO. 110-123 or 190-205.

In another embodiment, the present invention includes a peptide or an amino acid sequence of about 75% to about 99.9% identical to any sequence from SEQ ID NO. 110-123. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% any sequence from SEQ ID NO. 110-123.

In another embodiment, the present invention includes a peptide or an amino acid sequence of about 75% to about 99.9% identical to any sequence from SEQ ID NO. 189-205. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% any sequence from SEQ ID NO. 189-205.

In another embodiment, the enzyme and/or oxidoreductase enzyme are enzymes and/or oxidoreductase enzymes as in Examples 1-16.

Embodiments of the present invention also include methods for producing formate, formic acid, or a salt thereof by adding a starting agent or starting substrate to an enzymatic reactor cell described herein. In another embodiment, the enzyme of the enzymatic reactor cell comprises formate dehydrogenase. In another embodiment, the starting agent or stating substrate is selected from one or more of the group consisting of aqueous carbon dioxide, gaseous carbon dioxide, bicarbonate, carbonate, carbonic acid,, water, and/or a salt thereof. In another embodiment, the methods include steps or methods as described in Examples 1-16 described herein. In a specific embodiment, the formic acid or formate is produced in a reactor cell described herein in a temperature range between about 20-60° C.; pH range of about 3.0-7.5; a current density of about: 0.005 to 200 mA/cm2 with a cathodic potential above −3V vs NHE.

### EXAMPLES

### Example 1: Enzymatic Reactor Cell Including Formate Dehydrogenase on Carbon Surface

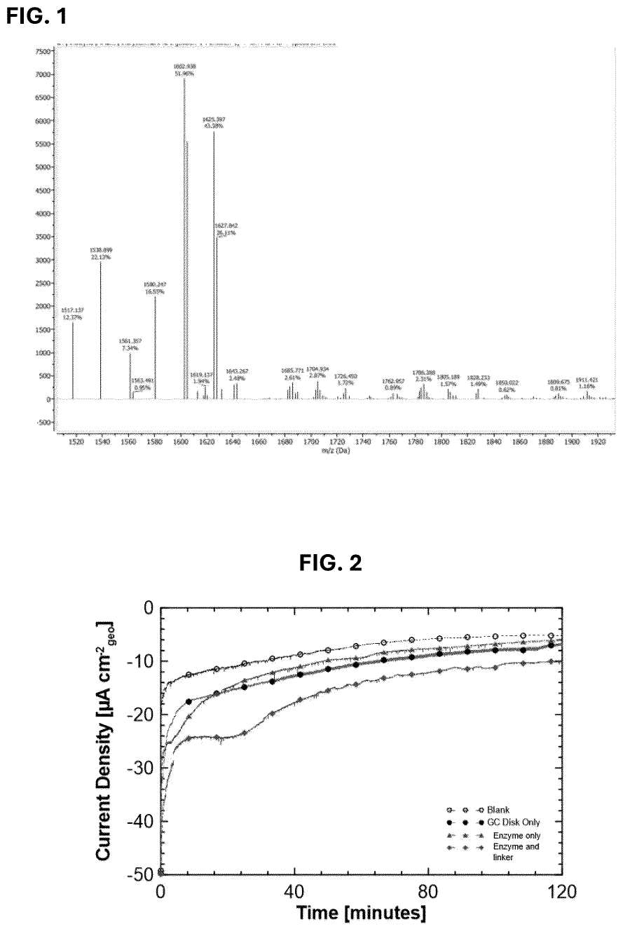

The assembly of an enzymatic reactor cell utilising a reactor cell with a coated carbon surface is described below. The linker has been prepared by adding a graphite MBP (SEQ ID NO: 25) to a glutaraldehyde nanowire used for chemical linkage. Specifically, the graphite peptide and glutaraldehyde were added together and the peptide-nanowire conjugate samples were desalted using a C18 Resin “ZipTip” following manufacturer directions. The resultant peptide solutions were evaporated on a plate using a sinapinic acid matrix. Spectra were collected on a Bruker microflex LT MALDI Biotyper mass spectrometer and analysis completed using MNova (MestreLab) software. FIG. 1 shows the successful conjugation of glutaraldehyde to SEQ ID NO. 25.

Masses for unmodified peptide and the peptide conjugate are both observed (+84 Da) at 1517.050 Da and 1602.9 Da, respectively. The peptide conjugate was combined to the formate dehydrogenase by methods as described herein with glutaraldehyde linker chemistry, and subsequently the full conjugated enzyme-linker was adsorbed to the carbon surface. FIG. 2 shows a current density of blank (substrate free with the enzyme linker construct); glassy carbon disk only; enzyme only; and enzyme linker construct).

Activity of the enzyme on the carbon disks was also confirmed by testing for formate. Formate can be detected by monitoring the 1:1 conversion of formate to CO₂ by formate dehydrogenase which simultaneously uses NAD+ to produce NADH. By measuring the A340, which is a direct measure of NADH concentration, the concentration of formate that was consumed can be calculated.

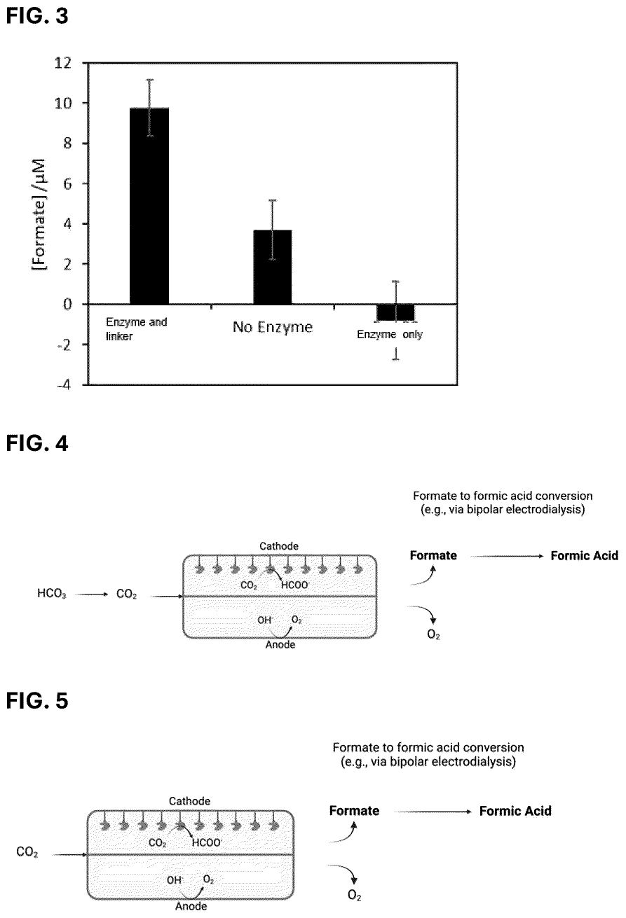

Specifically, formate standards were prepared in 100 mM sodium bicarbonate, 100 mM Tris at 0.2, 0.1, 0.02, 0.01, and 0 mM. 200 mM Tris-HCl, pH 7.4 was prepared as an operating buffer. A 10 mg/mL stock of just formate dehydrogenase enzyme as a control was also prepared. A 3.6 mM NAD+, 164 mM Tris solution was prepared. 40 μL of analyte (formate standard or sample from 2 hour chronoamperometry), 5 μL of formate dehydrogenase, and 55 μL of the NAD+/Tris solution were combined and then the absorbance at 340 nm to measure the NADH formation. FIG. 3 shows the concentration of formate detected after 2 hour chronoamperometry reactions for enzyme-linker (n=3) and no enzyme (glassy carbon electrode alone) (n=3), and just enzyme with no linker (n=2). The concentration of formate was determined by the standard colorimetric assay (using formate dehydrogenase to consume formate and produce UV active NADH).

### Example 2: Producing Formate from Bicarbonate

The following example utilizes formate dehydrogenase to produce formate in an enzymatic reactor cell (FIG. 4). In this example, formate dehydrogenase is immobilized on the cathode surface by the use of a linker that specifically binds to the cathode surface by a surface binding moiety. Bicarbonate is added to the reactor cell where it acts as a source of CO₂. Formate dehydrogenase utilizes the CO₂ to produce formate.

When suitable counter ions are present in solution, the produced formate may be isolated as a salt. For example, sodium formate or potassium formate. The isolated salt may be further converted to formic acid by standard techniques such as bipolar electrodialysis.

The formate is produced in the reactor cells under the following conditions: temperature range: 20-60° C.; pH range: 3.5-9; current density: 0.005 to 200 mA/cm² with a cathodic potential above −3V vs NHE. The reactor cell provides a purity of 5-99 percent and a yield of 5-99 percent.

### Example 3: Producing Formate from CO₂

The following example utilizes formate dehydrogenase to produce formate in an enzymatic reactor cell (FIG. 5). In this example, formate dehydrogenase is immobilized on the cathode surface by the use of a linker that specifically binds to the cathode surface by a surface binding moiety. CO₂ is added to the reactor cell where formate dehydrogenase utilizes the CO₂ to produce formate.

When suitable counter ions are present in solution, the produced formate may be isolated as a salt. For example, sodium formate or potassium formate. The isolated salt may be further converted to formic acid by standard techniques such as bipolar electrodialysis.

The formate is produced in the reactor cells under the following conditions: temperature range: 20-60° C.; pH range: 3.5-9; current density: 0.005 to 200 mA/cm² with a cathodic potential above −3V vs NHE. The reactor cell provides a purity of 5-99 percent and a yield of 5-99 percent.

### Example 4: Producing Formic Acid from Bicarbonate

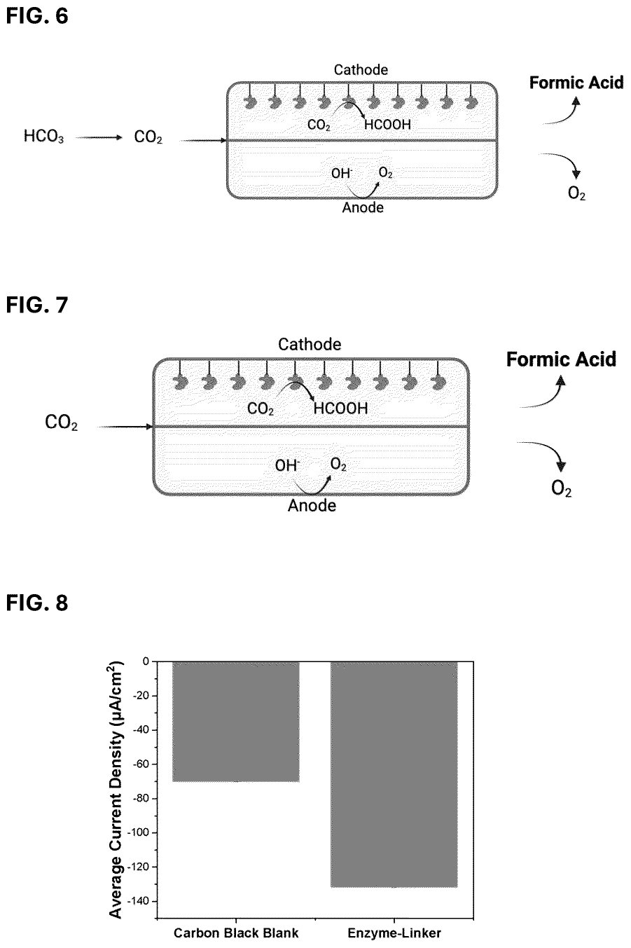

The following example utilizes formate dehydrogenase to produce formic acid in an enzymatic reactor cell (FIG. 6). In this example, formate dehydrogenase is immobilized on the cathode surface by the use of a linker that specifically binds to the cathode surface by a surface binding moiety. Bicarbonate is added to the reactor cell where it acts as a source of CO₂. At appropriate pH, formate dehydrogenase utilizes the CO₂ to produce formic acid.

The formic acid is produced in the reactor cells under the following conditions: temperature range: 20-60° C.; pH range: 3.0-9; current density: 0.005 to 200 mA/cm² with a cathodic potential above −3V vs NHE. The reactor cell provides a purity of 5-99 percent and a yield of 5-99 percent.

### Example 5: Producing Formic Acid from CO₂

The following example utilizes formate dehydrogenase to produce formic acid in an enzymatic reactor cell (FIG. 7). In this example, formate dehydrogenase is immobilized on the cathode surface by the use of a linker that specifically binds to the cathode surface by a surface binding moiety. CO₂ is added to the reactor cell where, at appropriate pH, formate dehydrogenase utilizes the CO₂ to produce formic acid.

The formic acid is produced in the reactor cells under the following conditions: temperature range: 20-60° C.; pH range: 3.0-9; current density: 0.005 to 200 mA/cm² with a cathodic potential above −3V vs NHE. The reactor cell provides a purity of 5-99 percent and a yield of 5-99 percent.

### Example 6: Enzymatic Reactor Cell Containing Formate Dehydrogenase on Graphite Foil

The following example utilizes formate dehydrogenase to produce formic acid or formate from CO₂ or bicarbonate in an enzymatic reactor cell. In this example, formate dehydrogenase is immobilized on a carbon electrode by use of a linker that specifically binds to carbon surfaces through a surface binding moiety. Specifically, formate dehydrogenase is bound to a graphite foil electrode.

The formate is produced in the reactor cells under the following conditions: temperature range: 20-60° C.; pH range: 3.5-79; current density: 0.005 to 200 mA/cm² with a cathodic potential above −3V vs NHE. The reactor cell provides a purity of 5-99 percent and a yield of 5-99 percent.

### Example 7: Enzymatic Reactor Cell Containing Formate Dehydrogenase on Carbon Paper

The following example utilizes formate dehydrogenase to produce formic acid or formate from CO₂ or bicarbonate in an enzymatic reactor cell. In this example, formate dehydrogenase is immobilized on a carbon electrode by use of a linker that specifically binds to carbon surfaces through a surface binding moiety. Specifically, formate dehydrogenase is bound to a carbon paper electrode.

The formate is produced in the reactor cells under the following conditions: temperature range: 20-60° C.; pH range: 3.5-9; current density: 0.005 to 200 mA/cm2 with a cathodic potential above −3V vs NHE. The reactor cell provides a purity of 5-99 percent and a yield of 5-99 percent.

### Example 8: Formate/Formic Acid Quantification

To test for the production of formate and/or formic acid in the reactor cell, a portion of the catholyte was adjusted to pH 7.4 alongside a series of formate standards. These samples/standards were combined with NAD+ (final concentration=2 mM) and treated with wt formate dehydrogenase from Candida boidinii (CbFDHWT). Formate dependent NAD+ consumption was detected by monitoring absorbance of NADH formed. Samples were reacted for 90 min and the absorbance at 340 nm recorded. Standards were used to prepare a calibration curve and this curve used to determine the concentration(s) of formate in the analyte solutions.

### Example 9: Assessing the Stability of a Formate Dehydrogenase Containing 3-Electrode Reactor Cell

To test for the production of formic acid in the reactor cell, a portion of the catholyte was adjusted to pH 7.4 alongside a series of formate standards. These samples/standards were combined with NAD+ (final concentration=2 mM) and treated with wt formate dehydrogenase from Candida boidinii (CbFDHWT). Formate dependent NAD+ consumption was detected by monitoring absorbance of NADH formed. Samples were reacted for 90 min and the absorbance at 340 nm recorded. Standards were used to prepare a calibration curve and this curve used to determine the concentration(s) of formate in the analyte solutions.

### Example 10: Assessing the Stability of a Formate Dehydrogenase Containing Flow Cell

Submerge an appropriate cathode (e.g., 10 cm2 carbon paper) in sufficient volume (e.g., 1.5 mL) of biomaterial (such as enzyme and a linker with a material binding peptide). After 10 minutes, submerge the cathode in buffer for ten minutes twice to remove excess biomaterial.

Place the cathode in an electrolyser flow cell, circulate an appropriate electrolyte (e.g., 0.102 M sodium phosphate dibasic, 0.05 M citric acid, 25 mM NaHCO₃, pH 5.5-6). If gaseous CO₂ is the reagent, supply the flow cell with gaseous CO₂. If bicarbonate is the source of CO₂, ensure the electrolyte contains bicarbonate.

To test the stability of formate dehydrogenase, a chronoamperometry run is carried out. A potential of −1.0 V vs NHE or −1.2 V vs NHE or −1.4 V vs NHE is applied for 20 minutes or longer as needed.

### Example 11: Electrical Impedance Spectroscopy

This study determines the resistance intrinsic to the reactor cell.

To a working electrode (e.g., 0.84 cm² rectangle of graphite foil such that the total surface area is 1.68 cm²) deposit a volume of biomaterial (such as enzyme and a linker with a material binding peptide) such that the electrode surface is completely submerged (e.g., 200 μL). After 10 minutes, rinse the excess biomaterial from the surface by submerging the electrode in buffer.

To an appropriate container (3-electrode flask, crucible, etc.), the platinum counter electrode (e.g., mesh, wire, flag, etc.), saturated Ag/AgCl reference electrode, prepared working electrode, and electrolyte (e.g., 0.102 M sodium phosphate dibasic, 0.05 M citric acid, 25 mM NaHCO3, pH 5.5-6) are added.

The electrical impedance is then run with a fixed current or voltage, applying a perturbation, and scan across a range of frequencies.

The obtained R_{CT}<10,000Ω.

### Example 12: Producing Formate with an Enzyme-Linker Absorbed to Carbon Black

Enzyme production: The enzyme (Candida boidinii formate dehydrogenase) was expressed recombinantly in E. coli using standard techniques. A plasmid was prepared encoding the enzyme gene, amino acid linker sequence (nanowire GS amino acid sequence), and material binding peptide (MBP) SEQ ID No. 25. The material binding peptide, nanowire, and enzyme are expressed as a continuous construct. The material binding peptide and nanowire are at the C terminus, which was verified by sequencing. The verified plasmid was transformed into an expression strain of E. coli. Following initial growth of the E. coli, induction was performed and the cultures were permitted to grow for 16-20 hours for recombinant expression. Purified protein was obtained by cell lysis (tip sonication) followed by purification by immobilised metal affinity chromatrography. Identity of the protein was confirmed by SDS-PAGE gel.

### Bio-Electrochemical Formate Production:

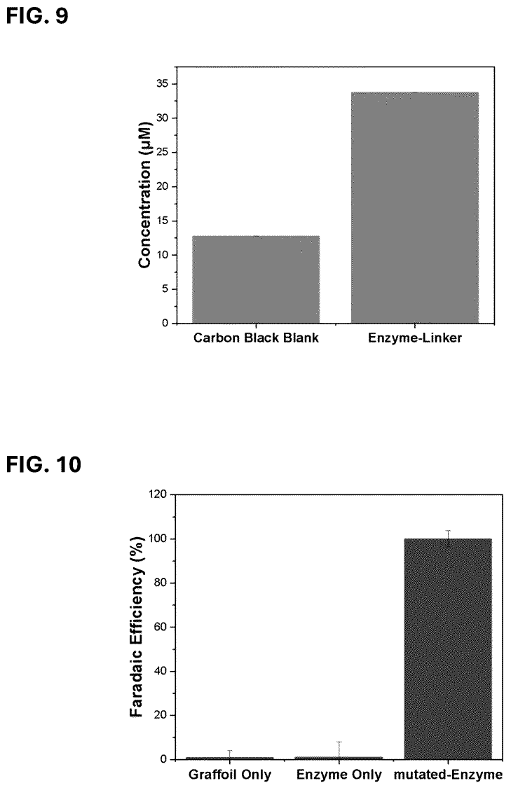

A screen-printed electrode (SPE) comprised a carbon black working electrode, a carbon black counter electrode, and an Ag/AgCl reference electrode was used. It was placed in a petri dish, and sufficient volume of enzyme-linker solution was deposited onto the working electrode to coat the surface. After a 10-60 minute incubation, unabsorbed enzyme-linker was removed. The electrode was then washed with KPi for 10 minutes. The SPE was mounted onto a holder, and 500 μL of electrolyte (90 mM sodium acetate, 25 mM sodium bicarbonate) was added. Chronoamperometry was performed at −1 V (vs NHE) for 60 minutes. FIGS. 8 and 9 present the average current density and formate production of the enzyme-linker. A control experiment, conducted identically with an SPE lacking the enzyme-linker, showed lower current density and formate concentration, confirming the enzyme-linker's catalytic activity.

Product Assay: Formate concentrations in electrolyte were determined by an enzymatic assay. Samples containing formate were combined with NAD+ and a formate dehydrogenase. These were allowed to react under ambient conditions.As formate is consumed a stoichiometric amount of NAD+ is converted to NADH. After reaction completion the concentration of NADH was determined by measuring absorbance at 340 nm and calibration against standards prepared in the same electrolyte matrix as the samples. Each sample and calibration standard was prepared in triplicate and errors reported are the standard deviation from the mean for these replicates.

### Example 13: Producing Formate with a Mutant Enzyme Absorbed to Graphene Foil

Enzyme production: The enzyme (Candida boidinii formate dehydrogenase) was expressed recombinantly in E. coli using standard techniques. A plasmid was prepared encoding the enzyme gene with a surface cystine mutation. The verified plasmid was transformed into an expression strain of E. coli. Following the initial growth of the E. coli, over expression was induced and expression allowed to proceed for 16-20h.Purified protein was obtained by cell lysis (tip sonication) followed by purification by immobilized metal affinity chromatography. Identity of the protein was confirmed by SDS-PAGE gel.

### Bio-Electrochemical Formate Production:

Graphene foil (hereafter referred to as “graffoil”) was cut into rectangular pieces measuring 1 cm×3 cm. The electrode was cleaned by sequentially soaking it in isopropyl alcohol and Milli-Q water, followed by drying with argon (Ar) gas. The central region of the graffoil was masked with Kapton tape, leaving two exposed areas: one measuring 0.5 cm×1 cm for electrochemical measurements and another measuring 0.5 cm×0.5 cm for electrical connections. The prepared graffoil was placed in a small weigh boat, and a volume of enzyme sufficient to coat the exposed electrode surface was deposited. The electrode was incubated for 10-60 minutes, after which excess enzyme was removed by rinsing with KPi. The H-cell was assembled using a platinum counter electrode, an Ag/AgCl reference electrode, and a Nafion membrane as the separator. The working electrode compartment was purged with Ar gas for 30 minutes prior to electrochemical characterization. Chronoamperometry was performed at −1 V (vs. NHE) for 2 hours. Control experiments were conducted using graffoil alone and enzyme alone for comparison.

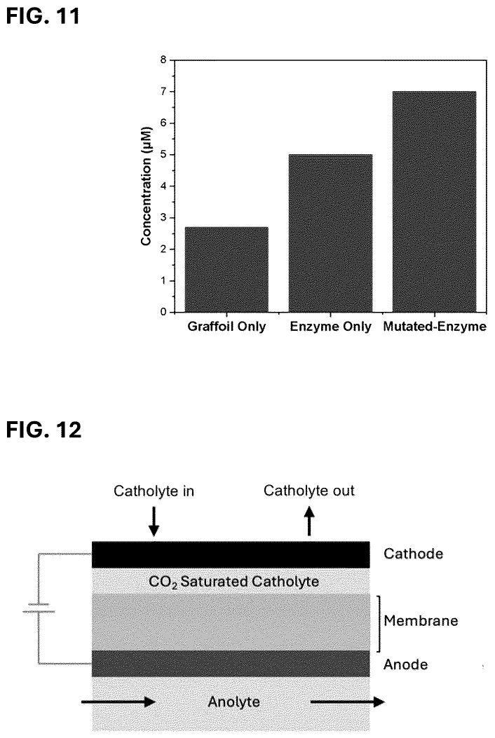

Product Assay (FIGS. 10 and 11): Formate concentrations in electrolyte were determined by an enzymatic assay. Samples containing formate were combined with NAD+ and a formate dehydrogenase. These were allowed to react under ambient conditions. As formate is consumed a stoichiometric amount of NAD+ is converted to NADH.

This data demonstrates the superior performance of just the mutant enzyme provided herein. After reaction completion the concentration of NADH was determined by measuring absorbance at 340 nm and calibration against standards prepared in the same electrolyte matrix as the samples. Each sample and calibration standard was prepared in triplicate and errors reported are the standard deviation from the mean for these replicates.

### Example 14: Producing Formate with an Enzyme-Linker Absorbed to a Carbon Felt Surface

Enzyme production: The enzyme (Candida boidinii formate dehydrogenase) was expressed recombinantly in E. coli using standard techniques. A plasmid was prepared encoding the enzyme gene, amino acid linker sequence (nanowire GS amino acid sequence), and material binding peptide (MBP) SEQ ID No. 25. The material binding peptide, nanowire, and enzyme are expressed as a continuous construct. The material binding peptide and nanowire are at the C terminus, which was verified by sequencing. The verified plasmid was transformed into an expression strain of E. coli. Following initial growth of the E. coli, induction was performed and the cultures were permitted to grow for 16-20 hours for recombinant expression. Purified protein was obtained by cell lysis (tip sonication) followed by purification by immobilised metal affinity chromatrography. Identity of the protein was confirmed by SDS-PAGE gel.

### Bio-Electrochemical Formate Production:

Electrode preparation: Graphite felt was cut into 32 mm×32 mm pieces and boiled in deionized water until fully wetted. The wetted graphite felt was then placed at the center of a weighing boat and saturated with the enzyme-linker solution for 10-60 min to deposit the enzyme. After depositing the enzyme, the graphite felt was soaked in buffer solution (100 mM KPi, pH 7.5) for 10 min to wash off the extra enzyme from the electrode surface. The washing step was performed 1-5 times in total with fresh buffer solutions.

Electrochemical measurements: Electrochemical measurements were performed in a custom-made flow electrolyzer (FIG. 12). The enzyme-loaded graphite felt was used as the working electrode. A leak-free Ag/AgCl reference electrode was inserted into the catholyte inlet port of the electrolyzer and was placed at ˜25 mm from the center of the working electrode. The counter electrode was a porous nickel foam. The catholyte was 350 mM NaHCO₃ and the anolyte was 1 M KOH The catholyte vessel was continuously bubbled with CO₂ at 0.2 scfh. The two chambers were divided by a piece of bipolar membrane. The electrolyzer was sealed by PTFE gaskets. The active area of the electrodes was 10 cm². Chronopotentiometry (CP) was performed using a BioLogic SP-150e potentiostat.

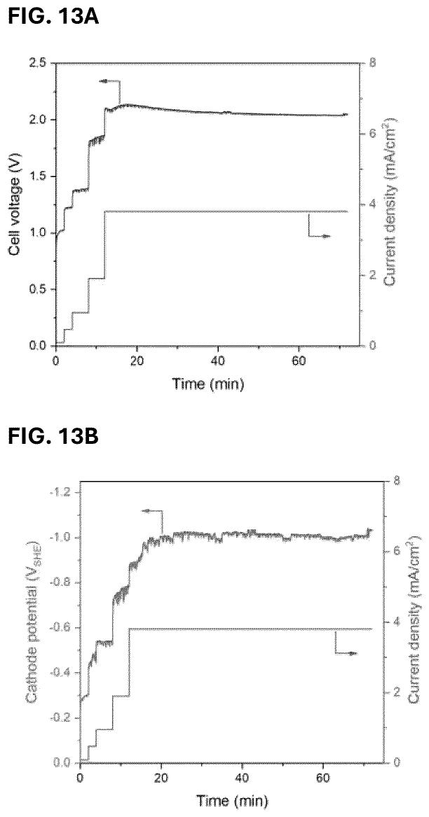

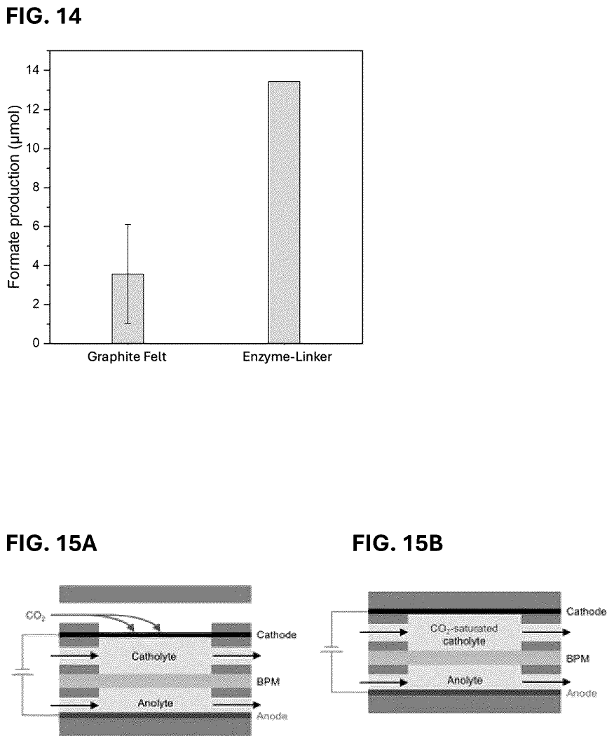

Formate product quantification (FIGS. 13A, 13B, and 14). Formate concentrations in electrolyte were determined by an enzymatic assay. Samples containing formate were combined with NAD+ and a formate dehydrogenase. These were allowed to react under ambient conditions. As formate is consumed a stoichiometric amount of NAD+ is converted to NADH. After reaction completion the concentration of NADH was determined by measuring absorbance at 340 nm and calibration against standards prepared in the same electrolyte matrix as the samples. Each sample and calibration standard was prepared in triplicate and errors reported are the standard deviation from the mean for these replicates.
Faradaic efficiency (FE) towards formate production was calculated by:

$$F E = \frac{c \cdot V \cdot z \cdot F}{Q}$$

in which c is the formate concentration (mol/L), Vis the electrolyte volume (L), z is the number of electron transfer (2), F is the faradaic constant (96,485 C/mol), and Q is the total charge passed (C) during the CP measurement.

### Example 15: Producing Formate with an Enzyme-Linker Absorbed to a Carbon Paper Surface

Enzyme production: The enzyme (Candida boidinii formate dehydrogenase) was expressed recombinantly in E. coli using standard techniques. A plasmid was prepared encoding the enzyme gene, amino acid linker sequence (nanowire GS amino acid sequence), and material binding peptide (MBP) SEQ ID No. 25. The material binding peptide, nanowire, and enzyme are expressed as a continuous construct. The material binding peptide and nanowire are at the C terminus, which was verified by sequencing. The verified plasmid was transformed into an expression strain of E. coli. Following initial growth of the E. coli, induction was performed and the cultures were permitted to grow for 16-20 hours for recombinant expression. Purified protein was obtained by cell lysis (tip sonication) followed by purification by immobilised metal affinity chromatrography. Idenitty of the protein was confirmed by SDS-PAGE gel.

### Bio-Electrochemical Formate Production:

Electrode preparation: Carbon paper was cut into 40 mm×36 mm pieces and boiled in deionized water until fully wetted. The wetted carbon paper was then placed at the center of a weighing boat and saturated with the enzyme-linker for 10-60 min for enzyme deposition. After depositing the enzyme, the carbon paper was taken out and soaked in buffer solution (100 mM KPi, pH 7.5) for 10 min to wash off the excessive enzyme from the electrode surface. The washing step was performed 1-5 in total with fresh buffer solutions.

Electrochemical measurements: Electrochemical measurements were performed in a flow electrolyzer with a two-chamber configuration (FIG. 15A and FIG. 15B). The enzyme-loaded carbon paper was used as the working electrode. The counter electrode was an iridium-containing mixed metal oxide (Ir-MMO). Typically, the catholyte was a NaHCO₃-containing solution and the anolyte was 700 mM NaPi (both 200 mL). The catholyte and anolyte chambers were divided by a piece of bipolar membrane. EPDM gaskets were used for sealing and fixing the active area of the electrodes at 10 cm². There were two ways of delivering CO₂ into the electrolyzer, corresponding to two types of cathode configurations (FIG. 15A and FIG. 15B): 1) Gas-fed system, in which 0.2 scfh of CO₂ was fed through an additional gas chamber on the back of the carbon paper in the flow-by mode; 2) Liquid-fed system, in which CO₂ was continuously bubbled into the external catholyte vessel at 0.2 scfh, and the CO₂-saturated catholyte was supplied to the catholyte chamber. CO₂ reactants in the above two configurations were noted as CO₂(g) and CO₂(aq), respectively. Cyclic voltammetry (CV) and chronopotentiometry (CP) were performed using a BioLogic SP-150e potentiostat.

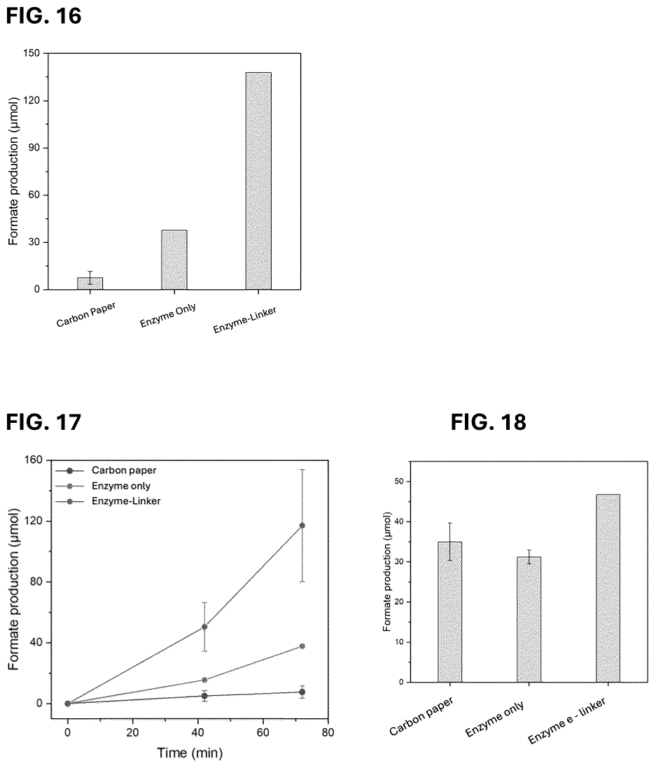

Formate product quantification (FIGS. 16 and 17). Formate concentrations in electrolyte were determined by an enzymatic assay. Samples containing formate were combined with NAD+ and a formate dehydrogenase. These were allowed to react under ambient conditions. As formate is consumed a stoichiometric amount of NAD+ is converted to NADH. After reaction completion the concentration of NADH was determined by measuring absorbance at 340 nm and calibration against standards prepared in the same electrolyte matrix as the samples. Each sample and calibration standard was prepared in triplicate and errors reported are the standard deviation from the mean for these replicates.
Faradaic efficiency (FE) towards formate production was calculated by:

$$F E = \frac{c \cdot V \cdot z \cdot F}{Q}$$

in which c is the formate concentration (mol/L), Vis the electrolyte volume (L), z is the number of electron transfer (2), F is the faradaic constant (96,485 C/mol), and Q is the total charge passed (C) during the CP measurement.

### Example 16: Producing Formate with an Enzyme-Linker Immobilised on a Carbon Paper Surface

Enzyme production: The enzyme (Candida boidinii formate dehydrogenase) was expressed recombinantly in E. coli using standard techniques. A plasmid was prepared encoding the enzyme gene with a surface cystine mutation. The verified plasmid was transformed into an expression strain of E. coli. Following the initial growth of the E. coli, over expression was induced and expression allowed to proceed for 16-20h.Purified protein was obtained by cell lysis (tip sonication) followed by purification by immobilized metal affinity chromatography. Identity of the protein was confirmed by SDS-PAGE gel. To conjugate the produced enzymes with a nanowire and material binding peptide, Purified enzymes were reacted with GrMBP (SEQ ID NO. 25) that has a maleimide moiety installed at the N-terminus. Successful conjugation was confirmed by application of Ellman's assay wherein the free cysteine content was quantified and compared between unreacted enzymes and those reacted with the linker.

### Bio-Electrochemical Formate Production:

Electrode preparation: Carbon paper was cut into 40 mm×36 mm pieces and boiled in deionized water until fully wetted. The wetted carbon paper was then placed at the center of a weigh boat and saturated with the enzyme-linker solution for 10-60 min for enzyme deposition. After depositing the enzyme, the carbon paper was taken out and soaked in buffer solution (100 mM KPi, pH 7.5) for 10 min to wash off the excessive enzyme from the electrode surface. The washing step was performed 1-5 times in total with fresh buffer solutions.

Electrochemical measurements: Electrochemical measurements were performed in a flow electrolyzer with a two-chamber configuration (FIG. 15A and FIG. 15B). The enzyme-loaded carbon paper was used as the working electrode. The counter electrode was an iridium-containing mixed metal oxide (Ir-MMO). Typically, the catholyte was a NaHCO₃-containing solution and the anolyte was 700 mM NaPi (both 200 mL). The catholyte and anolyte chambers were divided by a piece of bipolar membrane. EPDM gaskets were used for sealing and fixing the active area of the electrodes at 10 cm². There were two ways of delivering CO₂ into the electrolyzer, corresponding to two types of cathode configurations (FIG. 15A and FIG. 15B): 1) Gas-fed system, in which 0.2 scfh of CO₂ was fed through an additional gas chamber on the back of the carbon paper in the flow-by mode; 2) Liquid-fed system, in which CO₂ was continuously bubbled into the external catholyte vessel at 0.2 scfh, and the CO₂-saturated catholyte was supplied to the catholyte chamber. CO₂ reactants in the above two configurations were noted as CO₂(g) and CO₂(aq), respectively. Cyclic voltammetry (CV) and chronopotentiometry (CP) were performed using a BioLogic SP-150e potentiostat.

Formate product quantification (FIG. 18): Formate concentrations in electrolyte were determined by an enzymatic assay. Samples containing formate were combined with NAD+ and a formate dehydrogenase. These were allowed to react under ambient conditions. As formate is consumed a stoichiometric amount of NAD+ is converted to NADH. After reaction completion the concentration of NADH was determined by measuring absorbance at 340 nm and calibration against standards prepared in the same electrolyte matrix as the samples. Each sample and calibration standard was prepared in triplicate and errors reported are the standard deviation from the mean for these replicates.
Faradaic efficiency (FE) towards formate production was calculated by:

$$F E = \frac{c \cdot V \cdot z \cdot F}{Q}$$

in which c is the formate concentration (mol/L), Vis the electrolyte volume (L), z is the number of electron transfer (2), F is the faradaic constant (96,485 C/mol), and Q is the total charge passed (C) during the CP measurement.

While preferred embodiments have been described above and illustrated in the accompanying drawings, it will be evident to those skilled in the art that modifications may be made without departing from this disclosure. Such modifications are considered as possible variants comprised in the scope of the disclosure.

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

1. An enzymatic reactor cell, comprising:
a surface, a linker, and one or more enzymes, wherein the surface is directly linked to the linker and the linker is further directly linked to the enzyme, wherein the enzymatic reactor cell produces formic acid and/or formate and/or a salt thereof.

2. The enzymatic reactor cell of claim 1, wherein the one or more enzymes comprises formate dehydrogenase.

3. The enzymatic reactor cell of claim 2, wherein the formate dehydrogenase is a mutant form of formate dehydrogenase.

4. The enzymatic reactor cell of claim 2, wherein the formate dehydrogenase is from Candida boidinii, Myceliophthora thermophila, Thiobacillus sp., Rhodobacter capsulatus, Clostridium ljundahlii, Paraclostridium bifermentans, or from one or more amino acid sequences selected from the group consisting of SEQ ID NOs: 110-13, or SEQ ID NOs: 189-205.

5. The enzymatic reactor cell of claim 1, wherein the enzymatic reactor cell further comprises a starting agent or stating substrate selected from one or more of the groups consisting of gaseous CO2, aqueous CO2, bicarbonate, carbonate, carbonic acid, and/or water.

6. The enzymatic reactor cell of claim 1, wherein the surface is an electrode surface.

7. The enzymatic reactor cell of claim 6, wherein the electrode surface is a cathode.

8. The enzymatic reactor cell of claim 1, wherein the surface is carbon based, an inorganic surface, a metal, a metal oxide, inorganic, silica or silicon, comprises a polymer such as cellulose, polystyrene, PDMS, chlorine doped polypyrrole, polypropylene,
polyethyleneimine, perfluorosulfonic acid polymer, is an alloy and/or is a combination thereof.

9. The enzymatic reactor cell of claim 8, wherein the surface is carbon based and comprises one or more from the following: graphite, graphene, glassy carbon, carbon nanofibers, carbon nanotubes, carbon black, graphene foil, carbon felt, carbon paper, teflonated carbon paper, teflonated carbon felt, carbon based gas diffusion electrode containing a macroporous and/or microporous layer, SWCNT, MWCNT, activated carbon, microporous carbon, hierarchically porous carbon, mesoporous carbon, pyrene, and/or polyethyleneimine.

10. The enzymatic reactor cell of claim 8, wherein the carbon based surface additionally comprises bismuth, bismuth oxide, tin, and/or tin oxide.

11. The enzymatic reactor cell of claim 8, wherein the surface is titanium based.

12. The enzymatic reactor cell of claim 11, wherein the surface is titanium based and additionally comprises bismuth, bismuth oxide, tin, and/or tin oxide.

13. The enzymatic reactor cell of claim 1, wherein the linker comprises a peptide, protein, a chemical polymer, or polynucleotide.

14. The enzymatic reactor cell of claim 13, wherein the linker comprises a surface binding moiety (SBM).

15. The enzymatic reactor cell of claim 14, wherein the surface binding moiety (SBM) binds to or is immobilized to the surface, another portion of the linker, and/or the enzyme by non-covalent bonding, covalent bonding, physisorption, and/or high affinity binding.

16. The enzymatic reactor cell of claim 15, wherein the surface binding moiety (SBM) is covalently bonded to the surface, another portion of the linker, and/or the enzyme.

17. The enzymatic reactor cell of claim 16, wherein the surface binding moiety (SBM) is covalently bonded to the surface, another portion of the linker, and/or the enzyme by click chemistry, dithiol bond formation, Michael addition, nucleophilic substitution, a metal-sulfur bond linkage, a metal-nitrogen bond linkage, a metal-oxygen bond linkage, enzyme catalyzed conjugation, and/or EDC coupling.

18. The enzymatic reactor cell of claim 16, wherein the surface binding moiety (SBM) is covalently bonded by continuous protein expression wherein the surface binding moiety (SBM) is a continuous protein with another portion of the linker; the surface binding moiety (SBM) is a continuous protein with the enzyme; or the surface binding moiety (SBM) is a continuous protein with another portion of the linker, and/or the enzyme.

19. The enzymatic reactor cell of claim 1, wherein the linker binds to another portion of the linker, the enzyme, and/or the surface.

20. The enzymatic reactor cell of claim 19, wherein the linker is covalently bonded to another portion of the linker, the enzyme, and/or the surface by click chemistry, dithiol bond formation, Michael addition, nucleophilic substitution, a metal-sulfur bond linkage, a metal-nitrogen bond linkage, a metal-oxygen bond linkage, enzyme catalyzed conjugation, and/or EDC coupling.

21. The enzymatic reactor cell of claim 14, wherein the SBM comprises a material binding peptide (MBP).

22. The enzymatic reactor cell of claim 21, wherein the material binding peptide (MBP) or surface binding moiety (SBM) of the linker comprises one or more amino acid sequences selected from the group consisting of SEQ ID NOs: 1-33, SEQ ID NO: 50, SEQ ID NOs: 54-105, and SEQ ID Nos. 124-171.

23. The enzymatic reactor cell of claim 21, wherein the MBP comprises SEQ ID NO. 25.

24. The enzymatic reactor cell of claim 21, wherein the material binding peptide (MBP) binds to carbon based surfaces, metal, metal oxide, a polymer and/or an inorganic surface.

25. The enzymatic reactor cell of claim 1, wherein the linker comprises a peptide, protein, a chemical polymer, nanowire, polynucleotide or chemical means such as chemical nanowires to link a) portions of the linker together, b) the linker to the surface and/or c) the linker to the enzyme.

26. The enzymatic reactor cell of claim 25, wherein the chemical means or chemical nanowire is used for linking, or included a maleimide functional group, a tetrafluorophenyl functional group, an aldehyde functional group, an amine functional group, a N-hydroxysuccinimide functional group, a thiol functional group, a haloacetyl functional group, a pyridyl disulfide functional group, an imidoester functional group, an epoxide functional group, a hydrocarbon chain, one or more polyethylene glycol units, and/or one or more aromatic rings.

27. The enzymatic reactor cell of claim 26, wherein the nanowire comprises or uses maleimide.

28. The enzymatic reactor cell of claim 25, wherein the linker comprises a peptide or protein.

29. The enzymatic reactor cell of claim 28, wherein the peptide comprises one or more amino acid sequences selected from the group consisting of SEQ ID Nos: 34-51, SEQ ID Nos: 172-188 and SEQ ID No: 206.

30. A method of performing an enzymatic reaction or enzymatic pathway with the use of an enzymatic reactor cell of claim 1 or a plurality of enzymatic reactor cells of claim 1.


SEQUENCE LISTING (206 sequences)

SEQ ID NO: 1 - protein, 42 aa, synthetic construct
MHGKTQATSGTIQSMHGKTQATSGTIQSMHGKTQATSGTIQS

SEQ ID NO: 2 - protein, 98 aa, synthetic construct
MHGKTQATSGTIQSMHGKTQATSGTIQSMHGKTQATSGTIQSMHGKTQATSGTIQSMHGKTQATSGTIQSMHGKTQATSGTIQSMHGKTQATSGTIQS

SEQ ID NO: 3 - protein, 12 aa, synthetic construct
WAGAKRLVLRRE

SEQ ID NO: 4 - protein, 10 aa, synthetic construct
HFSSWETQQG

SEQ ID NO: 5 - protein, 10 aa, synthetic construct
WYEKWQKANW

SEQ ID NO: 6 - protein, 8 aa, synthetic construct
VSGSSPDS

SEQ ID NO: 7 - protein, 36 aa, synthetic construct
SSKKSGSYSGSKGSRRILGGGGMHGKTQATSGTIQS

SEQ ID NO: 8 - protein, 30 aa, synthetic construct
MSPHPHPRHHHTGGGGMHGKTQATSGTIQS

SEQ ID NO: 9 - protein, 30 aa, synthetic construct
RGRRRRLSCRLLGGGGMHGKTQATSGTIQS

SEQ ID NO: 10 - protein, 30 aa, synthetic construct
DSARGFKKPGKRGGGGMHGKTQATSGTIQS

SEQ ID NO: 11 - protein, 30 aa, synthetic construct
HPPMNASHPHMHGGGGMHGKTQATSGTIQS

SEQ ID NO: 12 - protein, 30 aa, synthetic construct
HKDHHANQHVHMGGGGMHGKTQATSGTIQS

SEQ ID NO: 13 - protein, 16 aa, synthetic construct
HPPMNASHPHMHGGGG

SEQ ID NO: 14 - protein, 108 aa, synthetic construct
PTTGSCAVTYTANGWSGGFTAAVTLTNTGTTALSGWTLGFAFPSGQTLTQGWSARWAQSGSSVTATNEAWNAVLAPGASVEIGFSGTHTGTNTAPATFTVGGATCTTR

SEQ ID NO: 15 - protein, 108 aa, synthetic construct
SGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVG

SEQ ID NO: 16 - protein, 12 aa, synthetic construct
RAFIASRRIRRP

SEQ ID NO: 17 - protein, 8 aa, synthetic construct
RIIIRRIR

SEQ ID NO: 18 - protein, 12 aa, synthetic construct
RGRRRRLSCRLL

SEQ ID NO: 19 - protein, 18 aa, synthetic construct
SSKKSGSYSGSKGSRRIL

SEQ ID NO: 20 - protein, 12 aa, synthetic construct
MSPHPHPRHHHT

SEQ ID NO: 21 - protein, 12 aa, synthetic construct
DSARGFKKPGKR

SEQ ID NO: 22 - protein, 12 aa, synthetic construct
HPPMNASHPHMH

SEQ ID NO: 23 - protein, 12 aa, synthetic construct
HKDHHANQHVHM

SEQ ID NO: 24 - protein, 12 aa, synthetic construct
NPSSLFRYLPSD

SEQ ID NO: 25 - protein, 12 aa, synthetic construct
HSSYWYAFNNKT

SEQ ID NO: 26 - protein, 12 aa, synthetic construct
EAHVMHKVAPRP

SEQ ID NO: 27 - protein, 12 aa, synthetic construct
HHGHSPTSPQVR

SEQ ID NO: 28 - protein, 30 aa, synthetic construct
MVMPGDNIKMVVTLIHPIAMDDGLRFAIRE

SEQ ID NO: 29 - protein, 45 aa, synthetic construct
VGPNNVPYIVATITSNSAGGQPVSLANLKAMYSIAKKYDIPVVMD

SEQ ID NO: 30 - protein, 31 aa, synthetic construct
NNSWTRVAFAGLKFQDVGSFDYGRNYGVVYD

SEQ ID NO: 31 - protein, 120 aa, synthetic construct
QVQLVESGAEVKKPGESLKISCKGSGYSFPSYWINWVRQMPGKGLEWMGMIYPADSDTRYSPSFQGHVTISADKSINTAYLQWAGLKASDTAIYYCARLGIGGRYMSRWGQGTLVTVSSA

SEQ ID NO: 32 - protein, 10 aa, synthetic construct
LEYKRGYKPR

SEQ ID NO: 33 - protein, 20 aa, synthetic construct
DYKDDDDKAYSSGAPPMPPF

SEQ ID NO: 34 - protein, 61 aa, synthetic construct
FTLIELLIVVAIIGILAAIAIPQFSAYRVKAYNSAASSDLRNLKTALESAFADDQTYPPES

SEQ ID NO: 35 - protein, 144 aa, synthetic construct
FTLIELMIVVAIIGILAAIAIPQYQNYVARSEGASALASVNPLKTTVEEALSRGWSVKSGTGTEDATKKEVPLGVAADANKLGTIALKPDPADGTADITLTFTMGGAGPKNKGKIITLTRTAADGLWKCTSDQDEQFIPKGCSR

SEQ ID NO: 36 - protein, 167 aa, synthetic construct
MASNFKFKLLSQLKKRAEGGFTLIELLVVVIIIGVLAAIALPNLLGQVGKARESEAKSTIGALNRAQQGYFTEKGTFATDTETLEVPAPDGNFFSFAVNTADNTEAIQDATALNWEADGTRSMSGGTFYDSGTRAFSTVVCRAEAGSEDTPPTPGGANDCGGAEVIK

SEQ ID NO: 37 - protein, 28 aa, synthetic construct
ELKAIAQEFKAIAKEFAIAFEFKAIAQK

SEQ ID NO: 38 - protein, 7 aa, synthetic construct
YYACAYY

SEQ ID NO: 39 - protein, 7 aa, synthetic construct
GNNQQNY

SEQ ID NO: 40 - protein, 9 aa, synthetic construct
KVQIINKKL

SEQ ID NO: 41 - protein, 5 aa, synthetic construct
VGGLG

SEQ ID NO: 42 - protein, 8 aa, synthetic construct
VGGLGHHH

SEQ ID NO: 43 - protein, 8 aa, synthetic construct
VGGLGWWW

SEQ ID NO: 44 - protein, 8 aa, synthetic construct
VGGLGYYY

SEQ ID NO: 45 - protein, 7 aa, synthetic construct
VGGLFFF

SEQ ID NO: 46 - protein, 10 aa, synthetic construct
SVNVTQVGFP

SEQ ID NO: 47 - protein, 13 aa, synthetic construct
SVNVTQVGFPHHH

SEQ ID NO: 48 - protein, 13 aa, synthetic construct
SVNVTQVGFPWWW

SEQ ID NO: 49 - protein, 13 aa, synthetic construct
SVNVTQVGFPYYY

SEQ ID NO: 50 - protein, 6 aa, synthetic construct
RKLPDA

SEQ ID NO: 51 - protein, 9 aa, synthetic construct
PPPYPPPWC

SEQ ID NO: 52-53 - skipped (2 entries)

SEQ ID NO: 54 - protein, 7 aa, synthetic construct
EPLQLKM

SEQ ID NO: 55 - protein, 12 aa, synthetic construct
GAMHLPWHMGTL

SEQ ID NO: 56 - protein, 12 aa, synthetic construct
IMVTASSAYRRY

SEQ ID NO: 57 - protein, 16 aa, synthetic construct
VIAGASLWWSEKLVIA

SEQ ID NO: 58 - protein, 13 aa, synthetic construct
CALNNDEVDKFAM

SEQ ID NO: 59 - protein, 13 aa, synthetic construct
KKNYSSSISSIHC

SEQ ID NO: 60 - protein, 15 aa, synthetic construct
CGGHSSKLQFWYFWY

SEQ ID NO: 61 - protein, 12 aa, synthetic construct
HWSAWWIRSNQS

SEQ ID NO: 62 - protein, 12 aa, synthetic construct
IMVTESSDYSSY

SEQ ID NO: 63 - protein, 12 aa, synthetic construct
IMVTASSAYDDY

SEQ ID NO: 64 - protein, 12 aa, synthetic construct
IMVTQSSNYSSY

SEQ ID NO: 65 - protein, 12 aa, synthetic construct
IMVTKSSDYSSY

SEQ ID NO: 66 - protein, 12 aa, synthetic construct
HSSAAAAFNNKT

SEQ ID NO: 67 - protein, 12 aa, synthetic construct
HTSYWYAFNTKT

SEQ ID NO: 68 - protein, 12 aa, synthetic construct
YTTHVLPFAPSS

SEQ ID NO: 69 - protein, 11 aa, synthetic construct
HAWVDWIRPIH

SEQ ID NO: 70 - protein, 12 aa, synthetic construct
HWKHPWGAWDTL

SEQ ID NO: 71 - protein, 7 aa, synthetic construct
QQQLSTH

SEQ ID NO: 72 - protein, 18 aa, synthetic construct
GGPDSARGFKKPGKRGPC

SEQ ID NO: 73 - protein, 15 aa, synthetic construct
GGGMHPNAGHGSLMR

SEQ ID NO: 74 - protein, 36 aa, synthetic construct
TPQSHYGQCGGGYSGPTVCASGTTCQVLNPYYSQCL

SEQ ID NO: 75 - protein, 157 aa, synthetic construct
GVVSVQFNNGSSPASSNSIYARFKVTNTSGSPINLADLKLRYYYTQDADKPLTFWCDHAGYMSGSNYIDATSKVTGSFKAVSPAVTNADHYLEVALNSDAGSLPAGGSIEIQTRFARNDWSNFDQSNDWSYTAAGSYMDWQKISAFVGGTLAYGSTP

SEQ ID NO: 76 - protein, 12 aa, synthetic construct
QPYLFATDSLIK

SEQ ID NO: 77 - protein, 12 aa, synthetic construct
GHTHYHAVRTQT

SEQ ID NO: 78 - protein, 9 aa, synthetic construct
RLLLRRLRR

SEQ ID NO: 79 - protein, 13 aa, synthetic construct
KRAFIASRRIRRP

SEQ ID NO: 80 - skipped

SEQ ID NO: 81 - protein, 12 aa, synthetic construct
HGGGHGHGGGHG

SEQ ID NO: 82 - protein, 12 aa, synthetic construct
TSNAVHPTLRHL

SEQ ID NO: 83 - protein, 12 aa, synthetic construct
NFMSLPRLGHMH

SEQ ID NO: 84 - protein, 9 aa, synthetic construct
AHHAHHAAD

SEQ ID NO: 85 - protein, 7 aa, synthetic construct
SGTGASY

SEQ ID NO: 86 - protein, 8 aa, synthetic construct
DAHKSEVA

SEQ ID NO: 87 - protein, 6 aa, synthetic construct
SYHHHH

SEQ ID NO: 88 - protein, 12 aa, synthetic construct
ERSWTLDSALSM

SEQ ID NO: 89 - protein, 12 aa, synthetic construct
VPSSGPQDTRTT

SEQ ID NO: 90 - protein, 12 aa, synthetic construct
YSPDPRPWSSRY

SEQ ID NO: 91 - protein, 12 aa, synthetic construct
NLNPNTASAMHV

SEQ ID NO: 92 - protein, 12 aa, synthetic construct
VQHNTKYSVVIR

SEQ ID NO: 93 - protein, 12 aa, synthetic construct
VQHNTKYSVVIR

SEQ ID NO: 94 - protein, 12 aa, synthetic construct
MTWDPSLASPRS

SEQ ID NO: 95 - protein, 17 aa, synthetic construct
ACTSNADNKYLPKTCQT

SEQ ID NO: 96 - protein, 9 aa, synthetic construct
RRTVKHHVN

SEQ ID NO: 97 - protein, 12 aa, synthetic construct
QMDTSTSLAPSR

SEQ ID NO: 98 - protein, 9 aa, synthetic construct
AHHAHHAAD

SEQ ID NO: 99 - protein, 9 aa, synthetic construct
CHKKPSKSC

SEQ ID NO: 100 - protein, 12 aa, synthetic construct
THRTSTLDYFVI

SEQ ID NO: 101 - protein, 12 aa, synthetic construct
TSDIKSRSPHHR

SEQ ID NO: 102 - protein, 12 aa, synthetic construct
HTQNMRMYEPWF

SEQ ID NO: 103 - protein, 47 aa, synthetic construct
AIKLVQSPNGNFAASFVLDGTKWIFKSKYYDSSKGYWVGIYEVWDRK

SEQ ID NO: 104 - protein, 47 aa, synthetic construct
AIKLVQSPNGNFAASFVLDGTKWTFKSKHYDSSKGYWVGIYKVWDRK

SEQ ID NO: 105 - protein, 47 aa, synthetic construct
AIKLVQSPNGNFAASFVLDGTKWIFKSKRYDSSKGYWVGIYEVWRRK

SEQ ID NO: 106 - DNA, 13 bp, synthetic construct
  source 1..13: mol_type = other DNA
gcaactaggctcg

SEQ ID NO: 107 - DNA, 13 bp, synthetic construct
  source 1..13: mol_type = other DNA
cgagcctagttgc

SEQ ID NO: 108 - DNA, 22 bp, synthetic construct
  source 1..22: mol_type = other DNA
gcacctgaaccgcatggactcg

SEQ ID NO: 109 - DNA, 22 bp, synthetic construct
  source 1..22: mol_type = other DNA
cgagtccatgcggttcaggtgc

SEQ ID NO: 110 - protein, 377 aa, synthetic construct
MHHHHHHENLYFQMKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYITKAYGKHDKK

SEQ ID NO: 111 - protein, 364 aa, Candida boidinii
MKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYITKAYGKHDKK

SEQ ID NO: 112 - protein, 370 aa, synthetic construct
MKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKKHHHHHH

SEQ ID NO: 113 - protein, 377 aa, synthetic construct
MAHHHHHHVGMVKVLAVLYDGGKHAQQVPGLLGTTENELGLRKWLEDQGHTLVTTSDKDGENSTFDRELVDAEIIITTPFHPGYLTAERLAKAKKLKLAVTAGIGSDHVDLDAANKTNGGITVAEVTGSNVVSVAEHVVMTILVLVRNFVPAHEQIEAGRWDVAEAAKNEFDLEGKVVGTVAVGRIGERVLRRLKAFDCKELLYYDYQPLSPEKEKEIGCRRVLDLEEMLGQCDVVTINCPLHEKTRGLFNKDLIAKMKPGSWLVNTARGAIVVKEDVAEALRTGHLRGYGGDVWFPQPAPADHPLRTAKNPFGGGNAMVPHMSGTSLDAQKRYADGVKRILESYLSGRFDYRPEDLIVHNGQYATRSYGQREVSTS

SEQ ID NO: 114 - protein, 371 aa, Myceliophthora thermophila
MAVGMVKVLAVLYDGGKHAQQVPGLLGTTENELGLRKWLEDQGHTLVTTSDKDGENSTFDRELVDAEIIITTPFHPGYLTAERLAKAKKLKLAVTAGIGSDHVDLDAANKTNGGITVAEVTGSNVVSVAEHVVMTILVLVRNFVPAHEQIEAGRWDVAEAAKNEFDLEGKVVGTVAVGRIGERVLRRLKAFDCKELLYYDYQPLSPEKEKEIGCRRVLDLEEMLGQCDVVTINCPLHEKTRGLFNKDLIAKMKPGSWLVNTARGAIVVKEDVAEALRTGHLRGYGGDVWFPQPAPADHPLRTAKNPFGGGNAMVPHMSGTSLDAQKRYADGVKRILESYLSGRFDYRPEDLIVHNGQYATRSYGQREVSTS

SEQ ID NO: 115 - protein, 407 aa, synthetic construct
MAKILCVLYDDPVDGYPKTYARDDLPKIDHYPGGQTLPTPKAIDFTPGQLLGSVSGELGLRKYLEANGHTFVVTSDKDGPDSVFEKELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAGHHHHHH

SEQ ID NO: 116 - protein, 401 aa, Thiobacillus sp.
MAKILCVLYDDPVDGYPKTYARDDLPKIDHYPGGQTLPTPKAIDFTPGQLLGSVSGELGLRKYLEANGHTFVVTSDKDGPDSVFEKELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAG

SEQ ID NO: 117 - protein, 165 aa, synthetic construct
MHHHHHHENLYFQGSMTDTARLRAILAAHRGREGALLPILHDVQAAFGFIPEDAYAPIAADLGLTRAEVAGVVGFYHDFRKAPAGRHVIKLCRAEACQAMGMDAVQARLESALGLRLGDSSEAVTLEAVYCLGLCACAPAAMVDDRLVGRLDAAAVAGIVAELGA

SEQ ID NO: 118 - protein, 500 aa, Rhodobacter capsulatus
MKIWLPCDAAAKACGAEAVLAALRLEAEKRGGALDIARNGSRGMIWLEPLLEVETPAGRIGFGPMTPADVPALFDALESHPKALGLVEEIPFFKRQTRLTFARCGRIEPLSLAQFAAAEGWAGLRKALKMTPAEVVEEVLASGLRGRGGAGFPTGIKWRTVAAAQADQKYIVCNVDEGDSGSFADRMLIEGDPFCLVEGMAIAGHAVGATRGYVYIRSEYPDAIAVMRAAIAMAKPFLAEAGFEMEVRVGAGAYVCGEETSLLNSLEGKRGTVRAKPPLPALKGLFGKPTVVNNLLSLAAVPWIIAHGAKAYESFGMDRSRGTIPLQIGGNVKRGGLFETGFGITLGELVEDICGGTASGRPVKAVQVGGPLGAYHPVSDYHLPFCYEQFAGQGGLVGHAGLVVHDDTADMLKLARFAMEFCAIESCGTCTPCRIGAVRGVEVIDRIAAGDASAMPLLDDLCQTMKLGSLCALGGFTPYPVQSAIRHFPADFPCAREAAE

SEQ ID NO: 119 - protein, 958 aa, Rhodobacter capsulatus
MKDLIIPPLDWTQDMGTPKREGAPVHLTIDGVEVTVPAGTSVLRAAAEAGISIPKLCATDNVEPVGSCRLCMVEIEGMRGTPTSCTTPVAPGMRVHTQTPQLQKLRRGVMELYISDHPLDCLTCAANGDCELQDMAGAVGLREVRYQAKDTHFARRDATGPNPRYIPKDNSNPYFSYDPAKCIVCMRCVRACEEVQGTFALTVMGRGFDARISPAAPDFLSSDCVSCGACVQACPTATLVEKSVERIGTPERKVVTTCAYCGVGCSFEAHMLGDQLVRMVPWKGGAANRGHSCVKGRFAYGYATHQDRILKPMIRDKITDPWREVNWTEALDFTATRLRALRDSHGADALGVITSSRCTNEETYLVQKLARAVFGTNNTDTCARVCHSPTGYGLKQTFGTSAGTQDFDSVEETDLALVIGANPTDGHPVFASRLRKRLRAGAKLIVVDPRRIDLLNTPHRGEAWHLQLKPGTNVAVMTAMAHVIVTEQIFDKRFIGDRCDWDEWADYAEFVANPEYAPEAVESLTGVPAGLLRQAARAYAAAPNAAIYYGLGVTEHSQGSTTVIAIANLAMMTGNIGRPGVGVNPLRGQNNVQGSCDMGSFPHEFPGYRHVSDDATRGLFERTWGVTLSSEPGLRIPNMLDAAVEGRFKALYVQGEDILQSDPDTRHVSAGLAAMDLVIVHDLFLNETANYAHVFLPGSTFLEKDGTFTNAERRINRVRRVMAPKAGFADWEVTQMLANALGAGWHYTHPSEIMAEIAATTPGFAAVTYEMLDARGSVQWPCNEKAPEGSPIMHVEGFVRGKGRFIRTAYLPTDEKTGPRFPLLLTTGRILSQYNVGAQTRRTENTVWHGEDRLEIHPTDAETRGIRDGDWVRLASRAGETTLRATVTDRVSPGVVYTTFHHPDTQANVVTTDTSDWATNCPEYKVTAVQVAASNGPSDWQQDYAAQAAAARRIEAAE

SEQ ID NO: 120 - protein, 255 aa, Rhodobacter capsulatus
MSLPAGAVTVPLPGGARAVLAEEVPVALVFDGVTQAVMMASPVDLEDFLLGFALTEGMIADRAELLRHEVVRQPQGIELRGWLAAPAGQRFAARRRAMAGPVGCGLCGLDSLAAVLRPLPRAPRGGAPPPLADGALAALRAGQSLQDAVRSVHAAGFWDGAQMRALREDVGRHNALDKLAGALAGQGIDAAAGALVLTSRLSVDLVQKAAMIGARVLIAPSAPTALAVAEAQAAGLALIARGPDGPTLYTETEAE

SEQ ID NO: 121 - protein, 79 aa, synthetic construct
MSDDKIIRMANQIAAFFAVQPGDRAGPVAAHISENWSAPMRAALLAHVAAQSPGLDPLVIAAAPQIRPVPALEHHHHHH

SEQ ID NO: 122 - protein, 153 aa, Rhodobacter capsulatus
MGSMTDTARLRAILAAHRGREGALLPILHDVQAAFGFIPEDAYAPIAADLGLTRAEVAGVVGFYHDFRKAPAGRHVIKLCRAEACQAMGMDAVQARLESALGLRLGDSSEAVTLEAVYCLGLCACAPAAMVDDRLVGRLDAAAVAGIVAELGA

SEQ ID NO: 123 - protein, 73 aa, Rhodobacter capsulatus
MSDDKIIRMANQIAAFFAVQPGDRAGPVAAHISENWSAPMRAALLAHVAAQSPGLDPLVIAAAPQIRPVPALE

SEQ ID NO: 124 - protein, 7 aa, synthetic construct
LPPWKLK

SEQ ID NO: 125 - protein, 7 aa, synthetic construct
WSLSELH

SEQ ID NO: 126 - protein, 7 aa, synthetic construct
IGASVHR

SEQ ID NO: 127 - protein, 7 aa, synthetic construct
AHHLKVS

SEQ ID NO: 128 - protein, 7 aa, synthetic construct
NHPLYNR

SEQ ID NO: 129 - protein, 7 aa, synthetic construct
ALEHTSR

SEQ ID NO: 130 - protein, 7 aa, synthetic construct
HPAIRPP

SEQ ID NO: 131 - protein, 7 aa, synthetic construct
LHRHANL

SEQ ID NO: 132 - protein, 7 aa, synthetic construct
SSNQFHQ

SEQ ID NO: 133 - protein, 7 aa, synthetic construct
KVPGHQQ

SEQ ID NO: 134 - protein, 7 aa, synthetic construct
TLAPRTA

SEQ ID NO: 135 - protein, 7 aa, synthetic construct
VGKTHAD

SEQ ID NO: 136 - protein, 7 aa, synthetic construct
FPLHELR

SEQ ID NO: 137 - protein, 4 aa, synthetic construct
LPPW

SEQ ID NO: 138 - protein, 4 aa, synthetic construct
WKLK

SEQ ID NO: 139 - protein, 12 aa, synthetic construct
KNAGQYPPSALM

SEQ ID NO: 140 - protein, 12 aa, synthetic construct
SPSHSADHTPPT

SEQ ID NO: 141 - protein, 12 aa, synthetic construct
TPTLRSMSSLLF

SEQ ID NO: 142 - protein, 12 aa, synthetic construct
STLTQSTSSLVA

SEQ ID NO: 143 - protein, 12 aa, synthetic construct
INTKNYALRTLH

SEQ ID NO: 144 - protein, 12 aa, synthetic construct
KLHISKDHIYPT

SEQ ID NO: 145 - protein, 12 aa, synthetic construct
SWMPHPRRSPGH

SEQ ID NO: 146 - protein, 12 aa, synthetic construct
SSYYPGLTAHRF

SEQ ID NO: 147 - protein, 12 aa, synthetic construct
GAMHLPWHMGTL

SEQ ID NO: 148 - protein, 12 aa, synthetic construct
VRCIIRGIVGTH

SEQ ID NO: 149 - protein, 12 aa, synthetic construct
LGCLIRVGVLQH

SEQ ID NO: 150 - protein, 12 aa, synthetic construct
YTKPTENIHQRA

SEQ ID NO: 151 - protein, 12 aa, synthetic construct
WSRIIRQASYTQ

SEQ ID NO: 152 - protein, 12 aa, synthetic construct
TYAKGHHSWPPP

SEQ ID NO: 153 - protein, 12 aa, synthetic construct
SGMPHPRRSPQH

SEQ ID NO: 154 - protein, 12 aa, synthetic construct
TIMGETFAMPAS

SEQ ID NO: 155 - protein, 12 aa, synthetic construct
ETLHISTHPNET

SEQ ID NO: 156 - protein, 12 aa, synthetic construct
GIGLIRGRSQEL

SEQ ID NO: 157 - protein, 12 aa, synthetic construct
LLWVRLLLCSAS

SEQ ID NO: 158 - protein, 12 aa, synthetic construct
GGMHIPWHMGPL

SEQ ID NO: 159 - protein, 12 aa, synthetic construct
GGKHNPRHTDPR

SEQ ID NO: 160 - protein, 12 aa, synthetic construct
KHMHNLTPQWTK

SEQ ID NO: 161 - protein, 12 aa, synthetic construct
SGHHNLHKTEHR

SEQ ID NO: 162 - protein, 12 aa, synthetic construct
GHHHSKTFETQT

SEQ ID NO: 163 - protein, 12 aa, synthetic construct
GHSMHRPLKLFH

SEQ ID NO: 164 - protein, 12 aa, synthetic construct
GLSMHRPLKLFH

SEQ ID NO: 165 - protein, 12 aa, synthetic construct
KPHHNWESAYSK

SEQ ID NO: 166 - protein, 13 aa, synthetic construct
VMNHWQEDLMFGY

SEQ ID NO: 167 - protein, 12 aa, synthetic construct
PENLLYYHPGTD

SEQ ID NO: 168 - protein, 12 aa, synthetic construct
TGTPAMVDANNG

SEQ ID NO: 169 - protein, 13 aa, synthetic construct
YSENEEIVGLAAK

SEQ ID NO: 170 - protein, 12 aa, synthetic construct
TCFFRDHSYQEE

SEQ ID NO: 171 - protein, 10 aa, synthetic construct
IDEITDINNT

SEQ ID NO: 172 - protein, 16 aa, synthetic construct
  REPEAT 1..2: note = single repeat of GS
  VARIANT 3..16: note = up to 7 copies of GS can be deleted
GSGSGSGSGSGSGSGS

SEQ ID NO: 173 - protein, 8 aa, synthetic construct
VDGGGGGS

SEQ ID NO: 174 - protein, 14 aa, synthetic construct
GTGGGGVDGGGGGS

SEQ ID NO: 175 - protein, 4 aa, synthetic construct
GGGG

SEQ ID NO: 176 - protein, 5 aa, synthetic construct
GGGGG

SEQ ID NO: 177 - protein, 6 aa, synthetic construct
GGGGGG

SEQ ID NO: 178 - protein, 8 aa, synthetic construct
GGGGGGGG

SEQ ID NO: 179 - protein, 15 aa, synthetic construct
  REPEAT 1..3: note = single repeat of GGS
  VARIANT 4..15: note = up to 4 copies of GGS can be deleted
GGSGGSGGSGGSGGS

SEQ ID NO: 180 - protein, 16 aa, synthetic construct
  REPEAT 1..4: note = single repeat of GGGS
  VARIANT 5..16: note = up to 3 copies of GGGS can be deleted
GGGSGGGSGGGSGGGS

SEQ ID NO: 181 - protein, 5 aa, synthetic construct
PAPAP

SEQ ID NO: 182 - protein, 20 aa, synthetic construct
  REPEAT 1..5: note = single repeat of EAAAK
  VARIANT 6..20: note = up to copies of EAAAK can be deleted
EAAAKEAAAKEAAAKEAAAK

SEQ ID NO: 183 - protein, 5 aa, synthetic construct
GGYGG

SEQ ID NO: 184 - protein, 5 aa, synthetic construct
GGFGG

SEQ ID NO: 185 - protein, 6 aa, synthetic construct
GGFFGG

SEQ ID NO: 186 - protein, 6 aa, synthetic construct
GGYYGG

SEQ ID NO: 187 - protein, 8 aa, synthetic construct
GGSYWGGS

SEQ ID NO: 188 - protein, 19 aa, synthetic construct
  REPEAT 6..10: note = single repeat of EAAAK
  VARIANT 11..15: note = up to 1 copy of EAAAK can be deleted
GGGGSEAAAKEAAAKGGGS

SEQ ID NO: 189 - protein, 370 aa, synthetic construct
MKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKKHHHHHH

SEQ ID NO: 190 - protein, 385 aa, synthetic construct
MAHHHHHHKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKKGSHSSYWYAFNNKT

SEQ ID NO: 191 - protein, 370 aa, synthetic construct
  VARIANT 10: note = C or A
  VARIANT 23: note = C or S
  VARIANT 121: note = V or C
  VARIANT 151: note = E or C
  VARIANT 201: note = K or C
  VARIANT 241: note = K or C
  VARIANT 272: note = E or C
  VARIANT 273: note = S or C
  VARIANT 318: note = D or C
  VARIANT 321: note = T or C
MKIVLVLYDXGKHAADEEKLYGXTENKLGIANWLKDQGHELITTSDKEGGNSVLDQHIPDADIIITTPFHPAYITKERIDKAKKLKLVVVAGVGSDHIDLDYINQTGKKISVLEVTGSNVXSVAEHVLMTMLVLVRNFVPAHEQIINHDWXVAAIAKDAYDIEGKTIATIGAGRIGYRVLERLVPFNPKELLYYDYQALPXDAEEKVGARRVENIEELVAQADIVTINAPLHAGTKGLINXELLSKFKKGAWLVNTARGAICVAEDVAAALXXGQLRGYGGDVWSPQPAPKDHPWRDMRNKYGAGNAMTPHYSGTTLXAQXRYAEGTKNILESFFTGKFDYRPQDIILLNGEYITKAYGKHDKKHHHHHH

SEQ ID NO: 192 - protein, 364 aa, Candida boidinii
MKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKK

SEQ ID NO: 193 - protein, 371 aa, synthetic construct
MAHHHHHHKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKK

SEQ ID NO: 194 - protein, 378 aa, synthetic construct
MKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKKGSHSSYWYAFNNKT

SEQ ID NO: 195 - protein, 386 aa, synthetic construct
MHSSYWYAFNNKTGSKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKKLEHHHHHH

SEQ ID NO: 196 - protein, 380 aa, synthetic construct
MHSSYWYAFNNKTGSKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYITKAYGKHDKKLE

SEQ ID NO: 197 - protein, 371 aa, synthetic construct
  VARIANT 17: note = C or A
  VARIANT 30: note = C or S
  VARIANT 128: note = V or C
  VARIANT 158: note = E or C
  VARIANT 208: note = K or C
  VARIANT 248: note = K or C
  VARIANT 279: note = E or C
  VARIANT 280: note = S or C
  VARIANT 325: note = D or C
  VARIANT 328: note = T or C
MAHHHHHHKIVLVLYDXGKHAADEEKLYGXTENKLGIANWLKDQGHELITTSDKEGGNSVLDQHIPDADIIITTPFHPAYITKERIDKAKKLKLVVVAGVGSDHIDLDYINQTGKKISVLEVTGSNVXSVAEHVLMTMLVLVRNFVPAHEQIINHDWXVAAIAKDAYDIEGKTIATIGAGRIGYRVLERLVPFNPKELLYYDYQALPXDAEEKVGARRVENIEELVAQADIVTINAPLHAGTKGLINXELLSKFKKGAWLVNTARGAICVAEDVAAALXXGQLRGYGGDVWSPQPAPKDHPWRDMRNKYGAGNAMTPHYSGTTLXAQXRYAEGTKNILESFFTGKFDYRPQDIILLNGEYITKAYGKHDKK

SEQ ID NO: 198 - protein, 364 aa, synthetic construct
  VARIANT 10: note = C or A
  VARIANT 23: note = C or S
  VARIANT 121: note = V or C
  VARIANT 151: note = E or C
  VARIANT 201: note = K or C
  VARIANT 241: note = K or C
  VARIANT 272: note = E or C
  VARIANT 273: note = S or C
  VARIANT 318: note = D or C
  VARIANT 321: note = T or C
MKIVLVLYDXGKHAADEEKLYGXTENKLGIANWLKDQGHELITTSDKEGGNSVLDQHIPDADIIITTPFHPAYITKERIDKAKKLKLVVVAGVGSDHIDLDYINQTGKKISVLEVTGSNVXSVAEHVLMTMLVLVRNFVPAHEQIINHDWXVAAIAKDAYDIEGKTIATIGAGRIGYRVLERLVPFNPKELLYYDYQALPXDAEEKVGARRVENIEELVAQADIVTINAPLHAGTKGLINXELLSKFKKGAWLVNTARGAICVAEDVAAALXXGQLRGYGGDVWSPQPAPKDHPWRDMRNKYGAGNAMTPHYSGTTLXAQXRYAEGTKNILESFFTGKFDYRPQDIILLNGEYITKAYGKHDKK

SEQ ID NO: 199 - protein, 709 aa, Clostridium ljundahlii
MDKKVLTVCPYCGAGCNLYLHVKNGKIIKAEPANGRTNEGSLCLKGHFGWDFLNDPKILTSRIKHPMIRKNGELEEVSWDEAISFTASRLSQIKEKYGPDSIMGTGCARGSGNEANYIMQKFMRAVIGTNNVDHCARVCHAPSVAGLAYVLGNGAMSNGIHEIDDTDLLLIFGYNGAASHPIVAKRIVRAKQKGAKVIVVDPRITESGRIADLWLPIKNGTNMVLVNTFANILINKQFYNKQYVEDHTVGFEEYRSIVENYTPEYAEKVTGIPSEDIVEAMKMYSGAKNAMILYGMGVCQFAQAVDVVKGLASIALLTGNFGRPNVGIGPVRGQNNVQGACDMGALPNVYPGYQSVTDDAIREKFEKAWGVKLPNKVGYHLTQVPELTLKEDKIKAYYIMGEDPVQSDPDSNEMRETLDKMELVIVQDIFMNKTALHADVILPSTSWGEHEGVFSSADRGFQRFRKAVEPKGDVKPDWEIISKIACAMGYNMHYNNTEEIWNELINLCPNFKGATYKRLEELGGIQWPCPSENHPGTSYLYKGNKFNTPTGKANLFAAEWRPPVEQTDKDYPLVLSTVREVGHYSVRTMTGNCRALQQLADEPGYVQVNPMDAKAKGIIDGELMRISSRRGSVVARALITERANKGAVYMTYQWWVGACNELTSNNLDPVSKTPELKYCAVKIEAIKDQKEAEKFIKDQYDLLKKKMNV

SEQ ID NO: 200 - protein, 709 aa, Clostridium autoethanogenum
MDKKVLTVCPYCGAGCNLYLHVKNGKIIKAEPANGRTNEGSLCLKGHFGWDFLNDPKILTSRIKHPMIRKNGELEEVSWDEAISFTASRLSQIKEKYGPDSIMGTGCARGSGNEANYIMQKFMRAVIGTNNVDHCARVCHAPSVAGLAYVLGNGAMSNGIHEIDDTDLLLIFGYNGAASHPIVAKRIVRAKQKGAKVIVVDPRITESGRIADLWLPIKNGTNMVLVNTFANILINKQFYNKQYVEDHTVGFEEYRSIVENYTPEYAEKVTGIPSEDIVEAMKMYSGAKNAMILYGMGVCQFAQAVDVVKGLASIALLTGNFGRPNVGIGPVRGQNNVQGACDMGALPNVYPGYQSVTDDAIRQKFEKAWGVKLPNKVGYHLTQVPELTLKEDKIKAYYIMGEDPVQSDPDSNEMRETLDKMELVIVQDIFMNKTALHADVILPSTSWGEHEGVFSSADRGFQRFRKAVEPKGDVKPDWEIISKIACAMGYNMHYNNTEEIWNELINLCPNFKGATYKRLEELGGIQWPCPSENHPGTSYLYKGNKFNTPTGKANLFAAEWRPPVEQTDKDYPLVLSTVREVGHYSVRTMTGNCRALQQLADEPGYVQVNPMDAKAKGIIDGELMRISSRRGSVVARALITERANKGAVYMTYQWWVGACNELTSNNLDPVSKTPELKYCAVKIEAIKDQKEAEKFIKDQYDLLKKKMNV

SEQ ID NO: 201 - protein, 709 aa, Clostridium coskatii
MDKKVLTVCPYCGAGCNLYLHVKNGKIIKAEPANGRTNEGSLCLKGHFGWDFLNDPKILTSRIKHPMIRKNGELEEVSWDEAISFTASRLSQIKEKYGPDSIMGTGCARGSGNEANYIMQKFMRAVIGTNNVDHCARVXHAPSVAGLAYVLGNGAMSNGIHEIDDTDLLLIFGYNGAASHPIVAKRIVRAKQKGAKVIVVDPRITESGRIADLWLPIKNGTNMVLVNTFANILINKQFYNKQYVEDHTVGFEEYRSIVENYTPEYAEKVTGIPSEDIVEAMKMYSGAKNAMILYGMGVCQFAQAVDVVKGLASIALLTGNFGRPNVGIGPVRGQNNVQGACDMGALPNVYPGYQSVTDDAIREKFEKAWGVKLPNKIGYHLTQVPELTLKEDKIKAYYIMGEDPVQSDPDSNEMRETLDKMELVIVQDIFMNKTALHADVILPSTSWGEHEGVFSSADRGFQRFRKAVEPKGDVKPDWEIISEIACAMGYNMHYNNTEEIWNELINLCPNFKGATYKRLEELGGIQWPCPSENHPGTSYLYKGNKFNTPTGKANLFAAEWRPPVEQTDKDYPLVLSTVREVGHYSVRTMTGNCRALQQLADEPGYVQINPMDAKAEGIIDGELMRISSRRGSVVARALVTERANKGAVYMTYQWWVGACNELTSNNLDPVSKTPELKYCAVKIEAIKDQKEAEKFIKDQYDLLKKKMNV

SEQ ID NO: 202 - protein, 709 aa, Clostridium ragsdalei
MDKKVLTVCPYCGAGCKLYLHVKDGKIIKAEPANGRTNEGSLCLKGRFGWDFLNDPKILTSRIKHPMIRKNGELEEVSWDEAISFTASKLSQIKEKYGPDSIMGTGCARGSGNEANYVMQKFMRAVIGTNNVDHCARVXHAPSVAGLAYVLGNGAMSNGIHEIDDTDLLLIFGYNGAASHPIVAKRIVRAKQKGAKVIVVDPRITESGRIADLWLPIKNGTNMVLVNTFANILINKQFYDKQYVEDHTVGFEEYKSIVEDYTPEYAEKVTGIPAEDIVEAMKMYSSAKNAMILYGMGVCQFAQAVDVVKGLASIALLTGNFGRPNVGIGPVRGQNNVQGACDMGALPNVYPGYQSVTDDAIREKFEKAWGVKLSNKVGYHLTRVPELTLKEDKIKAYYIMGEDPAQSDPDSNEMRETLDKMELVIVQDIFMNKTALHADVILPSTSWGEHEGVFSSADRGFQRFRKAVEPKGDVKPDWEIISEIACAMGYDMHYNNTEEIWDELINLCPNFKGATYKRLDELGGIQWPCPSEDHPGTSYLYKGNKFNTPTGKANLFAAEWRPPIEKTDEEYPLVLSTVREVGHYSVRTMTGNCRALQQLADEPGYVQINPVDAKAKKIIDGELMRVSSRRGSVVARALVTERANKGAVYMTYQWWVGACNELTANNLDPVSKTPELKYCAVKVEAIEDQKEAEKFIKDQYASIKKKMNV

SEQ ID NO: 203 - protein, 711 aa, Paraclostridium bifermentans
MEKKVLTVCPYCGAGCQLYLVVKDNEIVRAEPANGRTNEGNLCLKGYYGWDFLNDPKILTSRLKKPMIRKNGKLEEVEWKEAIDYTASRLNEIREKYGPDAIMGTGSARGPGNEANYVMQKFMRAAIGTNNIDHCARVCHAPSVAGLAYSLGNGAMSNSIPEIENSDLLFIFGYNGADSHPIVARRIIRAKEKGAKLIVTDPRVTESVRISDMWLPIKGGTNMILVNAFANVLIEENLYNKEYVEKYTEGFEEYKEMVKKYTPEYAEKMTNVPAEDIRKAMREYANAKNATILYGMGVCQFGQAVDVVKGLASLALLTGNFGRESVGIGPVRGQNNVQGTCDMGTLPNLFPGYQKVTDDKVRKKFEKAWGVKLSSKPGITLTEVPHLTLKDNKVKAYYIFGEDPVQSDPHASEVRETLDAMEFVVVQDIFMNKTALHADVVLPATSWGEHDGVYSSADRGFQRMRKAVEPKGDVKPDWQIMCEISTAMGYPMSYNNTKEIWDEMISLSPLFAGASYEKIEKQGSVLWPCTDESHKGTPYLYEGNKFETESGKGKLFACEWRPPKEIPDEEYPLVLCTVREVGHYSVRTMTGNCRALRALEDEPGYIQLSIEDASKLGIKDKELVRVSSRRGSILTRALVTDRVIKGATYMTYSWWIGSCNELTIDNLDPISKTPEYKYCAIKVISIKDQDSAEQYIKDEYEKIRKQMLIEK

SEQ ID NO: 204 - protein, 364 aa, synthetic construct
MKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKCLLSKFKKGAWLVNTARGAICVAEDVAAALCSGQLRGYGGDVWSPQPAPKDHPWRDMRNKYGAGNAMTPHYSGTTLDAQTRYAEGTKNILESFFTGKFDYRPQDIILLNGEYITKAYGKHDKK

SEQ ID NO: 205 - protein, 370 aa, synthetic construct
MKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKCLLSKFKKGAWLVNTARGAICVAEDVAAALCSGQLRGYGGDVWSPQPAPKDHPWRDMRNKYGAGNAMTPHYSGTTLDAQTRYAEGTKNILESFFTGKFDYRPQDIILLNGEYITKAYGKHDKKHHHHHH

SEQ ID NO: 206 - protein, 20 aa, synthetic construct
  REPEAT 1..5: note = single repeat of GGGGS
  VARIANT 6..20: note = up to 3 copies of GGGGS can be deleted
GGGGSGGGGSGGGGSGGGGS